(19) United States
(12) Patent Application Publication
(10) Pub. No.: US 2015/0166505 A1
(43) Pub. Date: Jun. 18, 2015

(54) PYRIDINYL-SUBSTITUTED PYRAZOLYL CARBOXAMIDES

(71) Applicant: GRÜNENTHAL GMBH, AACHEN (DE)

(72) Inventors: FELIX VOSS, AACHEN (DE); SONJA NORDHOFF, AACHEN (DE); SEBASTIAN WACHTEN, AACHEN (DE); ANDRÉ WELBERS, KOLN (DE); STEFANIE RITTER, KOLN (DE)

(73) Assignee: GRÜNENTHAL GMBH, AACHEN (DE)

(21) Appl. No.: 14/574,447

(22) Filed: Dec. 18, 2014

(30) Foreign Application Priority Data
Dec. 18, 2013 (EP) 13005894.4
Jul. 4, 2014 (EP) 14002290.6

(51) Int. Cl. C07D 401/04; C07D 405/14; A61K 31/4439; C07D 413/14; C07D 401/14; A61K 31/444

(52) U.S. Cl. CPC C07D 401/04; C07D 401/14; A61K 31/444; A61K 31/4439; C07D 413/14; C07D 405/14

(57) ABSTRACT

The invention relates to pyrazolyl-based carboxamide compounds useful as ICRAC inhibitors, to pharmaceutical compositions containing these compounds and to methods of using these compounds for the treatment and/or prophylaxis of diseases and/or disorders, in particular inflammatory diseases and/or inflammatory disorders.

### PRIORITY

This application claims priority of European Patent Applications Nos. 13005894.4 and 14002290.6, both filed on Dec. 18, 2013, the entire contents of which are hereby incorporated herein by reference.

### FIELD OF THE INVENTION

The invention relates to pyrazol-3-yl-carboxylic acid amides bearing an pyridinyl substituent, useful for inhibition of the Calcium Release Activated Calcium channel (CRAC) and hence for inhibition of the Calcium Release Activated Calcium current (ICRAC), to pharmaceutical compositions containing these compounds and also to these compounds for the use in immuosupression and in the treatment and/or prophylaxis of conditions, diseases and/or disorders, in particular immune disorders, inflammatory conditions and allergic diseases.

### BACKGROUND OF THE INVENTION

Calcium-conducting channels in the plasma membrane can appear very diverse (Parekh & Putney 2005) including voltage-gated ion channels (VOC's), receptor-operated ion channels (ROC's), but also store-operated channels (SOC's; Putney, 1986) that are activated in response to a decrease of the intraluminal Calcium concentration within i.e. the endoplasmic reticulum (ER). The latter have been demonstrated to serve as the main Calcium entry mechanisms in non-excitable cells.

Amongst the distinct SOCs, the CRAC current (ICRAC) is certainly characterized best and displays biophysical features such as high selectivity for Calcium ions, low conductance, and inward rectification (Hoth & Penner, 1992; Hoth & Penner, 1993; Parekh & Penner, 1997; Lepple-Wienhues & Cahalan, 1996; Kerschbaum & Cahalan, 1999). There's substantial evidence that the channels conducting CRAC predominantly rely on two proteins, Orai1 and Stim1 (Roos et al., 2005; Feske et al., 2006; Peinelt et al., 2006). Orai1 constitutes the channel pore within the plasma membrane (Prakriya et al., 2006; Vig et al., 2006), whereas Stim1 has been demonstrated to function as the sensor of the luminal Calcium concentration (Liou et al., 2005; Zhang et al., 2006).

In a physiological setting, ICRAC is activated in response to the engagement of cell-surface receptors that positively couple to phospholipase C (PLC). PLC increases the concentration of the soluble messenger inositol-1,4,5-trisphosphate (IP3), which opens ER membrane-resident IP3-receptors. Thus, IP3 triggers the release of Calcium from internal stores resulting in a drop of the luminal Calcium concentration (Lewis, 1999), which is sensed by Stim1. The Stim1 molecule undergoes conformational changes inducing clustering with other Stim1 molecules just underneath the plasma membrane. At these sites, Stim1 can open the Orai1 pore by bridging the ER-PM gap with its C-terminal tail (Zhang et al., 2005; Luik et al., 2006; Soboloff et al. 2006, Wu et al. 2006; Li et al., 2007).

The above described process serves in signaling pathways of immune cells such as lymphocytes and mast cells. I.e. the activation of antigen or Fc receptors stimulates the release of Calcium from intracellular stores, and subsequent activation of ICRAC that impacts on downstream processes such as gene expression and cytokine release (Feske, 2007; Gwack et al., 2007; Oh-hora & Rao 2008).

The major contribution ICRAC provides to these signaling events has been convincingly demonstrated in patients suffering from severe combined immunodeficiency (SCID) due to a defect in T-cell activation. T cells and fibroblasts from these patients exhibited a strong attenuation of store-operated Calcium entry carried by ICRAC (Feske et al., 2006). This suggests CRAC channel modulators to serve as treatment in disease states caused by activated inflammatory cells.

The activation of antigen or Fc receptors stimulates the release of Calcium from intracellular stores and subsequent, sustained activation of ICRAC. Calcium carried by ICRAC activates calcineurin (CaN), which dephosphorylates the transcription factor NFAT. Upon dephosphorylation, NFAT shuttles into the nucleus and regulates gene expression in various ways depending on the nature of the stimulus as well as on the cell/tissue type.

NFAT participates in the transactivation of cytokine genes that regulate T-cell proliferation and other genes that control immune responses. Taking into account that the expression of cytokines such as IL-2, IL-4, IL-5, IL-8, IL-13, tumor necrosis factor alpha (TNFα), granulocyte colony-stimulating factor (G-CSF), and gamma-interferon (INFγ) is prone to be controlled via transcriptional elements for NFAT, the impact of the ICRAC/CaN/NFAT signaling pathway on pro-inflammatory processes becomes apparent. The inhibition of this pathway has been demonstrated to be efficacious in patients by the use of drugs such as CsA and FK506, which act by inhibiting CaN.

A hallmark of ICRAC signaling in immune cells is that downstream processes such as gene expression rely on sustained Calcium entry rather than transient signals. However, Calcium entry is essential for other processes that can be independent of CaN/NFAT. Direct, Calcium-mediated release of substances (degranulation) such as histamine, heparin, and TNFα occur in i.e. mast cells, and are of rather acute nature. On the molecular level, this already points towards a differentiation potential for ICRAC blockers from calcineurin inhibitors.

Recent findings suggest that CRAC channel modulators can serve as treatment in disease states caused by the activation of inflammatory cells without side effects observed under treatments with i.e. steroids. Such diseases may include but are not limited to asthma, chronic obstructive pulmonary disease, rheumatoid arthritis, inflammatory bowel disease, glomerulonephritis, neuroinflammatory diseases such as multiple sclerosis, and disorders of the immune system.

U.S. Pat. No. 6,958,339, WO 2009/076454 A1, WO 2009/089305 A1, and WO 2010/122089 A1 each disclose a series of pyrazole carboxylic acid amide derivatives that are said to possess CRAC channel inhibitory activity which are believed to be useful in the treatment of allergic, inflammatory or autoimmune diseases. Other small molecules possessing structurally different scaffolds as ICRAC inhibitors are known for instance from WO2005/009539, WO 2007/087427 A2 and WO 2007/087441 A2. Pyrazole carboxylic acid amides as biologically active compounds are also known in the art, for instance from EP 1176140 B1, US 2006/0100208 A1, WO 2005/016877 A2, WO 2006/076202 A1, WO 2007/002559 A1, WO 2007/024744 A2, WO 2009/011850 A2 and WO 2009/027393 A2.

Pyridinyl substituted aryl and heteroaryl carboxamide derivatives as CRAC channel inhibitors have been disclosed in WO 2013/164769 and US 2006/0173006.

### SUMMARY OF THE INVENTION

The present invention describes a new class of small molecule that is useful for the inhibition of the calcium release activated calcium channel current (thereafter ICRAC inhibitors).

It was therefore an object of the invention to provide novel compounds, preferably having advantages over the prior-art compounds. The compounds should be suitable in particular as pharmacological active ingredients in pharmaceutical compositions, preferably in pharmaceutical compositions for the treatment and/or prophylaxis of disorders or diseases which are at least partially mediated by CRAC channels.

This object is achieved by the subject matter described herein.

It has surprisingly been found that the compounds of general formula (I), as given below, display potent inhibitory activity against to CRAC channels and are therefore particularly suitable for the prophylaxis and/or treatment of disorders or diseases which are at least partially mediated by CRAC channels.

A first aspect of the present invention therefore relates to a compound of general formula (I),

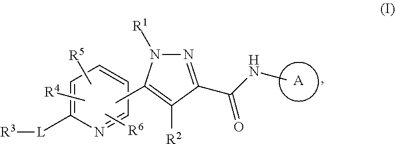

wherein
R¹ denotes H, C₁₋₄-alkyl or C₃₋₆-cycloalkyl;
R² denotes H; F; Cl; Br; CN; CF₃; CF₂H; CFH₂; C₁₋₄-alkyl; OH; O—C₁₋₄-alkyl; NH₂; N(H)C₁₋₄-alkyl; N(C₁₋₄-alkyl)₂;
L represents bond, O, C₁₋₄-alkylene, C₁₋₄-alkylene-O or O—C₁₋₄-alkylene;
R³ is selected from the group consisting of Cl, OH, CN; CF₃; CF₂H; CFH₂; C₁₋₄-alkyl; C₃₋₆-cycloalkyl; 3 to 7 membered heterocycloalkyl; 5- to 6-membered heteroaryl; C(═O)OH; C(═O)O—C₁₋₄-alkyl; C(═O)NH₂; C(═O)N(H)C₁₋₄-alkyl; C(═O)N(C₁₋₄-alkyl)₂; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl); N(C₁₋₄-alkyl)S(═O)₂(C₁₋₄-alkyl); S(═O)₂C₁₋₄-alkyl; S(═O)C₁₋₄-alkyl; S(═O)₂NH₂; S(═O)₂N(H)C₁₋₄-alkyl; S(═O)₂N(C₁₋₄-alkyl)₂;
R⁴, R⁵ and R⁶ are each independently selected from the group consisting H; F; Cl; Br; CN; CF₃; CF₂H; CFH₂; C₁₋₄-alkyl; C₃₋₆-cycloalkyl; 3 to 7 membered heterocycloalkyl; OH; O—C₁₋₄-alkyl; OCF₃; OCF₂H; OCFH₂; O C₃₋₆-cycloalkyl; O-(3 to 7 membered heterocycloalkyl); NH₂; N(H)C₁₋₄-alkyl; N(C₁₋₄-alkyl)₂; NH(C═O)(C₁₋₄-alkyl);
A represents phenyl or 5- to 6-membered heteroaryl,
   wherein said heteroaryl, cycloalkyl and heterocycloalkyl each independently are unsubstituted or mono- or polysubstituted;
   and wherein said C₁₋₄-alkyl and C₁₋₄-alkylene each independently are linear or branched, and each independently are unsubstituted or mono- or polysubstituted;
      optionally in the form of a single stereoisomer or a mixture of stereoisomers, in the form of the free compound and/or a physiologically acceptable salt thereof and/or a physiologically acceptable solvate thereof.

### DETAILED DESCRIPTION

The term “single stereoisomer” preferably means in the sense of the present invention an individual enantiomer or diastereomer. The term “mixture of stereoisomers” means in the sense of this invention mixtures of enantiomers and/or diastereomers in any mixing ratio including racemates.

The term “physiologically acceptable salt” preferably comprises in the sense of this invention a salt of at least one compound according to the present invention and at least one physiologically acceptable acid or base.

A physiologically acceptable salt of at least one compound according to the present invention and at least one physiologically acceptable acid preferably refers in the sense of this invention to a salt of at least one compound according to the present invention with at least one inorganic or organic acid which is physiologically acceptable—in particular when used in human beings and/or other mammals.

A physiologically acceptable salt of at least one compound according to the present invention and at least one physiologically acceptable base preferably refers in the sense of this invention to a salt of at least one compound according to the present invention as an anion with at least one preferably inorganic cation, which is physiologically acceptable—in particular when used in human beings and/or other mammals.

The term “physiologically acceptable solvate” preferably comprises in the sense of this invention an adduct of one compound according to the present invention and/or a physiologically acceptable salt of at least one compound according to the present invention with distinct molecular equivalents of one solvent or more solvents.

The term “C₁₋₄-alkyl” comprises in the sense of this invention acyclic saturated, aliphatic hydrocarbon residues, which can be branched or unbranched and also unsubstituted or mono- or polysubstituted, which contain 1 to 4 carbon atoms respectively. Preferred C₁₋₄-alkyl residues are selected from the group consisting of methyl, ethyl, n-propyl, 2-propyl, n-butyl, isobutyl, sec.-butyl and tert.-butyl.

The term “C₁₋₄-alkylene” comprises in the sense of this invention bivalent acyclic saturated, aliphatic hydrocarbon residues, which can be branched or unbranched and also unsubstituted or mono- or polysubstituted, which contain 1 to 4 carbon atoms respectively. Preferred C₁₋₄-alkylene residues are selected from the group consisting of methylene, 1,2-ethylene, 1,3-propylene, 1,4-butylene, 1,1-ethylene, 1,1-propylene, 1,2-propylene, 1,1-butylene, 1,2-butylene, 1,3-butylene and 2,3-butylene.

The term “C₃₋₆-cycloalkyl” means for the purposes of this invention cyclic aliphatic hydrocarbons containing 3, 4, 5 or 6 carbon atoms, wherein the hydrocarbons in each case can be unsubstituted or mono- or polysubstituted. The C₃₋₆-cycloalkyl can be bound to the respective superordinate general structure via any desired and possible ring member of the C₃₋₆-cycloalkyl. Preferred C₃₋₆-cycloalkyls are selected from the group consisting of cyclopropyl, cyclobutyl, cyclopentyl, cyclohexyl, in particular cyclopropyl.

The term “5- to 6-membered heteroaryl” for the purpose of this invention represents a 5 or 6-membered cyclic aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted; in the case of substitution on the heteroaryl, the substituents can be the same or different and be in any desired and possible position of the heteroaryl. The binding to the superordinate general structure can be carried out via any desired and possible ring member of the heteroaryl residue if not indicated otherwise. It is preferable for the heteroaryl residue to be selected from the group consisting of benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl.

In relation to the terms “C₁₋₄-alkyl, C₁₋₄-alkylene, C₃₋₆-cycloalkyl and 3 to 7 membered heterocycloalkyl”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; CN; CF₃; CF₂H; CFH₂; CF₂Cl; CFCl₂; C₁₋₄-alkyl; C₃₋₆-cycloalkyl; 3 to 7 membered heterocycloalkyl; aryl; heteroaryl; aryl, heteroaryl, C₃₋₆-cycloalkyl or 3 to 7 membered heterocycloalkyl, each connected via a C₁₋₄-alkyl; C(═O)—(C₁₋₄-alkyl); C(═O)—(C₃₋₆-cycloalkyl); C(═O)-(3 to 7 membered heterocycloalkyl); C(═O)-(aryl); C(═O)-(heteroaryl); C(═O)OH; C(═O)—O(C₁₋₄-alkyl); C(═O)—O(C₃₋₆-cycloalkyl); C(═O)—O(3 to 7 membered heterocycloalkyl); C(═O)—O(aryl); C(═O)—O(heteroaryl); C(═O)—NH₂; C(═O)—N(H)(C₁₋₄-alkyl); C(═O)—N(H)(C₃₋₆-cycloalkyl); C(═O)—N(H)(3 to 7 membered heterocycloalkyl); C(═O)—N(H)(aryl); C(═O)—N(H)(heteroaryl); C(═O)—N(C₁₋₄-alkyl)₂; C(═O)—N(C₁₋₄-alkyl)(C₃₋₆-cycloalkyl); C(═O)—N(C₁₋₄-alkyl)(3 to 7 membered heterocycloalkyl); C(═O)—N(C₁₋₄-alkyl)(aryl); C(═O)—N(C₁₋₄-alkyl)(heteroaryl); OH; ═O; O—(C₁₋₄-alkyl); O—(C₃₋₆-cycloalkyl); O-(3 to 7 membered heterocyclic residue); O-(aryl); O-(heteroaryl); OCF₃; OCF₂H; OCFH₂; OCF₂Cl; OCFCl₂; O—C(═O)—(C₁₋₄-alkyl); O—C(═O)—(C₃₋₆-cycloalkyl); O—C(═O)-(3 to 7 membered heterocycloalkyl); O—C(═O)-(aryl); C(═O)-(heteroaryl); O—C(═O)—NH₂; O—C(═O)—N(H)(C₁₋₄-alkyl); O—C(═O)—N(H)(C₃₋₆-cycloalkyl); O—C(═O)—N(H)(3 to 7 membered heterocycloalkyl); O—C(═O)—N(H)(aryl); O—C(═O)—N(H)(heteroaryl); O—C(═O)—N(C₁₋₄-alkyl)₂; O—C(═O)—N(C₁₋₄-alkyl)(C₃₋₆-cycloalkyl); O—C(═O)—N(C₁₋₄-alkyl)(3 to 7 membered heterocycloalkyl); O—C(═O)—N(C₁₋₄-alkyl)(aryl); O—C(═O)—N(C₁₋₄-alkyl)(heteroaryl); NH₂; N(H)(C₁₋₄-alkyl); N(H)(C₃₋₆-cycloalkyl); N(H)(3 to 7 membered heterocycloalkyl); N(H)(aryl); N(H)(heteroaryl); N(C₁₋₄-alkyl)₂; N(C₁₋₄-alkyl)(C₃₋₆-cycloalkyl); N(C₁₋₄-alkyl)(3 to 7 membered heterocycloalkyl); N(C₁₋₄-alkyl)(aryl); N(C₁₋₄-alkyl)(heteroaryl); N(H)—C(═O)—(C₁₋₄-alkyl); N(H)—C(═O)—(C₃₋₆-cycloalkyl); N(H)—C(═O)-(3 to 7 membered heterocycloalkyl); N(H)—C(═O)-(aryl); N(H)—C(═O)-(heteroaryl); N(C₁₋₄-alkyl)-C(═O)—(C₁₋₄-alkyl); N(C₁₋₄-alkyl)-C(═O)—(C₃₋₆-cycloalkyl); N(C₁₋₄-alkyl)-C(═O)-(3 to 7 membered heterocycloalkyl); N(C₁₋₄-alkyl)-C(═O)-(aryl); N(C₁₋₄-alkyl)-C(═O)-(heteroaryl); N(H)—S(═O)₂—(C₁₋₄-alkyl); N(H)—S(═O)₂—(C₃₋₆-cycloalkyl); N(H)—S(═O)₂-(3 to 7 membered heterocycloalkyl); N(H)—S(═O)₂-(aryl); N(H)—S(═O)₂-(heteroaryl); N(C₁₋₄-alkyl)-S(═O)₂—(C₁₋₄-alkyl); N(C₁₋₄-alkyl)-S(═O)₂—(C₃₋₆-cycloalkyl); N(C₁₋₄-alkyl)-S(═O)₂-(3 to 7 membered heterocycloalkyl); N(C₁₋₄-alkyl)-S(═O)₂-(aryl); N(C₁₋₄-alkyl)-S(═O)₂-(heteroaryl); N(H)—C(═O)—O(C₁₋₄-alkyl); N(H)—C(═O)—O(C₃₋₆-cycloalkyl); N(H)—C(═O)—O (3 to 7 membered heterocycloalkyl); N(H)—C(═O)—O(aryl); N(H)—C(═O)—O(heteroaryl); N(C₁₋₄-alkyl)-C(═O)—O(C₁₋₄-alkyl); N(C₁₋₄-alkyl)-C(═O)—O(C₃₋₆-cycloalkyl); N(C₁₋₄-alkyl)-C(═O)—O (3 to 7 membered heterocycloalkyl); N(C₁₋₄-alkyl)-C(═O)—O(aryl); N(C₁₋₄-alkyl)-C(═O)—O(heteroaryl); N(H)—C(═O)—NH₂; N(H)—C(═O)—N(H)(C₁₋₄-alkyl); N(H)—C(═O)—N(E)(C₃₋₆-cycloalkyl); N(H)—C(═O)—N(H)(3 to 7 membered heterocycloalkyl); N(H)—C(═O)—N(H)(aryl); N(H)—C(═O)—N(H)(heteroaryl); N(C₁₋₄-alkyl)-C(═O)—NH₂; N(C₁₋₄-alkyl)-C(═O)—N(H)(C₁₋₄-alkyl); N(C₁₋₄-alkyl)-C(═O)—N(H)(C₃₋₆-cycloalkyl); N(C₁₋₄-alkyl)-C(═O)—N(H)(3 to 7 membered heterocycloalkyl); N(C₁₋₄-alkyl)-C(═O)—N(H)(aryl); N(C₁₋₄-alkyl)-C(═O)—N(H)(heteroaryl); N(H)—C(═O)—N(C₁₋₄-alkyl)₂; N(H)—C(═O)—N(C₁₋₄-alkyl)(C₃₋₆-cycloalkyl); N(H)—C(═O)—N(C₁₋₄-alkyl)(3 to 7 membered heterocycloalkyl); N(H)—C(═O)—N(C₁₋₄-alkyl)(aryl); N(H)—C(═O)—N(C₁₋₄-alkyl)(heteroaryl); N(C₁₋₄-alkyl)-C(═O)—N(C₁₋₄-alkyl)₂; N(C₁₋₄-alkyl)-C(═O)—N(C₁₋₄-alkyl)(C₃₋₆-cycloalkyl); N(C₁₋₄-alkyl)-C(═O)—N(C₁₋₄-alkyl)(3 to 7 membered heterocycloalkyl); N(C₁₋₄-alkyl)-C(═O)—N(C₁₋₄-alkyl)(aryl); N(C₁₋₄-alkyl)-C(═O)—N(C₁₋₄-alkyl)(heteroaryl); S—(C₃₋₆-cycloalkyl); S-(3 to 7 membered heterocycloalkyl); S-(aryl); S-(heteroaryl); SCF₃; S(═O)₂OH; S(═O)—(C₁₋₄-alkyl); S(═O)—(C₃₋₆-cycloalkyl); S(═O)-(3 to 7 membered heterocycloalkyl); S(═O)-(aryl); S(═O)-(heteroaryl); S(═O)₂—(C₁₋₄-alkyl); S(═O)₂—(C₃₋₆-cycloalkyl); S(═O)₂-(3 to 7 membered heterocycloalkyl); S(═O)₂-(aryl); S(═O)₂-(heteroaryl); S(═O)₂—O(C₁₋₄-alkyl); S(═O)₂—O(C₃₋₆-cycloalkyl); S(═O)₂—O(3 to 7 membered heterocycloalkyl); S(═O)₂—O(aryl); S(═O)₂—O(heteroaryl); S(═O)₂—N(H)(C₁₋₄-alkyl); S(═O)₂—N(H)(C₃₋₆-cycloalkyl); S(═O)₂—N(H)(3 to 7 membered heterocycloalkyl); S(═O)₂—N(H)(aryl); S(═O)₂—N(H)(heteroaryl); S(═O)₂—N(C₁₋₄-alkyl)₂; S(═O)₂—N(C₁₋₄-alkyl)(C₃₋₆-cycloalkyl); S(═O)₂—N(C₁₋₄-alkyl)(3 to 7 membered heterocycloalkyl); S(═O)₂—N(C₁₋₄-alkyl)(aryl); S(═O)₂—N(C₁₋₄-alkyl)(heteroaryl).

The term “polysubstituted” with respect to polysubstituted residues and groups includes the polysubstitution of these residues and groups either on different or on the same atoms, for example trisubstituted on the same carbon atom, as in the case of CF₃, CH₂CF₃ or 1,1-difluorocyclohexyl, or at various points, as in the case of CH(OH)—CH═CH—CHCl₂ or 1-chloro-3-fluorocyclohexyl. A substituent can if appropriate for its part in turn be mono- or polysubstituted. The multiple substitution can be carried out using the same or using different substituents.

Preferred substituents of “C₁₋₄-alkyl, C₁₋₄-alkylene, C₃₋₆-cycloalkyl and 3 to 7 membered heterocycloalkyl” are selected from the group consisting of F; Cl; CN; ═O; CF₃; CF₂H; CFH₂; CF₂Cl; CFCl₂; C₁₋₄-alkyl C(═O)—H; C(═O)—C₁₋₄-alkyl; C(═O)—OH; C(═O)—O—C₁₋₄-alkyl; C(═O)—N(H)(OH); C(═O)—NH₂; C(═O)—N(H)(C₁₋₄-alkyl); C(═O)—N(C₁₋₄-alkyl)₂; OH; OCF₃; OCF₂H; OCFH₂; OCF₂Cl; OCFCl₂; O—C₁₋₄-alkyl; O—C(═O)—C₁₋₄-alkyl; O—C(═O)—O—C₁₋₄-alkyl; O—(C═O)—N(H)(C₁₋₄-alkyl); O—C(═O)—N(C₁₋₄-alkyl)₂; O—S(═O)₂—C₁₋₄-alkyl; O— S(═O)₂—OH; O—S(═O)₂—O—C₁₋₄-alkyl; O—S(═O)₂—NH₂; O—S(═O)₂—N(H)(C₁₋₄-alkyl); O—S(═O)₂—N(C₁₋₄-alkyl)₂; NH₂; N(H)(C₁₋₄-alkyl); N(C₁₋₄-alkyl)₂; N(H)—C(═O)—C₁₋₄-alkyl; N(H)—C(═O)—O—C₁₋₄-alkyl; N(H)—C(═O)—NH₂; N(H)—C(═O)—N(H)(C₁₋₄-alkyl); N(H)—C(═O)—N(C₁₋₄-alkyl)₂; N(C₁₋₄-alkyl)-C(═O)—C₁₋₄-alkyl; N(C₁₋₄-alkyl)-C(═O)—O—C₁₋₄-alkyl; N(C₁₋₄-alkyl)-C(═O)—NH₂; N(C₁₋₄-alkyl)-C(═O)—N(H)(C₁₋₄-alkyl); N(C₁₋₄-alkyl)-C(═O)—N(C₁₋₄-alkyl)₂; N(H)—S(═O)₂—OH; N(H)—S(═O)₂—C₁₋₄-alkyl; N(H)—S(═O)₂—O—C₁₋₄-alkyl; N(H)—S(═O)₂—NH₂; N(H)—S(═O)₂—N(H)(C₁₋₄-alkyl); N(H)—S(═O)₂—N(C₁₋₄-alkyl)₂; N(C₁₋₄-alkyl)-S(═O)₂—OH; N(C₁₋₄-alkyl)-S(═O)₂—C₁₋₄-alkyl; N(C₁₋₄-alkyl)-S(═O)₂—O—C₁₋₄-alkyl; N(C₁₋₄-alkyl)-S(═O)₂—NH₂; N(C₁₋₄-alkyl)-S(═O)₂—N(H)(C₁₋₄-alkyl); N(C₁₋₄-alkyl)-S(═O)₂—N(C₁₋₄-alkyl)₂; SH; SCF₃; SCF₂H; SCFH₂; SCF₂Cl; SCFCl₂; S—C₁₋₄-alkyl; S(═O)—C₁₋₄-alkyl; S(═O)₂—C₁₋₄-alkyl; S(═O)₂—OH; S(═O)₂—O—C₁₋₄-alkyl; S(═O)₂—NH₂; S(═O)₂—N(H)(C₁₋₄-alkyl); and S(═O)₂—N(C₁₋₄-alkyl)₂.

Particularly preferred substituents of “C₁₋₄-alkyl, C₁₋₄-alkylene, C₃₋₆-cycloalkyl and 3 to 7 membered heterocycloalkyl” are selected from the group consisting of F; Cl; CF₃; CN; ═O; C₁₋₄-alkyl; C(═O)—H; C(═O)—C₁₋₄-alkyl; C(═O)—OH; C(═O)—O—C₁₋₄-alkyl; C(═O)—NH₂; C(═O)—N(H)(C₁₋₄-alkyl); C(═O)—N(C₁₋₄-alkyl)₂; OH; O—C₁₋₄-alkyl; O—C(═O)—C₁₋₄-alkyl; OCF₃; NH₂; N(H)(C₁₋₄-alkyl); N(C₁₋₄-alkyl)₂; N(H)—C(═O)—C₁₋₄-alkyl; N(H)—S(═O)₂—C₁₋₄-alkyl; N(C₁₋₄-alkyl)-S(═O)₂—C₁₋₄-alkyl; N(H)—C(═O)—NH₂; N(H)—C(═O)—N(H)(C₁₋₄-alkyl); N(H)—C(═O)—N(C₁₋₄-alkyl)₂; N(H)—S(═O)₂—NH₂; N(H)—S(═O)₂—N(H)(C₁₋₄-alkyl); N(H)—S(═O)₂—N(C₁₋₄-alkyl)₂; N(C₁₋₄-alkyl)-S(═O)₂—NH₂; N(C₁₋₄-alkyl)-S(═O)₂—N(H)(C₁₋₄-alkyl); N(C₁₋₄-alkyl)-S(═O)₂—N(C₁₋₄-alkyl)₂; SH; SCF₃; S—C₁₋₄-alkyl; S(═O)₂C₁₋₄-alkyl; S(═O)₂OH; S(═O)₂O—C₁₋₄-alkyl and S(═O)₂—NH₂; S(═O)₂—N(H)(C₁₋₄-alkyl); and S(═O)₂—N(C₁₋₄-alkyl)₂.

More preferred substituents of “C₁₋₄-alkyl, C₁₋₄-alkylene, C₃₋₆-cycloalkyl and 3 to 7 membered heterocycloalkyl” are selected from the group consisting of F; Cl; CF₃; CN; ═O; C₁₋₄-alkyl; C(═O)—C₁₋₄-alkyl; C(═O)—OH; C(═O)—O—C₁₋₄-alkyl; C(═O)—NH₂; C(═O)—N(H)(C₁₋₄-alkyl); C(═O)—N(C₁₋₄-alkyl)₂; OH; O—C₁₋₄-alkyl; O—C(═O)—C₁₋₄-alkyl; OCF₃; NH₂; N(H)(C₁₋₄-alkyl); N(C₁₋₄-alkyl)₂; N(H)—C(═O)—C₁₋₄-alkyl; N(H)—S(═O)₂—C₁₋₄-alkyl; N(C₁₋₄-alkyl)-S(═O)₂—C₁₋₄-alkyl; N(H)—C(═O)—NH₂; N(H)—C(═O)—N(H)(C₁₋₄-alkyl); N(H)—C(═O)—N(C₁₋₄-alkyl)₂; N(C₁₋₄-alkyl)-S(═O)₂—NH₂; N(C₁₋₄-alkyl)-S(═O)₂—N(H)(C₁₋₄-alkyl); N(C₁₋₄-alkyl)-S(═O)₂—N(C₁₋₄-alkyl)₂; S(═O)₂C₁₋₄-alkyl; S(═O)₂OH; S(═O)₂O—C₁₋₄-alkyl; S(═O)₂—NH₂; S(═O)₂—N(H)(C₁₋₄-alkyl) and S(═O)₂—N(C₁₋₄-alkyl)₂.

Most preferred substituents of “C₁₋₄-alkyl” and of “C₁₋₄-alkylene” are selected from the group consisting of F; Cl; CF₃; C(═O)—OH; C(═O)—NH₂; C(═O)—N(H)(C₁₋₄-alkyl); C(═O)— N(C₁₋₄-alkyl)₂; OH; O—C₁₋₄-alkyl; NH₂; N(H)(C₁₋₄-alkyl); N(C₁₋₄-alkyl)₂; N(H)—C(═O)—C₁₋₄-alkyl; N(H)—S(═O)₂—C₁₋₄-alkyl; N(C₁₋₄-alkyl)S(═O)₂—C₁₋₄-alkyl; N(H)—S(═O)₂—NH₂; S(═O)₂—C₁₋₄-alkyl, S(═O)₂—NH₂, S(═O)₂—N(C₁₋₄-alkyl)₂ and S(═O)₂—N(H)(C₁₋₄-alkyl).

Particularly preferred substituents of “C₃₋₆-cycloalkyl and 3 to 7 membered heterocycloalkyl” are selected from the group consisting of F; Cl; CF₃; CN; ═O; C₁₋₄-alkyl; CO₂H; C(═O)O—C₁₋₄-alkyl; CONH₂; C(═O)N(H)C₁₋₄-alkyl; C(═O)N(C₁₋₄-alkyl)₂; OH; O—C₁₋₄-alkyl; OCF₃; O—C(═O)—C₁₋₄-alkyl; NH₂; NH—C₁₋₄-alkyl; N(C₁₋₄-alkyl)₂; NH—C(═O)—C₁₋₄-alkyl; N(C₁₋₄-alkyl)-C(═O)—C₁₋₄-alkyl; S(═O)₂—C₁₋₄-alkyl; S(═O)₂—NH₂, S(═O)₂—N(C₁₋₄-alkyl)₂ and S(═O)₂—N(H)—C₁₋₄-alkyl.

In relation to the terms “aryl” and “heteroaryl”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; Br; NO₂; CN; CF₃; CF₂H; CFH₂; CF₂Cl; CFCl₂; C₁₋₄-alkyl; C₃₋₆-cycloalkyl; 3 to 7 membered heterocycloalkyl; aryl; heteroaryl; aryl, heteroaryl, C₃₋₆-cycloalkyl or 3 to 7 membered heterocycloalkyl, each connected via a C₁₋₄-alkyl; C(═O)H; C(═O)—(C₁₋₄-alkyl); C(═O)—(C₃₋₆-cycloalkyl); C(═O)-(3 to 7 membered heterocycloalkyl); C(═O)-(aryl); C(═O)-(heteroaryl); C(═O)OH; C(═O)—O(C₁₋₄-alkyl); C(═O)—O(C₃₋₆-cycloalkyl); C(═O)—O(3 to 7 membered heterocycloalkyl); C(═O)—O(aryl); C(═O)—O(heteroaryl); C(═O)—NH₂; C(═O)—N(H)(C₁₋₄-alkyl); C(═O)—N(H)(C₃₋₆-cycloalkyl); C(═O)—N(H)(3 to 7 membered heterocycloalkyl); C(═O)—N(H)(aryl); C(═O)—N(H)(heteroaryl); C(═O)—N(C₁₋₄-alkyl)₂; C(═O)—N(C₁₋₄-alkyl)(C₃₋₆-cycloalkyl); C(═O)—N(C₁₋₄-alkyl)(3 to 7 membered heterocycloalkyl); C(═O)—N(C₁₋₄-alkyl)(aryl); C(═O)—N(C₁₋₄-alkyl)(heteroaryl); OH; ═O; O—(C₁₋₄-alkyl); O—(C₃₋₆-cycloalkyl); O-(3 to 7 membered heterocycloalkyl); O-(aryl); O-(heteroaryl); OCF₃; OCF₂H; OCFH₂; OCF₂Cl; OCFCl₂; O—C(═O)—(C₁₋₄-alkyl); O—C(═O)—(C₃₋₆-cycloalkyl); O—C(═O)-(3 to 7 membered heterocycloalkyl); O—C(═O)-(aryl); C(═O)-(heteroaryl); O—C(═O)—NH₂; O—C(═O)—N(H)(C₁₋₄-alkyl); O—C(═O)—N(H)(C₃₋₆-cycloalkyl); O—C(═O)—N(H)(3 to 7 membered heterocycloalkyl); O—C(═O)—N(H)(aryl); O—C(═O)—N(H)(heteroaryl); O—C(═O)—N(C₁₋₄-alkyl)₂; O—C(═O)—N(C₁₋₄-alkyl)(C₃₋₆-cycloalkyl); O—C(═O)—N(C₁₋₄-alkyl)(3 to 7 membered heterocycloalkyl); O—C(═O)—N(C₁₋₄-alkyl)(aryl); O—C(═O)—N(C₁₋₄-alkyl)(heteroaryl); NH₂; N(H)(C₁₋₄-alkyl); N(H)(C₃₋₆-cycloalkyl); N(H)(3 to 7 membered heterocycloalkyl); N(H)(aryl); N(H)(heteroaryl); N(C₁₋₄-alkyl)₂; N(C₁₋₄-alkyl)(C₃₋₆-cycloalkyl); N(C₁₋₄-alkyl)(3 to 7 membered heterocycloalkyl); N(C₁₋₄-alkyl) (aryl); N(C₁₋₄-alkyl)(heteroaryl); N(H)—C(═O)—(C₁₋₄-alkyl); N(H)—C(═O)—(C₃₋₆-cycloalkyl); N(H)—C(═O)-(3 to 7 membered heterocycloalkyl); N(H)—C(═O)-(aryl); N(H)—C(═O)-(heteroaryl); N(C₁₋₄-alkyl)-C(═O)—(C₁₋₄-alkyl); N(C₁₋₄-alkyl)-C(═O)—(C₃₋₆-cycloalkyl); N(C₁₋₄-alkyl)-C(═O)-(3 to 7 membered heterocycloalkyl); N(C₁₋₄-alkyl)-C(═O)-(aryl); N(C₁₋₄-alkyl)-C(═O)-(heteroaryl); N(H)—S(═O)₂—(C₁₋₄-alkyl); N(H)—S(═O)₂—(C₃₋₆-cycloalkyl); N(H)—S(═O)₂-(3 to 7 membered heterocycloalkyl); N(H)—S(═O)₂-(aryl); N(H)—S(═O)₂-(heteroaryl); N(C₁₋₄-alkyl)-S(═O)₂—(C₁₋₄-alkyl); N(C₁₋₄-alkyl)-S(═O)₂—(C₃₋₆-cycloalkyl); N(C₁₋₄-alkyl)-S(═O)₂-(3 to 7 membered heterocycloalkyl); N(C₁₋₄-alkyl)-S(═O)₂-(aryl); N(C₁₋₄-alkyl)-S(═O)₂-(heteroaryl); N(H)—C(═O)—O(C₁₋₄-alkyl); N(H)—C(═O)—O(C₃₋₆-cycloalkyl); N(H)—C(═O)—O(3 to 7 membered heterocycloalkyl); N(H)—C(═O)—O(aryl); N(H)—C(═O)—O(heteroaryl); N(C₁₋₄-alkyl)-C(═O)—O(C₁₋₄-alkyl); N(C₁₋₄-alkyl)-C(═O)—O(C₃₋₆-cycloalkyl); N(C₁₋₄-alkyl)-C(═O)—O (3 to 7 membered heterocycloalkyl); N(C₁₋₄-alkyl)-C(═O)—O(aryl); N(C₁₋₄-alkyl)-C(═O)—O(heteroaryl); N(H)—C(═O)—NH₂; N(H)—C(═O)—N(H)(C₁₋₄-alkyl); N(H)—C(═O)—N(H)(C₃₋₆-cycloalkyl); N(H)—C(═O)—N(H)(3 to 7 membered heterocycloalkyl); N(H)—C(═O)—N(H)(aryl); N(H)—C(═O)—N(H)(heteroaryl); N(C₁₋₄-alkyl)-C(═O)—NH₂; N(C₁₋₄-alkyl)-C(═O)—N(H)(C₁₋₄-alkyl); N(C₁₋₄-alkyl)-C(═O)—N(H)(C₃₋₆-cycloalkyl); N(C₁₋₄-alkyl)-C(═O)—N(H)(3 to 7 membered heterocycloalkyl); N(C₁₋₄-alkyl)-C(═O)—N(H)(aryl); N(C₁₋₄-alkyl)-C(═O)—N(H)(heteroaryl); N(H)—C(═O)—N(C₁₋₄-alkyl)₂; N(H)—C(═O)—N(C₁₋₄-alkyl)(C₃₋₆-cycloalkyl); N(H)—C(═O)—N(C₁₋₄-alkyl)(3 to 7 membered heterocycloalkyl); N(H)—C(═O)—N(C₁₋₄-alkyl)(aryl); N(H)—C(═O)—N(C₁₋₄-alkyl) (heteroaryl); N(C₁₋₄-alkyl)-C(═O)—N(C₁₋₄-alkyl)₂; N(C₁₋₄-alkyl)-C(═O)—N(C₁₋₄-alkyl)(C₃₋₆-cycloalkyl); N(C₁₋₄-alkyl)-C(═O)—N(C₁₋₄-alkyl)(3 to 7 membered heterocycloalkyl); N(C₁₋₄-alkyl)-C(═O)—N(C₁₋₄-alkyl)(aryl); N(C₁₋₄-alkyl)-C(═O)— N(C₁₋₄-alkyl) heteroaryl); SH; S—(C₁₋₄-alkyl); S—(C₃₋₆-cycloalkyl); S-(3 to 7 membered heterocycloalkyl); S-(aryl); S-(heteroaryl); SCF₃; S(═O)₂OH; S(═O)—(C₁₋₄-alkyl); S(═O)—(C₃₋₆-cycloalkyl); S(═O)-(3 to 7 membered heterocycloalkyl); S(═O)-(aryl); S(═O)-(heteroaryl); S(═O)₂—(C₁₋₄-alkyl); S(═O)₂—(C₃₋₆-cycloalkyl); S(═O)₂-(3 to 7 membered heterocycloalkyl); S(═O)₂-(aryl); S(═O)₂-(heteroaryl); S(═O)₂—O(C₁₋₄-alkyl); S(═O)₂—O(C₃₋₆-cycloalkyl); S(═O)₂—O(3 to 7 membered heterocycloalkyl); S(═O)₂—O(aryl); S(═O)₂—O(heteroaryl); S(═O)₂—N(H)(C₁₋₄-alkyl); S(═O)₂—N(H)(C₃₋₆-cycloalkyl); S(═O)₂—N(H)(3 to 7 membered heterocycloalkyl); S(═O)₂—N(H)(aryl); S(═O)₂—N(H)(heteroaryl); S(═O)₂—N(C₁₋₄-alkyl)₂; S(═O)₂—N(C₁₋₄-alkyl)(C₃₋₆-cycloalkyl); S(═O)₂—N(C₁₋₄-alkyl)(3 to 7 membered heterocycloalkyl); S(═O)₂—N(C₁₋₄-alkyl)(aryl); S(═O)₂—N(C₁₋₄-alkyl)(heteroaryl). The term “mono- or polysubstituted heteroaryl” refers in the sense of this invention to the corresponding N-oxides, if applicable,

Preferred substituents of “aryl” and “heteroaryl” are selected from the group consisting of F; Cl; Br; NO₂; CN; CF₃; CF₂H; CFH₂; CF₂Cl; CFCl₂; C₁₋₄-alkyl; aryl; heteroaryl; C₃₋₆-cycloalkyl; 3 to 6 membered heterocycloalkyl; aryl, heteroaryl, C₃₋₆-cycloalkyl or 3 to 6 membered heterocycloaliphatic, each connected via a C₁₋₄-aliphatic group; C(═O)—H; C(═O)—C₁₋₄-alkyl; C(═O)aryl; C(═O)heteroaryl; C(═O)—OH; C(═O)—O—C₁₋₄-alkyl; C(═O)O-aryl; C(═O)O-heteroaryl; CO—NH₂; C(═O)—N(H)C₁₋₄-alkyl; C(═O)—N(C₁₋₄-alkyl)₂; C(═O)NH-aryl; C(═O)N(aryl)₂; C(═O)NH-heteroaryl; C(═O)N(heteroaryl)₂; C(═O)N(C₁₋₄-alkyl)(aryl); C(═O)N(C₁₋₄-alkyl)(heteroaryl); C(═O)N(heteroaryl)(aryl); OH; OCF₃; OCF₂H; OCFH₂; OCF₂Cl; OCFCl₂; O—C₁₋₄-alkyl; O-benzyl; O-aryl; O-heteroaryl; O—C(═O)—C₁₋₄-alkyl; O—C(═O)aryl; O—C(═O)heteroaryl; O—C(═O)—O—C₁₋₄-alkyl; O—(C═O)—N(H)C₁₋₄-alkyl; O— C(═O)—N(C₁₋₄-alkyl)₂; O—S(═O)₂—C₁₋₄-alkyl; O—S(═O)₂—OH; O—S(═O)₂—O—C₁₋₄-alkyl; O—S(═O)₂—NH₂; O—S(═O)₂—N(H)C₁₋₄-alkyl; O—S(═O)₂—N(C₁₋₄-alkyl)₂; NH₂; N(H)C₁₋₄-alkyl; N(C₁₋₄-alkyl)₂; N(H)—C(═O)—C₁₋₄-alkyl; N(H)—C(═O)-aryl; N(H)—C(═O)-heteroaryl; N(H)—C(═O)—O—C₁₋₄-alkyl; N(H)C(═O)—NH₂; N(H)—C(═O)—N(H)C₁₋₄-alkyl; N(H)—C(═O)—N(C₁₋₄-alkyl)₂; N(C₁₋₄-alkyl)-C(═O)C₁₋₄-alkyl; N(C₁₋₄-alkyl)-C(═O)—O—C₁₋₄-alkyl; N(C₁₋₄-alkyl)C(═O)—NH₂; N(C₁₋₄-alkyl)-C(═O)—N(H)C₁₋₄-alkyl; N(C₁₋₄-alkyl)-C(═O)—N(C₁₋₄-alkyl)₂; N(H)—S(═O)₂—OH; N(H)—S(═O)₂—C₁₋₄-alkyl; N(H)—S(═O)₂—O—C₁₋₄-alkyl; N(H)—S(═O)₂—NH₂; N(H)S(═O)₂—N(H)C₁₋₄-alkyl; N(H)—S(═O)₂—N(C₁₋₄-alkyl)₂; N(C₁₋₄-alkyl)-S(═O)₂—OH; N(C₁₋₄-alkyl)-S(═O)₂(C₁₋₄-alkyl); N(C₁₋₄-alkyl)-S(═O)₂—O(C₁₋₄-alkyl); N(C₁₋₄-alkyl)-S(═O)₂—NH₂; N(C₁₋₄-alkyl)-S(═O)₂—N(H)C₁₋₄-alkyl; N(C₁₋₄-alkyl)S(═O)₂—N(C₁₋₄-alkyl)₂; SH; SCF₃; SCF₂H; SCFH₂; SCF₂Cl; SCFCl₂; S—C₁₋₄-alkyl; S-benzyl; S-aryl; Sheteroaryl; S(═O)—C₁₋₄-alkyl; S(═O)₂—C₁₋₄-alkyl; S(═O)₂-aryl; S(═O)₂-heteroaryl; S(═O)₂—OH; S(═O)₂—OC₁₋₄-alkyl; S(═O)₂O-aryl; S(═O)₂O-heteroaryl; S(═O)₂—NH₂; S(═O)₂—N(H)C₁₋₄-alkyl, S(═O)₂—N(H)-aryl; S(═O)₂—N(H)-heteroaryl and S(═O)₂—N(C₁₋₄-alkyl)₂.

More preferred substituents of “aryl” and “heteroaryl” are selected from the group consisting of F; Cl; CF₃; CN; C₁₋₄-alkyl; C(═O)—OH; C(═O)—O—C₁₋₄-alkyl; CO—NH₂; C(═O)—N(H)C₁₋₄-alkyl; C(═O)—N(C₁₋₄-alkyl)₂; OH; O—C₁₋₄-alkyl; O—C(═O)—C₁₋₄-alkyl; OCF₃; OCHF₂; OCH₂F; NH₂; N(H)C₁₋₄-alkyl; N(C₁₋₄-alkyl)₂; N(H)—C(═O)—C₁₋₄-alkyl; N(C₁₋₄-alkyl)-C(═O)C₁₋₄-alkyl; N(H)—S(═O)₂—C₁₋₄-alkyl; N(C₁₋₄-alkyl)-S(═O)₂(C₁₋₄-alkyl); N(H)C(═O)NH₂; N(H)—C(═O)—N(H)C₁₋₄-alkyl; N(H)—C(═O)—N(C₁₋₄-alkyl)₂; N(C₁₋₄-alkyl)-C(═O)—NH₂; N(C₁₋₄-alkyl)-C(═O)—N(H)C₁₋₄-alkyl; N(C₁₋₄-alkyl)-C(═O)—N(C₁₋₄-alkyl)₂; S(═O)₂C₁₋₄-alkyl; S(═O)₂—NH₂; S(═O)₂—N(H)C₁₋₄-alkyl and S(═O)₂—N(C₁₋₄-alkyl)₂.

The compounds according to the invention are defined by substituents, for example by R^{A}, R^{B} and R^{C} (1^{st} generation substituents) which are for their part if appropriate themselves substituted (2^{nd} generation substituents). Depending on the definition, these substituents of the substituents can for their part be resubstituted (3^{rd} generation substituents). If, for example, R^{A}=a C₁₋₄-alkyl (1^{st} generation substituent), then the C₁₋₄-alkyl can for its part be substituted, for example with a N(H)C₁₋₄-alkyl (2^{nd} generation substituent). This produces the functional group R^{A}=(C₁₋₄-alkyl-N(H)—C₁₋₄-alkyl). The N(H)—C₁₋₄-alkyl can then for its part be resubstituted, for example with Cl (3^{rd} generation substituent). Overall, this produces the functional group R^{A}=C₁₋₄-alkyl-N(H)—C₁₋₄-alkyl-Cl, wherein the C₁₋₄-alkyl of the N(H)C₁₋₄-alkyl is substituted by Cl.

However, in a preferred embodiment, the 3^{rd} generation substituents may not be resubstituted, i.e. there are then no 4^{th} generation substituents.

In another preferred embodiment, the 2^{nd} generation substituents may not be resubstituted, i.e. there are then not even any 3^{rd} generation substituents. In other words, in this embodiment, in the case of general formula (I), for example, the functional groups for R¹ to R³ can each if appropriate be substituted; however, the respective substituents may then for their part not be resubstituted.

If a residue occurs multiply within a molecule, then this residue can have respectively different meanings for various substituents: if, for example, both R^{A} and R^{B} denote a 3 to 7 membered heterocycloalkyl, then the 3 to 7 membered heterocycloalkyl can e.g. represent morpholinyl for R^{A} and can represent piperazinyl for R^{B}.

Within the scope of the present invention, the symbol

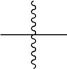

used in the formulae denotes a link of a corresponding residue to the respective superordinate general structure.

Surprisingly, it has been found that compounds according to claim 1 bearing a particular pyridinyl substitution pattern provide pronounced inhibition of the CRAC channels.

Another embodiment of the first aspect of the invention is a compound according to general formula (I) characterized in that the compound has general formula (Ia),

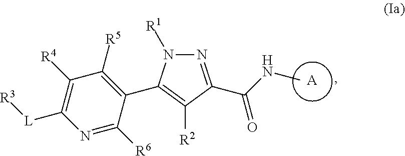

and R¹, R², R³, R⁴, R⁵, R⁶, L and A are defined as above.

In one embodiment of the invention, the compound according to the present invention is characterized in that R² is selected from the group consisting of H; F; Cl; Br; CN; CF₃; CF₂H; CFH₂; CH₃; CH₂CH₃; CH(CH₃)₂; cyclopropyl; OH; OCH₃; OCF₃; OCF₂H; OCFH₂; NH₂; N(H)CH₃; N(CH₃)₂.

Preferably, R² is selected from the group consisting of H, Cl, NH₂, CH₃ and CH₂CH₃. Most preferably, R² denotes H.

In another embodiment of the invention, the compound according to the present invention is characterized in that R¹ denotes H; C₁₋₄-alkyl, unsubstituted or mono- or polysubstituted or C₃₋₆-cycloalkyl, unsubstituted or mono- or polysubstituted.

Preferably, R¹ is selected from the group consisting of unsubstituted C₁₋₄-alkyl or unsubstituted cyclopropyl.

More preferably, R¹ is selected from the group consisting of unsubstituted C₁₋₄-alkyl. Even more preferably, R¹ is selected from CH₃ and CH₂CH₃. Most preferably, R¹ denotes CH₃.

In another embodiment of the invention, the compound according to the present invention is characterized in that A represents phenyl or 6-membered heteroaryl, each unsubstituted or mono- or polysubstituted.

Preferably, A represents phenyl or a 6-membered heteroaryl, containing one 1, 2 or 3 N-atoms, wherein said phenyl or 6-membered heteroaryl is unsubstituted or mono- or polysubstituted.

More preferably, A represents phenyl or a 6-membered heteroaryl, containing one 1, 2 or 3 N-atoms, wherein said phenyl or 6-membered heteroaryl is unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl).

Even more preferably, A is selected from the group consisting of phenyl, pyridinyl, pyrazinyl, pyrimidinyl, pyridazinyl, triazinyl, and the N-oxide of said pyridinyl, pyrazinyl, pyrimidinyl, pyridazinyl or triazinyl,

each unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl).

Still more preferably, A is phenyl or pyridinyl,

each unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl).

A particularly preferred embodiment of the first aspect of the invention is characterized by a certain substitution pattern of the structural element A to enhance affinity to the CRAC channel. Particularly preferred is therefore a compound according the first aspect of the invention, that is characterized in that A has substructure (II),

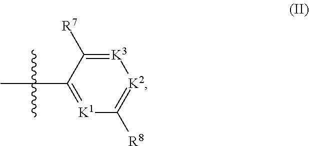

wherein
K¹ stands for N or CR⁷; K² stands for N or CR⁸ or N⁺—O⁻; and K³ stands for N or CR⁸;
each R⁷ independently is selected from H; F; Cl; Br; CN; CF₃; CF₂H; CFH₂; C₁₋₄-alkyl; C₃₋₆-cycloalkyl;
   and each R⁸ is independently selected from the group consisting of H; F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl).

More particularly preferred is a compound according the first aspect of the invention, that is characterized in that A has substructure (II), wherein K¹ stands for CR⁷; K² stands for CR⁸ and K³ stands CR⁸ or exactly one of K¹, K² and K³ stands for N.

More preferably, each R⁷ is independently selected from H; F; Cl; CF₃; CF₂H; CFH₂ and C₁₋₄-alkyl. Even more preferably, each R⁷ is independently selected from F; Cl and CH₃. Most preferably, each R⁷ is selected from F.

More preferably, each R⁸ is independently selected from the group consisting of H; F; Cl; CN; CF₃; C₁₋₄-alkyl; O—C₁₋₄-alkyl; OCF₃; OCF₂H or OCFH₂. Even more preferably, each R⁸ is independently selected from H; F; Cl; CF₃; OCF₃; CH₃ and OCH₃.

Another particularly preferred embodiment of the first aspect of the invention is characterized in that A is selected from the group consisting of 2,6-difluorophenyl, 5-fluoro-4-methyl-pyridin-3-yl, 3-fluoro-pyridin-4-yl, 2,4-dimethyl-pyridin-5-yl, 3,5-difluoro-pyridin-4-yl, 3,5-difluoro-pyridin-2-yl, 3,5-dichloro-pyridin-4-yl; 3-chloro-5-fluoro-pyridin-4-yl,3-fluoro-pyridin-2-yl, 4-fluoro-5-methyl-pyridin-3-yl, 2,6-difluoro-4-methoxyphenyl,2-chlorophenyl, 2-chloro-4-fluorophenyl, 2-chloro-6-fluorophenyl and 2,4-difluorophenyl.

In another embodiment of the first aspect of the invention,

the compound according to general formula (I) has general formula (Ia) and
R⁵ is selected from the group consisting H; F; Cl; CN; CF₃; CF₂H; CFH₂; C₁₋₄-alkyl; C₃₋₆-cycloalkyl; 3 to 7 membered heterocycloalkyl; O—C₁₋₄-alkyl; OCF₃; OCF₂H; OCFH₂; O—C₃₋₆-cycloalkyl; —O-(3 to 7 membered heterocycloalkyl), NH₂; N(H)C₁₋₄-alkyl; N(C₁₋₄-alkyl)₂; NH(C═O)(C₁₋₄-alkyl);
   wherein said C₁₋₄-alkyl is linear or branched, and is unsubstituted or mono- or polysubstituted;
   and wherein said C₃₋₆-cycloalkyl and 3 to 7 membered heterocycloalkyl is unsubstituted or mono- or polysubstituted.

Preferably, the compound according to general formula (I) has general formula (Ia) and

R⁵ is selected from the group consisting F; Cl; CN; CF₃; CF₂H; CFH₂; C₁₋₄-alkyl; C₃₋₆-cycloalkyl; 3 to 7 membered heterocycloalkyl; O—C₁₋₄-alkyl; OCF₃; OCF₂H; OCFH₂; O—C₃₋₆-cycloalkyl; O-(3 to 7 membered heterocycloalkyl),
wherein said C₁₋₄-alkyl is linear or branched, and is unsubstituted or mono- or polysubstituted;
and wherein said C₃₋₆-cycloalkyl and 3 to 7 membered heterocycloalkyl is unsubstituted or mono- or polysubstituted.

More preferably, the compound according to general formula (I) has general formula (Ia) and

R⁵ is selected from the group consisting O—C₁₋₄-alkyl; OCF₃; OCF₂H; OCFH₂; O—C₃₋₆-cycloalkyl and —O-(3 to 7 membered heterocycloalkyl),
wherein said C₁₋₄-alkyl is linear or branched, and is unsubstituted or mono- or polysubstituted;
and wherein said C₃₋₆-cycloalkyl and 3 to 7 membered heterocycloalkyl is unsubstituted or mono- or polysubstituted.

Particular preferably, the compound according to general formula (I) has general formula (Ia) and

R⁵ is selected from the group consisting
F; Cl; CN; CF₃; CH₃; CH₂CH₃; cyclopropyl; oxetanyl; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl; O-cyclohexyl and O-oxetanyl.

Even more preferably, the compound according to general formula (I) has general formula (Ia) and

R⁵ is selected from the group consisting CF₃; CH₃; CH₂CH₃; cyclopropyl; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl and O-cyclohexyl.

In another embodiment of the first aspect of the invention,

the compound according to general formula (I) is characterized in that R⁴ and R⁶ are independently selected from the group consisting H; F; Cl; CN; CF₃; CF₂H; CFH₂; C₁₋₄-alkyl; C₃₋₆-cycloalkyl; O—C₁₋₄-alkyl; OCF₃; OCF₂H and OCFH₂.

Preferably, the compound according to general formula (I) has general formula (Ia) and R⁴ and R⁶ are independently selected from the group consisting H; F; Cl; CN; CF₃; CF₂H; CFH₂; C₁₋₄-alkyl; C₃₋₆-cycloalkyl; O—C₁₋₄-alkyl; OCF₃; OCF₂H and OCFH₂.

More preferably, the compound according to general formula (I) has general formula (Ia) and R⁴ and R⁶ are independently selected from the group consisting H; F and CH₃. More preferably, R⁴ is selected from the group consisting H; F and CH₃ and R⁶ is H. Even more preferably, the compound according to general formula (I) has general formula (Ia) and R⁴ and R⁶ both represent H.

Even more preferably,

the compound according to general formula (I) has general formula (Ia);
R⁵ is selected from the group consisting CF₃; CH₃; CH₂CH₃; cyclopropyl; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl; O-cyclohexyl and O-oxetanyl;
and R⁴ and R⁶ are independently selected from the group consisting H; F and CH₃.

Still more preferably,

the compound according to general formula (I) has general formula (Ia);
R⁵ is selected from the group consisting CF₃; CH₃; CH₂CH₃; cyclopropyl; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl, O-cyclohexyl and O-oxetanyl;
and R⁴ and R⁶ denote H.

In another embodiment of the first aspect of the invention, the compound according to general formula (I) is characterized in that

L-R³ is selected from the group consisting of C₁₋₄-alkyl; C₃₋₆-cycloalkyl; -(3 to 7 membered heterocycloalkyl); O—C₁₋₄-alkyl; OCF₃; OCF₂H; —OCFH₂; O—C₃₋₆-cycloalkyl; O-(3 to 7 membered heterocycloalkyl); O—C₁₋₄-alkylene-C₃₋₆-cycloalkyl; O—C₁₋₄-alkylene-(3 to 7 membered heterocycloalkyl); O—C₁₋₄-alkylene-(5- to 6-membered heteroaryl); NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl) and N(C₁₋₄-alkyl)S(═O)₂(C₁₋₄-alkyl),
wherein said C₁₋₄-alkyl and C₁₋₄-alkylene is linear or branched, and is unsubstituted or mono- or polysubstituted;
and wherein said 5 to 6-membered heteroaryl, C₃₋₆-cycloalkyl and 3 to 7 membered heterocycloalky is unsubstituted or mono- or polysubstituted.

Preferably,

the compound according to general formula (I) has general formula (Ia) and
L-R³ is selected from the group consisting of C₁₋₄-alkyl; C₃₋₆-cycloalkyl; 3 to 7 membered heterocycloalkyl; O—C₁₋₄-alkyl; OCF₃; OCF₂H; OCFH₂; O—C₃₋₆-cycloalkyl; O-(3 to 7 membered heterocycloalkyl); O—C₁₋₄-alkylene-C₃₋₆-cycloalkyl; O—C₁₋₄-alkylene-(3 to 7 membered heterocycloalkyl); O—C₁₋₄-alkylene-(5 to 6-membered heteroaryl); NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl) and N(C₁₋₄-alkyl)S(═O)₂(C₁₋₄-alkyl),
wherein said C₁₋₄-alkyl and C₁₋₄-alkylene is linear or branched, and is unsubstituted or mono- or polysubstituted;
and wherein said 5 to 6-membered heteroaryl, C₃₋₆-cycloalkyl and 3 to 7 membered heterocycloalkyl is unsubstituted or mono- or polysubstituted.

Preferably,

L-R³ is selected from the group consisting of
C₁₋₄-alkyl; C₃₋₆-cycloalkyl; (3 to 7 membered heterocycloalkyl); O—C₁₋₄-alkyl; OCF₃; OCF₂H; OCFH₂; O—C₃₋₆-cycloalkyl; O-(3 to 7 membered heterocycloalkyl); O—C₁₋₄-alkylene-C₃₋₆-cycloalkyl; O—C₁₋₄-alkylene-(3 to 7 membered heterocycloalkyl); O—C₁₋₄-alkylene-(5 to 6-membered heteroaryl); O—C₁₋₄-alkylene-C(═O)OH; O—C₁₋₄-alkylene-C(═O)NH₂; O—C₁₋₄-alkylene-C(═O)N(H)C₁₋₄-alkyl; O—C₁₋₄-alkylene-C(═O)N(C₁₋₄-alkyl)₂; O—C₁₋₄-alkylene-OH; O—C₁₋₄-alkylene-O—C₁₋₄-alkyl; O—C₁₋₄-alkylene-OCF₃; O—C₁₋₄-alkylene-NH₂; O—C₁₋₄-alkylene-N(H)C₁₋₄-alkyl; O—C₁₋₄-alkylene-N(C₁₋₄-alkyl)₂; NH₂; N(H)C₁₋₄-alkyl and N(C₁₋₄-alkyl)₂,
wherein said C₁₋₄-alkyl and C₁₋₄-alkylene is linear or branched, and is unsubstituted or mono- or polysubstituted by F, Cl or OCH₃;
and wherein said 5 to 6-membered heteroaryl, C₃₋₆-cycloalkyl and 3 to 7 membered heterocycloalkyl is unsubstituted or mono- or polysubstituted by F, Cl, CH₃, CF₃ or OCH₃.

More preferably,

L-R³ is selected from the group consisting of
CH₃, CF₃, cyclopropyl; OCH₃; N(CH₃)₂; OCH₂CH₃; O(CH₂)₂CH₃; OCH(CH₃)₂; OCH₂CH(CH₃)₂; OC(CH₃)₃; OCF₃; OCHF₂; OCFH₂; OCH₂CF₃; O-cyclopropyl; O-cyclobutyl; OCH₂-cyclopropyl; OCH₂-cyclobutyl and O-(3-oxetanyl) and OCH₂-(3-oxetanyl).

Even more preferably,

the compound according to general formula (I) has general formula (Ia); L-R³ is selected from the group consisting of
CH₃, CF₃, cyclopropyl, OCH₃; N(CH₃)₂; OCH₂CH₃; O(CH₂)₂CH₃; OCH(CH₃)₂; OCH₂CH(CH₃)₂; OC(CH₃)₃; OCF₃; OCF₂H; OCH₂CF₃; O-cyclopropyl; O-cyclobutyl; OCH₂-cyclopropyl; OCH₂-cyclobutyl and O-(3-oxetanyl) and OCH₂-(3-oxetanyl);
R⁵ is selected from the group consisting CF₃; CH₃; CH₂CH₃; cyclopropyl; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl, O-cyclopentyl and O-cyclohexyl;
and R⁴ and R⁶ are independently selected from the group consisting of H; F and CH₃.

In yet another embodiment of the first aspect of the invention is characterized in that the compound according to general formula (I) is represented by general formula (Ia),

wherein
R¹ is selected from the group consisting of unsubstituted C₁₋₄-alkyl or unsubstituted cyclopropyl;
R² is selected from the group consisting of H, Cl, NH₂, CH₃ and CH₂CH₃;
R⁵ is selected from the group consisting of is selected from the group consisting of F; Cl; CN; CF₃; CF₂H; CFH₂; C₁₋₄-alkyl; C₃₋₆-cycloalkyl; 3 to 7 membered heterocycloalkyl; O—C₁₋₄-alkyl; OCF₃; OCF₂H; OCFH₂; O—C₃₋₆-cycloalkyl; O-(3 to 7 membered heterocycloalkyl), NH₂; N(H)C₁₋₄-alkyl; N(C₁₋₄-alkyl)₂ and NH(C═O)(C₁₋₄-alkyl);
   wherein said C₁₋₄-alkyl is linear or branched, and is unsubstituted or mono- or polysubstituted;
   and wherein said C₃₋₆-cycloalkyl and 3 to 7 membered heterocycloalkyl is unsubstituted or mono- or polysubstituted;
R⁴ and R⁶ are independently selected from the group consisting of H; F and CH₃;
L-R³ is selected from the group consisting of
   C₁₋₄-alkyl; C₃₋₆-cycloalkyl; (3 to 7 membered heterocycloalkyl); O—C₁₋₄-alkyl; OCF₃; OCF₂H; OCFH₂; O—C₃₋₆-cycloalkyl; O-(3 to 7 membered heterocycloalkyl); O—C₁₋₄-alkylene-C₃₋₆-cycloalkyl; O—C₁₋₄-alkylene-(3 to 7 membered heterocycloalkyl); O—C₁₋₄-alkylene-(5 to 6-membered heteroaryl); O—C₁₋₄-alkylene-C(═O)OH; O—C₁₋₄-alkylene-C(═O)NH₂; O—C₁₋₄-alkylene-C(═O)N(H)C₁₋₄-alkyl; O—C₁₋₄-alkylene-C(═O)N(C₁₋₄-alkyl)₂; O—C₁₋₄-alkylene-OH; O—C₁₋₄-alkylene-O—C₁₋₄-alkyl; O—C₁₋₄-alkylene-OCF₃; O—C₁₋₄-alkylene-NH₂; O—C₁₋₄-alkylene-N(H)C₁₋₄-alkyl; O—C₁₋₄-alkylene-N(C₁₋₄-alkyl)₂; NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl) and N(C₁₋₄-alkyl)S(═O)₂(C₁₋₄-alkyl),
   wherein said C₁₋₄-alkyl and C₁₋₄-alkylene is linear or branched, and is unsubstituted or mono- or polysubstituted by F, Cl or OCH₃;
   and wherein said 5 to 6-membered heteroaryl, C₃₋₆-cycloalkyl and 3 to 7 membered heterocycloalkyl is unsubstituted or mono- or polysubstituted by F, Cl, CH₃, CF₃ or OCH₃;
      and
A is selected from the group consisting of phenyl and pyridinyl,
   each unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl).

Preferably, the compound according to general formula (I) is represented by general formula (Ia), wherein
R¹ denotes CH₃;
R² denotes H;
R⁵ is selected from the group consisting of F; Cl; CN; CF₃; CF₂H; CFH₂; C₁₋₄-alkyl; C₃₋₆-cycloalkyl; 3 to 7 membered heterocycloalkyl; O—C₁₋₄-alkyl; OCF₃; OCF₂H; OCFH₂; O—C₃₋₆-cycloalkyl; —O-(3 to 7 membered heterocycloalkyl),
   wherein said C₁₋₄-alkyl is linear or branched, and is unsubstituted or mono- or polysubstituted;
   and wherein said C₃₋₆-cycloalkyl and 3 to 7 membered heterocycloalkyl is unsubstituted or mono- or polysubstituted.
R⁴ and R⁶ are independently selected from the group consisting of H; F and CH₃;
L-R³ is selected from the group consisting of
   C₁₋₄-alkyl; C₃₋₆-cycloalkyl; (3 to 7 membered heterocycloalkyl); O—C₁₋₄-alkyl; OCF₃; OCF₂H; OCFH₂; O—C₃₋₆-cycloalkyl; O-(3 to 7 membered heterocycloalkyl); O—C₁₋₄-alkylene-C₃₋₆-cycloalkyl; O—C₁₋₄-alkylene-(3 to 7 membered heterocycloalkyl); O—C₁₋₄-alkylene-(5 to 6-membered heteroaryl); O—C₁₋₄-alkylene-C(═O)OH; O—C₁₋₄-alkylene-C(═O)NH₂; O—C₁₋₄-alkylene-C(═O)N(H)C₁₋₄-alkyl; O—C₁₋₄-alkylene-C(═O)N(C₁₋₄-alkyl); O—C₁₋₄-alkylene-OH; O—C₁₋₄-alkylene-O—C₁₋₄-alkyl; O—C₁₋₄-alkylene-OCF₃; O—C₁₋₄-alkylene-NH₂; O—C₁₋₄-alkylene-N(H)C₁₋₄-alkyl; O—C₁₋₄-alkylene-N(C₁₋₄-alkyl); NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl) and N(C₁₋₄-alkyl)S(═O)₂(C₁₋₄-alkyl),
   wherein said C₁₋₄-alkyl and C₁₋₄-alkylene is linear or branched, and is unsubstituted or mono- or polysubstituted by F, Cl or OCH₃;
   and wherein said 5 to 6-membered heteroaryl, C₃₋₆-cycloalkyl and 3 to 7 membered heterocycloalkyl is unsubstituted or mono- or polysubstituted by F, Cl, CH₃, CF₃ or OCH₃;
and
A is selected from the group consisting of 2,6-difluorophenyl, 5-fluoro-4-methyl-pyridin-3-yl, 3-fluoro-pyridin-4-yl, 2,4-dimethyl-pyridin-5-yl, 3,5-difluoro-pyridin-4-yl, 3,5-difluoro-pyridin-2-yl, 3,5-dichloro-pyridin-4-yl; 3-chloro-5-fluoro-pyridin-4-yl,3-fluoro-pyridin-2-yl, 4-fluoro-5-methyl-pyridin-3-yl, 2,6-difluoro-4-methoxyphenyl, 2-chlorophenyl, 2-chloro-4-fluorophenyl, 2-chloro-6-fluorophenyl and 2,4-difluorophenyl.

In yet another embodiment of the first aspect of the invention is characterized in that the compound according to general formula (I) is represented by general formula (Ia),

wherein
R¹ denotes CH₃;
R² denotes H;
R⁵ is selected from the group consisting of CF₃; CH₃; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl; O-cyclohexyl and O-oxetanyl;
R⁴ and R⁶ denote H;
L-R³ is selected from the group consisting of CH₃; CF₃; cyclopropyl; OCH₃; N(CH₃)₂; OCH₂CH₃; O(CH₂)₂CH₃; OCH(CH₃)₂; OCH₂CH(CH₃)₂; OC(CH₃)₃; OCF₃; OCF₂H; OCFH₂; OCH₂CF₃; O-cyclopropyl; O-cyclobutyl; OCH₂-cyclopropyl; OCH₂-cyclobutyl; O-(3-oxetanyl) and OCH₂-(3-oxetanyl);
   and
A is selected from the group consisting of phenyl and pyridinyl,
   each unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl).

More preferably, the compound according to general formula (I) is represented by general formula (Ia), wherein
R¹ denotes CH₃;
R² denotes H;
R⁵ is selected from the group consisting of CF₃; CH₃; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl; O-cyclohexyl and O-oxetanyl;
R⁴ and R⁶ denote H;
L-R³ is selected from the group consisting of CH₃; CF₃; cyclopropyl; OCH₃; N(CH₃)₂; OCH₂CH₃; O(CH₂)₂CH₃; OCH(CH₃)₂; OCH₂CH(CH₃)₂; OC(CH₃)₃; OCF₃; OCF₂H; OCFH₂; OCH₂CF₃; O-cyclopropyl; O-cyclobutyl; OCH₂-cyclopropyl; OCH₂-cyclobutyl; O-(3-oxetanyl) and OCH₂-(3-oxetanyl);
   and
A is selected from the group consisting of 2,6-difluorophenyl, 3,5-difluoro-pyridin-4-yl, 3,5-dichloro-pyridin-4-yl; 3-chloro-5-fluoro-pyridin-4-yl and 3-fluoro-pyridin-2-yl.

A preferred embodiment of the first aspect of the invention is a compound according to general formula (I) characterized in that the compound has general formula (Ib):

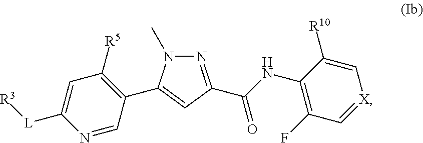

wherein R¹⁰ is H or F or Cl;
X is N or CH;
R⁵ is selected from the group consisting of CF₃; CH₃; OCH₃; OCF₃; OCF₂H and OCFH₂; and
L-R³ is selected from the group consisting of CH₃; CF₃; cyclopropyl; OCH₃; N(CH₃)₂;

OCH₂CH₃; O(CH₂)₂CH₃; OCH(CH₃)₂; OCH₂CH(CH₃)₂; OC(CH₃)₃; OCF₃; OCF₂H; OCFH₂; OCH₂CF₃; O-cyclopropyl; O-cyclobutyl; OCH₂-cyclopropyl; OCH₂-cyclobutyl; O-(3-oxetanyl) and OCH₂-(3-oxetanyl).

In a particular preferred embodiment of the first embodiment of the present invention, the compound according to the present invention is selected from the group, consisting of
1 5-(6-Ethoxy-4-methyl-pyridin-3-yl)-N-(3-fluoro-pyridin-4-yl)-1-methyl-1H-pyrazole-3-carboxylic acid amide;
2 N-(3,5-Difluoro-pyridin-4-yl)-5-(6-ethoxy-4-methyl-pyridin-3-yl)-1-methyl-1H-pyrazole-3-carboxylic acid amide;
3 N-(2,6-Difluoro-phenyl)-5-(6-ethoxy-4-methyl-pyridin-3-yl)-1-methyl-1H-pyrazole-3-carboxylic acid amide;
4 N-(3,5-Difluoro-pyridin-4-yl)-5-(6-ethoxy-2-methyl-pyridin-3-yl)-1-methyl-1H-pyrazole-3-carboxylic acid amide;
5 N-(2,6-Difluoro-phenyl)-5-(6-ethoxy-2-methyl-pyridin-3-yl)-1-methyl-1H-pyrazole-3-carboxylic acid amide;
6 N-(2,6-Difluoro-phenyl)-5-(6-ethyl-4-methyl-pyridin-3-yl)-1-methyl-1H-pyrazole-3-carboxylic acid amide;
7 N-(3,5-Difluoro-pyridin-4-yl)-5-(6-ethyl-4-methyl-pyridin-3-yl)-1-methyl-1H-pyrazole-3-carboxylic acid amide;
8 5-(6-Cyclopropyl-4-methyl-pyridin-3-yl)-N-(2,6-difluoro-phenyl)-1-methyl-1H-pyrazole-3-carboxylic acid amide;
9 5-(6-Cyclopropyloxy-4-methyl-pyridin-3-yl)-N-(3,5-difluoro-pyridin-4-yl)-1-methyl-1H-pyrazole-3-carboxylic acid amide;
10 5-(6-Cyclopropyloxy-4-methyl-pyridin-3-yl)-N-(2,6-difluoro-phenyl)-1-methyl-1H-pyrazole-3-carboxylic acid amide;
11 N-(3,5-Difluoro-pyridin-4-yl)-1-methyl-5-[4-methyl-6-(trifluoromethyloxy)-pyridin-3-yl]-1H-pyrazole-3-carboxylic acid amide;
12 N-(2,6-Difluoro-phenyl)-1-methyl-5-[4-methyl-6-(trifluoromethyloxy)-pyridin-3-yl]-1H-pyrazole-3-carboxylic acid amide;
13 N-(2,6-Difluoro-phenyl)-5-(6-isopropoxy-4-methyl-pyridin-3-yl)-1-methyl-1H-pyrazole-3-carboxylic acid amide;
14 N-(3,5-Difluoro-pyridin-4-yl)-5-(6-ethoxy-4-methoxy-pyridin-3-yl)-1-methyl-1H-pyrazole-3-carboxylic acid amide;
15 5-(4,6-Diethoxy-pyridin-3-yl)-N-(3,5-difluoro-pyridin-4-yl)-1-methyl-1H-pyrazole-3-carboxylic acid amide;
16 N-(2,6-Difluoro-phenyl)-5-(6-ethoxy-4-methoxy-pyridin-3-yl)-1-methyl-1H-pyrazole-3-carboxylic acid amide;
17 5-(4,6-Diethoxy-pyridin-3-yl)-N-(2,6-difluoro-phenyl)-1-methyl-1H-pyrazole-3-carboxylic acid amide;
18 5-(6-Cyclopropyloxy-4-methyl-pyridin-3-yl)-N-(3,5-difluoro-pyridin-4-yl)-1-methyl-1H-pyrazole-3-carboxylic acid amide;
19 -(2,6-Difluoro-phenyl)-1-methyl-5-[4-methyl-6-[(5-methyl-isoxazol-3-yl)-methoxy]-pyridin-3-yl]-1H-pyrazole-3-carboxylic acid amide;
20 5-[6-(Cyclobutyl-methoxy)-4-methyl-pyridin-3-yl]-N-(3,5-difluoro-pyridin-4-yl)-1-methyl-1H-pyrazole-3-carboxylic acid amide;
21 N-(2,6-Difluoro-phenyl)-5-[6-[2-(dimethylamino)ethyloxy]-4-methyl-pyridin-3-yl]-1-methyl-1H-pyrazole-3-carboxylic acid amide;
22 5-(6-Chloro-4-methyl-pyridin-3-yl)-N-(2,6-difluoro-phenyl)-1-methyl-1H-pyrazole-3-carboxylic acid amide;
23 N-(2,6-Difluoro-phenyl)-1-methyl-5-[4-methyl-6-(oxetan-3-yloxy)-pyridin-3-yl]-1H-pyrazole-3-carboxylic acid amide;
24 N-(2,6-Difluoro-phenyl)-5-(6-hydroxy-4-methyl-pyridin-3-yl)-1-methyl-1H-pyrazole-3-carboxylic acid amide;
25 N-(3,5-Difluoro-pyridin-4-yl)-5-(6-hydroxy-4-methyl-pyridin-3-yl)-1-methyl-1H-pyrazole-3-carboxylic acid amide;
26 2-[5-[5-[(2,6-Difluoro-phenyl)-carbamoyl]-2-methyl-2H-pyrazol-3-yl]-4-methyl-pyridin-2-yl]oxy-acetic acid ethyl ester;
27 2-[5-[5-[(2,6-Difluoro-phenyl)-carbamoyl]-2-methyl-2H-pyrazol-3-yl]-4-methyl-pyridin-2-yl]oxy-acetic acid;
28 N-(2,6-Difluoro-phenyl)-5-[6-(2-methoxy-ethoxy)-4-methyl-pyridin-3-yl]-1-methyl-1H-pyrazole-3-carboxylic acid amide;
29-(2,6-Difluoro-phenyl)-5-[6-[(dimethyl-carbamoylymethoxy]-4-methyl-pyridin-3-yl]-1-methyl-1H-pyrazole-3-carboxylic acid amide;
30 5-[6-(Carbamoyl-methoxy)-4-methyl-pyridin-3-yl]-N-(2,6-difluoro-phenyl)-1-methyl-1H-pyrazole-3-carboxylic acid amide;
31 N-(3,5-Difluoro-pyridin-4-yl)-5-[6-ethoxy-4-(trifluoromethyl)-pyridin-3-yl]-1-methyl-1H-pyrazole-3-carboxylic acid amide;
32 N-(2,6-Difluoro-phenyl)-5-[6-ethoxy-4-(trifluoromethyl)-pyridin-3-yl]-1-methyl-1H-pyrazole-3-carboxylic acid amide;
33 5-(6-Cyclopropyl-5-fluoro-4-methyl-pyridin-3-yl)-N-(2,6-difluoro-phenyl)-1-methyl-1H-pyrazole-3-carboxylic acid amide;
34 5-(6-Cyclopropyl-5-fluoro-4-methyl-pyridin-3-yl)-N-(3,5-difluoro-pyridin-4-yl)-1-methyl-1H-pyrazole-3-carboxylic acid amide;
35 N-(2,6-Difluoro-phenyl)-5-(6-ethoxy-5-fluoro-4-methyl-pyridin-3-yl)-1-methyl-1H-pyrazole-3-carboxylic acid amide;
36 N-(3,5-Difluoro-pyridin-4-yl)-5-(6-ethoxy-5-fluoro-4-methyl-pyridin-3-yl)-1-methyl-1H-pyrazole-3-carboxylic acid amide;
37 N-(2,6-Difluoro-phenyl)-5-(6-dimethylamino-4-methyl-pyridin-3-yl)-1-methyl-1H-pyrazole-3-carboxylic acid amide;
38 N-(2,6-Difluoro-phenyl)-5-(4,6-dimethoxy-pyridin-3-yl)-1-methyl-1H-pyrazole-3-carboxylic acid amide;
39 5-(5-Chloro-2-methyl-pyridin-3-yl)-N-(3-fluoro-pyridin-4-yl)-1-methyl-1H-pyrazole-3-carboxylic acid amide;
40 5-(5-Chloro-2-methyl-pyridin-3-yl)-N-(2,6-difluoro-phenyl)-1-methyl-1H-pyrazole-3-carboxylic acid amide;
41 5-(6-Cyano-4-methyl-pyridin-3-yl)-N-(2,6-difluoro-phenyl)-1-methyl-1H-pyrazole-3-carboxylic acid amide;
42 N-(2,6-Difluoro-phenyl)-5-[4-methoxy-6-(trifluoromethyl)-pyridin-3-yl]-1-methyl-1H-pyrazole-3-carboxylic acid amide;
43 5-[6-(Difluoro-methoxy)-4-methoxy-pyridin-3-yl]-N-(2,6-difluoro-phenyl)-1-methyl-1H-pyrazole-3-carboxylic acid amide;
44 5-[6-(Difluoro-methoxy)-4-methoxy-pyridin-3-yl]-N-(3,5-difluoro-pyridin-4-yl)-1-methyl-1H-pyrazole-3-carboxylic acid amide;
45 5-[6-(Cyclopropyl-methoxy)-4-methoxy-pyridin-3-yl]-N-(2,6-difluoro-phenyl)-1-methyl-1H-pyrazole-3-carboxylic acid amide;
46 N-(2,6-Difluoro-phenyl)-5-[4-methoxy-6-(2,2,2-trifluoro-ethoxy)-pyridin-3-yl]-1-methyl-1H-pyrazole-3-carboxylic acid amide;
47 5-[4-(Difluoro-methoxy)-6-ethoxy-pyridin-3-yl]-N-(2,6-difluoro-phenyl)-1-methyl-1H-pyrazole-3-carboxylic acid amide;
48 5-[6-Cyclopropyl-4-(trifluoromethyl)-pyridin-3-yl]-N-(2,6-difluoro-phenyl)-1-methyl-1H-pyrazole-3-carboxylic acid amide;
49 N-(2,6-Difluoro-phenyl)-5-[6-ethoxy-4-(oxetan-3-yloxy)-pyridin-3-yl]-1-methyl-1H-pyrazole-3-carboxylic acid amide;
50 5-(6-Chloro-4-methoxy-pyridin-3-yl)-N-(2,6-difluoro-phenyl)-1-methyl-1H-pyrazole-3-carboxylic acid amide;
51 N-(2,6-Difluoro-phenyl)-5-[4-methoxy-6-(oxetan-3-yl-methoxy)-pyridin-3-yl]-1-methyl-1H-pyrazole-3-carboxylic acid amide;
52 N-(3,5-Difluoro-pyridin-4-yl)-5-(6-isopropoxy-4-methyl-pyridin-3-yl)-1-methyl-1H-pyrazole-3-carboxylic acid amide;
53 5-(6-Cyclopropyl-4-methoxy-pyridin-3-yl)-N-(3,5-difluoro-pyridin-4-yl)-1-methyl-1H-pyrazole-3-carboxylic acid amide;
54 5-(6-Cyclopropyl-4-methoxy-pyridin-3-yl)-N-(2,6-difluoro-phenyl)-1-methyl-1H-pyrazole-3-carboxylic acid amide;
55 5-[6-(Cyclobutyl-methoxy)-4-methoxy-pyridin-3-yl]-N-(2,6-difluoro-phenyl)-1-methyl-1H-pyrazole-3-carboxylic acid amide;
56 5-(4-Chloro-6-methoxy-pyridin-3-yl)-N-(2,6-difluoro-phenyl)-1-methyl-1H-pyrazole-3-carboxylic acid amide;
57 4-Chloro-N-(2,6-difluoro-phenyl)-5-(4,6-dimethoxy-pyridin-3-yl)-1-methyl-1H-pyrazole-3-carboxylic acid amide;
58 N-(2,6-Difluoro-phenyl)-5-(6-methoxy-4-methyl-pyridin-3-yl)-1-methyl-1H-pyrazole-3-carboxylic acid amide;
59 N-(2,6-Difluoro-phenyl)-5-[6-(methoxymethyl)-4-methyl-pyridin-3-yl]-1-methyl-1H-pyrazole-3-carboxylic acid amide;
60 N-(2,6-Difluoro-phenyl)-1-methyl-5-[4-methyl-6-(trifluoromethyl)-pyridin-3-yl]-1H-pyrazole-3-carboxylic acid öamide;
61 5-(6-Cyclopropyloxy-4-methoxy-pyridin-3-yl)-N-(2,6-difluoro-phenyl)-1-methyl-1H-pyrazole-3-carboxylic acid amide;
62 N-(2,6-Difluoro-phenyl)-5-[4-methoxy-6-(oxetan-3-yloxy)-pyridin-3-yl]-1-methyl-1H-pyrazole-3-carboxylic acid amide;
63 5-[6-Cyclopropyl-4-(trifluoromethyl)-pyridin-3-yl]-N-(3,5-difluoro-pyridin-4-yl)-1-methyl-1H-pyrazole-3-carboxylic acid amide;
64 N-(2,6-Difluoro-phenyl)-5-(4,6-dimethoxy-pyridin-3-yl)-1,4-dimethyl-1H-pyrazole-3-carboxylic acid amide;
65 N-(3,5-Difluoro-pyridin-4-yl)-5-(4,6-dimethoxy-pyridin-3-yl)-1,4-dimethyl-1H-pyrazole-3-carboxylic acid amide;
66 5-(4,6-Dimethoxy-pyridin-3-yl)-N-(4,6-dimethyl-pyridin-3-yl)-1,4-dimethyl-1H-pyrazole-3-carboxylic acid amide;
67 5-(4,6-Dimethoxy-pyridin-3-yl)-N-(5-fluoro-4-methyl-pyridin-3-yl)-1,4-di methyl-1H-pyrazole-3-carboxylic acid amide;
68 N-(2,6-Difluoro-phenyl)-5-(6-ethoxy-4-methoxy-pyridin-3-yl)-1H-pyrazole-3-carboxylic acid amide;
69 N-(2,6-Difluoro-phenyl)-5-(4,6-dimethoxy-pyridin-3-yl)-1H-pyrazole-3-carboxylic acid amide;
70 N-(2,6-Difluoro-phenyl)-5-(6-hydroxy-4-methoxy-pyridin-3-yl)-1-methyl-1H-pyrazole-3-carboxylic acid amide;
71 N-(2,6-Difluoro-phenyl)-5-(6-ethoxy-4-hydroxy-pyridin-3-yl)-1-methyl-1H-pyrazole-3-carboxylic acid amide
72 5-(3-Cyano-6-trifluoromethyl-pyridin-2-yl)-N-(2,6-difluoro-phenyl)-1-methyl-1H-pyrazole-3-carboxylic acid amide;
73 5-(6-tert-Butyl-3-cyano-pyridin-2-yl)-N-(2,6-difluoro-phenyl)-1-methyl-1H-pyrazole-3-carboxylic acid amide;
74 N-(2,6-Difluoro-phenyl)-5-[2-methoxy-5-(4-methyl-5-oxo-4H-[1,3,4]oxadiazol-2-yl)-pyridin-3-yl]-1-methyl-1H-pyrazole-3-carboxylic acid amide;
75 N-(2,6-Difluoro-phenyl)-5-(4-dimethylamino-6-methoxy-pyridin-3-yl)-1-methyl-1H-pyrazole-3-carboxylic acid amide;
76 5-(6-Ethoxy-4-methyl-pyridin-3-yl)-N-(3-fluoro-5-methyl-pyridin-4-yl)-1-methyl-1H-pyrazole-3-carboxylic acid amide;
77 5-(6-Cyclopropyl-4-methyl-pyridin-3-yl)-N-(3-fluoro-5-methyl-pyridin-4-yl)-1-methyl-1H-pyrazole-3-carboxylic acid amide;
78 N-(2,6-Difluoro-phenyl)-5-(4-ethoxy-6-methoxy-pyridin-3-yl)-1-methyl-1H-pyrazole-3-carboxylic acid amide;
79 N-(3,5-Difluoro-pyridin-4-yl)-5-(4,6-dimethoxy-pyridin-3-yl)-1-methyl-1H-pyrazole-3-carboxylic acid amide;
80 5-(4,6-Dimethoxy-pyridin-3-yl)-N-(5-fluoro-4-methyl-pyridin-3-yl)-1-methyl-1H-pyrazole-3-carboxylic acid amide;
81 5-(4,6-Dimethoxy-pyridin-3-yl)-N-(4,6-dimethyl-pyridin-3-yl)-1-methyl-1H-pyrazole-3-carboxylic acid amide;
82 4-Chloro-N-(3,5-difluoro-pyridin-4-yl)-5-(4,6-dimethoxy-pyridin-3-yl)-1-methyl-1H-pyrazole-3-carboxylic acid amide;
83 4-Chloro-5-(4,6-dimethoxy-pyridin-3-yl)-N-(5-fluoro-4-methyl-pyridin-3-yl)-1-methyl-1H-pyrazole-3-carboxylic acid amide;
84 4-Chloro-5-(4,6-dimethoxy-pyridin-3-yl)-N-(4,6-dimethyl-pyridin-3-yl)-1-methyl-1H-pyrazole-3-carboxylic acid amide;
85 N-(3,5-Difluoro-1-oxo-pyridin-4-yl)-5-(4,6-dimethoxy-pyridin-3-yl)-1-methyl-1H-pyrazole-3-carboxylic acid amide;
86 5-(4-Cyano-6-methoxy-pyridin-3-yl)-N-(2,6-difluoro-phenyl)-1-methyl-1H-pyrazole-3-carboxylic acid amide;
87 N-(2,6-Difluoro-phenyl)-5-(6-methoxy-4-methylamino-pyridin-3-yl)-1-methyl-1H-pyrazole-3-carboxylic acid amide;
88 N-(2,6-Difluoro-phenyl)-5-(4-hydroxy-6-methoxy-pyridin-3-yl)-1-methyl-1H-pyrazole-3-carboxylic acid amide;
   optionally in the form of the free compound and/or a physiologically acceptable salt thereof and/or a physiologically acceptable solvate thereof.

The compounds according to the present invention are useful for calcium release-activated calcium (CRAC) channel regulation, preferably for use in CRAC channel inhibition.

The substances according to the invention hence act, for example, on the CRAC channel relevant in connection with various diseases, so that they are suitable as a pharmacologically active compound in pharmaceutical compositions.

The compounds according to the first aspect of the present invention and the corresponding stereoisomers and the respective salts and solvates are toxicologically safe and are therefore suitable as pharmacologically active ingredients in pharmaceutical compositions.

In another aspect of the present invention, the invention therefore also provides pharmaceutical compositions, containing at least one compound according to the invention and optionally one or more suitable, pharmaceutically compatible auxiliaries and/or, if appropriate, one or more further pharmacologically active compounds.

The pharmaceutical composition according to the invention is suitable for administration to adults and children, including toddlers and babies.

The pharmaceutical composition according to the invention may be found as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one compound according to the invention, if appropriate in the form of one of its pure stereoisomers, in particular enantiomers or diastereomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally contains further physiologically compatible pharmaceutical auxiliaries which can for example be selected from the group consisting of excipients, fillers, solvents, diluents, surface-active substances, dyes, preservatives, blasting agents, slip additives, lubricants, aromas and binders. Likewise the compound according to the invention, if appropriate in the form of one of its pure stereoisomers, or if appropriate in the form of a corresponding salt or respectively in the form of a corresponding solvate, may also incorporated into the pharmaceutical composition in the form of a prodrug, which releases the active pharmacological agent through normal metabolic processes.

The selection of the physiologically compatible auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, drages, capsules, granules, pellets, drops, juices and syrups are preferably suitable for oral application; solutions, suspensions, easily reconstitutable dry preparations and also sprays are preferably suitable for parenteral, topical and inhalative application. The compounds according to the invention used in the pharmaceutical composition according to the invention in a repository in dissolved form or in a plaster, agents promoting skin penetration being added if appropriate, are suitable percutaneous application preparations. Orally or percutaneously applicable preparation forms can release the respective compound according to the invention also in a delayed manner.

The pharmaceutical compositions according to the invention are prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula I may vary and is for example dependent on the patient's weight or age and also on the type of application, the indication and the severity of the disorder. Conventionally 0.001 to 100 mg/kg, preferably 0.05 to 75 mg/kg, particularly preferably 0.05 to 50 mg of at least one such compound according to the invention are applied per kg of the patient's body weight.

CRAC channels are believed to be involved in a variety of diseases or disorders in mammals such as humans. These include inflammatory disorders, allergic disorders and disorders of the immune system as well as disorders involving platelet or thrombotic activity.

Examples of allergic disorders include: rhinitis (such as allergic rhinitis), sinusitis, rhinosinusitis, chronic or recurrent otitis media, drug reactions, insect sting reactions, latex allergy, conjunctivitis, urticaria, anaphylaxis and anaphylactoid reactions, atopic dermatitis and food allergies.

Examples of inflammatory disorders include: inflammatory lung disorders (such as asthma, acute respiratory distress syndrome, acute lung injury, chronic obstructive pulmonary disease, bronchiectasis and cystic fibrosis); chronic inflammatory disorders of joints (such as arthritis, rheumatoid arthritis, osteoarthritis and bone diseases associated with increased bone resorption); inflammatory bowel diseases (such as Barrett's oesophagus, ileitis, ulcerative colitis and Crohn's disease); inflammatory disorders of the eye (such as corneal dystrophy, trachoma, uveitis, sympathetic ophthalmitis and endo-phthalmitis); inflammatory diseases of the kidney (such as glomerulonephritis, nephrosis, nephritic syndrome and IgA nephropathy); inflammatory diseases of the liver; inflammatory disorders of the skin (such as psoriasis and eczema); inflammatory diseases of the central nervous system (such as chronic demyelinating diseases of the nervous system, multiple sclerosis, AIDS-related neurodegeneration and Alzheimers disease, infectious meningitis, enceophalomyelitis, Parkinson's disease, Huntington's disease, amyotrophic lateral sclerosis and viral or autoimmune encephalitis); inflammatory diseases of the muscle (such as polymyositis and polymyalgia rheumatica); inflammatory diseases of the heart (such as myocarditis and cardiomyopathy, ischemic heart disease, myocardial infarction and atherosclerosis); other diseases with significant inflammatory components, including tuberculosis; leprosy; allogeneic or xenogeneic transplantation (cells, stem cells, tissues or organs) graft rejection, graft-versus-host disease; pre-eclampsia; endometriosis, chronic liver failure; brain and spinal cord trauma and cancer; and conditions where systemic inflammation of the body may also be present (such as septic shock, hemorrhagic or anaphylactic shock or shock induced by cancer chemotherapy).

Examples of disorders of the immune system include: autoimmune diseases of the central and peripheral nervous system (such as multiple sclerosis, myasthenia gravis, Eaton-Lambert Myasthenic syndrome); autoimmune neurophathies (such as Guillain-Barre); autoimmune diseases of the eye (such as autoimmune uveitis); autoimmune diseases of the blood (such as autoimmune haemolytic anemia, pernicious anemia, and autoimmune thrombocytopenia e.g. Idiopathic Thrombocytopaenic Purpura); autoimmune diseases of the vasculature (such as temporal arteritis, anti-phospholipid syndrome, vasculitides e.g. Wegener's granulomatosis and Behcet's disease); autoimmune diseases of the skin (such as alopecia areata, psoriasis, dermatitis herpetiformis, pemphigus vulgaris, bullous pemphigoid and vitiligo); autoimmune disease of the gastrointestinal tract (such as coeliac disease, Crohn's disease, ulcerative colitis, primary biliary cirrhosis and autoimmune hepatitis); autoimmune disorders of the endocrine glands (such as Type1 diabetes mellitus, autoimmune thyroiditis, Grave's disease, Hashimoto's thyroiditis, autoimmune oophoritis and orchitis); autoimmune disorder of the adrenal gland (such as Addisons disease); autoimmune disorders of the exocrine glands (such as Sjogren's syndrome); and multi system autoimmune diseases including connective tissue and musculoskeletal system diseases (such as rheumatoid arthritis, systemic lupus erythematosus, scleroderma, polymyositis, dermatomyositis), spondyloarthropathies (such as ankylosing spondylitis and psoriatic arthritis).

Examples of conditions where anti-platelet or anti-thrombotic activity is useful for treatment and/or prophylaxis include: ischemic heart disease, myocardial infarction, cerebrovascular accident (stroke) and vascular thrombosis (venous, arterial and intra-cardiac).

Further diseases or conditions which may be treated by the compounds of the invention include conditions where mast cells and basophils contribute to pathology, such as mast cell leukaemia, mastocytosis, endometriosis and basophil leukaemia.

The term “disorders and/or diseases which are mediated, at least in part, by CRAC channels”, is intended to include each of or all of the above disease states.

It is believed that the compounds of formula (I), having ICRAC inhibitory activity, may inhibit mast cell degranulation and/or inhibit T cell activation. Compounds having such activity may be particularly suitable for the treatment of a number of diseases and conditions, for example asthma; allergies such as allergic rhinitis; and nasal polyposis.

Due to the key role of calcium in the regulation of cellular proliferation in general, calcium channel inhibitors could act as cytostatic agents which may be useful in the treatment of diseases of abnormal cellular proliferation, e.g. benign prostatic hyperplasia or familial adenomatosis polyposis. The compounds may be useful for the treatment of a variety of cancers as hematopoietic tumors of lymphoid lineage (such as leukemia, acute lymphocytic leukemia, acute lymphoblastic leukemia, B-cell lymphoma, T-cell lymphoma and Hodgkin's lymphoma); hematopoietic tumors of myeloid lineage (such as acute and chronic myelgenous leukemias); carcinomas, tumors of mesenchymal origin; tumors of the central and peripheral nervous system (such as astrocytoma and neuroblastoma) and other tumors such as melanoma and sarcoma.

Another aspect of the present invention therefore relates to a compound according to the first aspect of the present invention for the treatment and/or prophylaxis of a or more disorder and/or disease, selected from the group consisting of glomerulonephritis, uveitis, hepatic diseases or disorders, especially hepatitis, renal diseases or disorders, chronic obstructive pulmonary disease (COPD), rheumatoid arthritis (RA), multiple sclerosis, inflammatory bowel disease (IBD), especially Barrett's oesophagus, ileitis, ulcerative colitis or Crohn's Disease, vasculitis, dermatitis, dermatomyositis, atopic dermatitis, scleroderma, osteoarthritis, inflammatory muscle disease, allergic rhinitis, vaginitis, interstitial cystitis, osteoporosis, eczema, psoriasis, allogeneic or xenogeneic transplantation (cells, stem cells, tissues or organs) graft rejection, graft-versus-host disease, lupus erythematosus, type I diabetes, pulmonary fibrosis, thyroiditis, myasthenia gravis, autoimmune hemolytic anemia, cystic fibrosis, chronic relapsing hepatitis, hepatitis, primary biliary cirrhosis, allergic conjunctivitis, asthma, nasal polyposis; Sjogren's syndrome, cancer and other proliferative diseases, and autoimmune diseases or disorders.

Another embodiment of this aspect of the present invention relates to a compound according to the first aspect of the present invention for the treatment and/or prophylaxis of autoimmune diseases, in particular rheumatoid arthritis and psoriatic arthritis.

Another embodiment of this aspect of the present invention relates to a compound according to the first aspect of the present invention for the treatment and/or prophylaxis of inflammatory disorders of the skin, in particular psoriasis as and/or eczema, most preferably psoriasis.

Another embodiment of this aspect of the present invention relates to a compound according to the first aspect of the present invention for the treatment and/or prophylaxis of chronic inflammatory disorders of the joints, in particular arthritis, rheumatoid arthritis and/or osteoarthritis arthritis, most preferably rheumatoid arthritis (RA).

Yet another embodiment of this aspect of the present invention relates to a compound according to the first aspect of the present invention for the treatment and/or prophylaxis of inflammatory bowel diseases, in particular Barrett's oesophagus, ileitis, ulcerative colitis and Crohn's disease.

Yet another embodiment of this aspect of the present invention relates to a compound according to the first aspect of the present invention for the treatment and/or prophylaxis of allogeneic or xenogeneic transplantation graft rejection, in particular transplantation grafts of cells, stem cells, tissues and/or organs.

Yet another embodiment of this aspect of the present invention relates to a compound according to the first aspect of the present invention for the treatment and/or prophylaxis of autoimmune diseases of the central and peripheral nervous system, in particular multiple sclerosis, myasthenia gravis and/or Eaton-Lambert Myasthenic syndrome, most preferably multiple sclerosis.

Yet another embodiment of this aspect of the present invention relates to a compound according to the first aspect of the present invention for the treatment and/or prophylaxis of inflammatory lung disorders, in particular asthma, acute respiratory distress syndrome, acute lung injury, chronic obstructive pulmonary disease, bronchiectasis and/or cystic fibrosis, most preferably asthma.

Yet another embodiment of this aspect of the present invention relates to a compound according to the first aspect of the present invention for the treatment and/or prophylaxis of allergies, in particular allergic rhinitis.

Another aspect of the present invention provides the use of at least one compound according to the present invention for the preparation of a pharmaceutical composition for the treatment and/or prophylaxis of one or more of the above mentioned diseases and/or disorders.

One embodiment of the invention provides the use of at least one compound according to the present invention for the preparation of a pharmaceutical composition for the treatment and/or prophylaxis of one or more of the diseases and/or disorders, selected from the group consisting of inflammatory disorders and/or autoimmune diseases and/or allergic disorders, preferably selected from the group consisting of psoriasis; psoriatic arthritis; rheumatoid arthritis; inflammatory bowel disease; asthma and allergic rhinitis.

Another aspect of the present invention is a method for the treatment and/or prophylaxis, in particular for of one or more of the above mentioned diseases and/or disorders, in a mammal, in particular in a human, in need of treatment and/or prophylaxis of the respective disease and/or disorder,

which comprises the administration of an effective amount of at least one compound according the present invention or the administration of a pharmaceutical composition according to the invention to the mammal.

One embodiment of the invention is a method for the treatment and/or prophylaxis of disorders and/or diseases, selected from the group consisting of inflammatory disorders and/or autoimmune diseases and/or allergic disorders, preferably selected from the group consisting of psoriasis; psoriatic arthritis; rheumatoid arthritis; inflammatory bowel disease; asthma and allergic rhinitis,

in a mammal, in particular in a human, in need of treatment and/or prophylaxis of the respective disease and/or disorder,
which comprises the administration of an effective amount of at least one compound according the present invention or the administration of a pharmaceutical composition according to the invention to the mammal.

The term “effective amount” according to the present invention means that administered amount of the compound or the pharmaceutical composition that will result in a therapeutically desired biological or medical response of a tissue, system, mammal or human.

A therapeutically desired biological or medical response is understood to be an improved treatment, healing, prevention, or amelioration of a disease, disorder, or side effect, or a decrease in the rate of advancement of a disease or disorder in a mammal, as compared to a corresponding mammal who has not been administered such amount. The term “therapeutically desired biological or medical response” includes also the enhancement of a normal physiological function.

The term “compounds according to the first aspect of the present invention” in foregoing aspects of the invention encompasses all possible stereoisomers and tautomers as well as the respective corresponding acids, bases, salts and solvates.

The embodiments and in particular the preferred embodiments of any aspect of the present invention apply to all other aspects of the inventions respectively.

Compounds of the invention may be made by the methods depicted in the reaction schemes below and described for examples of the invention. The following reaction schemes 1 to 5 are illustrative only and various modifications of the methods may be made by those skilled in the art in order to obtain compounds of the invention. In the following reaction schemes 1 to 5,

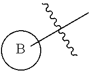

represents the substructure

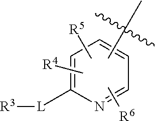

for clarity.

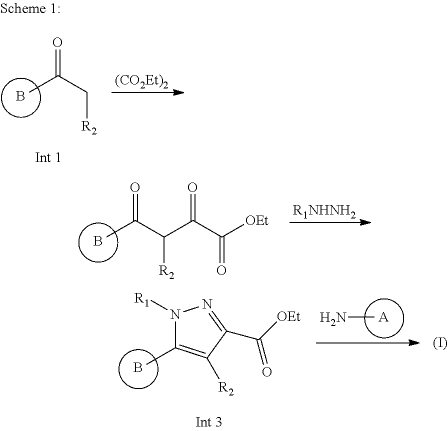

Condensation of an appropriate aryl alkyl ketone with a glyoxalate diester as diethyl glyoxalate yields a β-ketone intermediate that readily cyclises upon treatment with a suitably substituted hydrazine to afford the aryl pyrazole ethyl ester as a mixture of isomers. After separation of the isomers, for instance by flash chromatography, transformation of the ester into compounds of the invention can be performed via saponification and amide coupling by one of the various methods known to those skilled in the art or a conventional one step method (Scheme 1). Alternatively, as shown in Scheme 2 cyclisation of the β-ketone intermediate can be performed with unsubstituted hydrazine. Alkylation with suitable halogenides or equivalents again leads to substituted aryl pyrazole ethyl ester derivatives. Separation of isomers and subsequent steps follow the route depicted in Scheme 1.

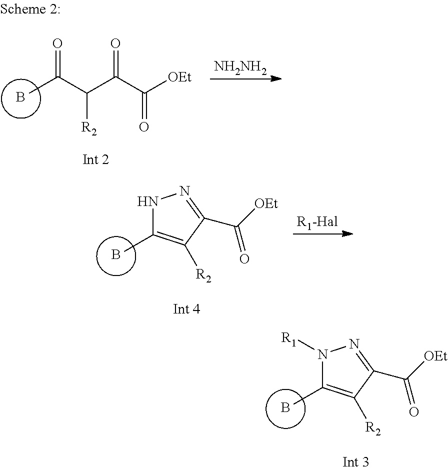

Alternatively, the pyridinyl moiety may be incorporated by direct coupling through a Palladium catalyzed C—H-activation reaction of the 5-unsubstituted pyrazole ester with a pyridinyl halogenide. Subsequent steps may then follow the route depicted in Scheme 1. In particular cases a protecting group may be employed.

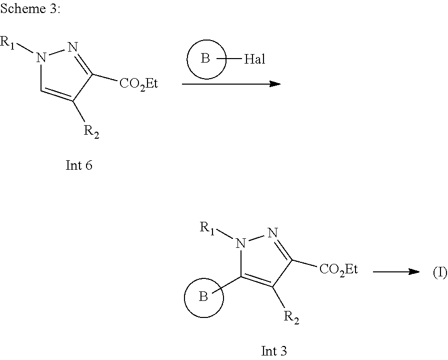

As shown in Scheme 4 and 5 alternatively Pd-catalyzed coupling methods may be used to obtain compounds of the invention. Scheme 4 illustrates the synthesis via a pyrazole bromide or triflate employed in a Suzuki cross coupling with an appropriate boronic acid or ester. The coupling may also be performed on a pyrazole ester intermediate. Scheme 5 provides an example how a 5-unsubstituted pyrazole ester is converted into a boronic ester in the presence of an iridium catalyst and bispinacolatodiborane. Suzuki coupling with an appropriate aryl halogenide or triflate subsequently gives aryl pyrazole esters that can be converted to compounds of the invention as shown in Scheme 1.

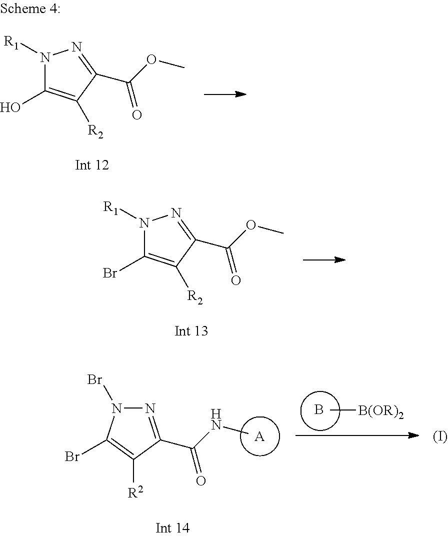

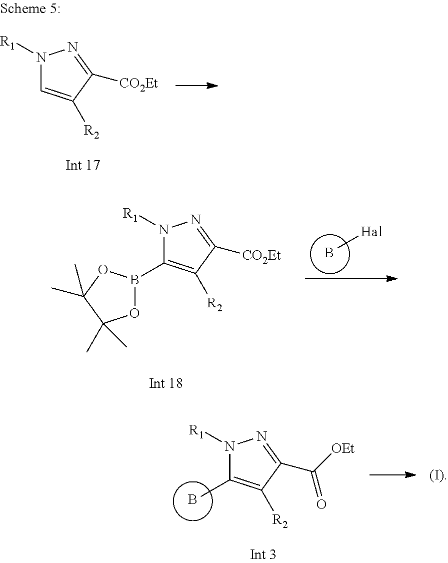

### Exemplified Compounds

The following examples of the invention were prepared according to reaction schemes 1 to 5.

Starting materials and reagents are available from commercial suppliers such as for example Acros, Aldrich, Apollo, Fluke, FluoroChem, Lancaster, Manchester Organics, MatrixScientific, Maybridge, Merck, TCI, Oakwood, etc., or the synthesis has been described as such in the literature or the materials may be prepared by conventional methods known to those skilled in the art.

All the intermediate products and exemplary compounds were analytically characterized by means of ¹H-NMR spectroscopy. In addition, mass spectrometry tests (MS, m/z for [M+H]⁺) were carried out for all the exemplary compounds and selected intermediate products.

### Abbreviations

The indication “equivalents” (“eq.” or “eq” or “equiv.”) means molar equivalents, “RT” or “rt” means room temperature (23±7° C.), “RM” means reaction mixture, “M” are indications of concentration in mol/l, “aq.” means aqueous, “sat.” means saturated, “sol.” means solution, “conc.” means concentrated.

Further abbreviations: Cy=cyclohexane; CC=column chromatography; DIPEA=diisopropylethylamine; DMA=N,N-dimethylacetamide; DMF=N,N-dimethylformamide; EDCl=N-(3-Dimethylaminopropyl)-N′-ethylcarbodiimide hydrochloride; Et₂O=diethyl ether; EtOH=ethanol; EtOAc=ethyl acetate; HATU=1-[bis(dimethylamino)methylene]-1H-1,2,3-triazolo[4,5-b]pyridinium 3-oxid hexafluorophosphate; HOBT=1-hydroxy-benzotriazole; Hex=hexane; iPrOH=iso-propanol; LHMDS=Lithium hexamethyldisilazide; MeOH=methanol; NEt₃=triethyl amine; NMP=N-methyl-pyrrolidinone; NBS=N-bromo-succinimide; Pd(dppf)Cl₂CH₂Cl₂=[1,1′-bis(diphenylphosphino)ferrocene]dichloropalladium(II), complex with CH₂Cl₂; THF=tetrahydrofuran; TFA=trifluoroacetic acid; XPHOS=2-Dicyclohexylphosphino-2′,4′,6′-triisopropylbiphenyl.

### Analytical and Purification Methods:

Liquid Chromatography with Mass Spectrometry Detection: LC-MS

### Method 1:

Agilent LC-MS 1200 Rapid Resolution with detector MSD6140; Detection: MM-ES+APCI+DAD (254 nm): Fragmentation: 50 V [pos/neg]; Column: Agilent SB-C18, 2.1×30 mm, 3.5 micron; Column temperature: 30° C.; Flow rate: 0.8 mL/min; Runtime: 4 min.

Eluent: A: Water; B: MeOH with 1 vol-% formic acid; Gradient: t=0 min: 95/5 (A/B); t=1.00 min: 95/5 (A/B); t=4.00 min: 0/100 (A/B).

### Method 2:

Agilent 1290 Infinity UHPLC-TOF system; Detection: Agilent G4212A DAD (190 400 nm)+Agilent 6224 TOF; Column: Zorbax SB-C18 Rapid Resolution HD, 2.1×50 mm; Column temperature: 80° C.; Flow rate: 2.3 mL/min; Runtime: 1.39 min.

Eluent: A: Water with 0.1 vol-% formic acid; B: CH₃CN with 0.1 vol-% formic acid; Gradient: t=0 min: 98/2 (A/B); t=1.20 min: 0/100 (A/B); t=1.29 min: 0/100 (A/B); t=1.31 min: 98/2 (A/B); t=1.39 min: 98/2 (A/B).

### Method 3:

Applied Biosystem LCMS/MS API 2000; Detection: UV, 220 and 260 nm; Column: Zorbax Extend C18 4.6×50 mm, 5 micron; Column temperature: 30° C.; Flow rate: 1.2 mL/min; Runtime: 5 min.

Eluent: A: Water with 0.05 vol-% formic acid; B: CH₃CN; Gradient: t=0 min: 90/10 (A/B); t=1.50 min: 70/30 (A/B); t=3.00 min: 10/90 (A/B); t=4.00 min: 10/90 (A/B); t=5.00 min: 90/10 (A/B).

### Method 4:

Applied Biosystem LCMS/MS API 2000; Detection: UV, 220 and 260 nm; Column: Zorbax Extend C18 4.6×50 mm, 5 micron; Column temperature: 30° C.; Flow rate: 1.2 mL/min; Runtime: 5 min.

Eluent: A: Water with 10 mM ammonium acetate; B: CH₃CN; Gradient: t=0 min: 90/10 (A/B); t=1.50 min: 70/30 (A/B); t=3.00 min: 10/90 (A/B); t=4.00 min: 10/90 (A/B); t=5.00 min: 90/10 (A/B).

### Method 5:

Applied Biosystem LCMS/MS API 2000; Detection: UV, 220 and 260 nm; Column: Xbrigde C18 4.6×50 mm, 5 micron; Column temperature: 30° C.; Flow rate: 1.2 mL/min; Runtime: 5 min.

Eluent: A: Water with 10 mM ammonium acetate; B: CH₃CN; Gradient: t=0 min: 90/10 (A/B); t=1.50 min: 70/30 (A/B); t=3.00 min: 10/90 (A/B); t=4.00 min: 10/90 (A/B); t=5.00 min: 90/10 (A/B).

### Chromatography

Büchi MPLC system; Stationary phase: silica gel, 40-50μ or

PuriFlash 430; Stationary phase: Interchim®-cartridges.

### NMR Spectroscopy

Bruker Advance II 400 MHz and Bruker Advance II 600 MHz spectrometer.

### Building Block (BB) Synthesis

### BB-1: 5-Bromo-N-(2,6-difluorophenyl)-1-methyl-1H-pyrazole-3-carboxamide

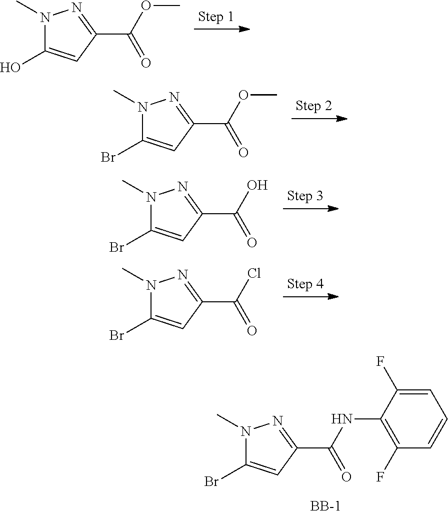

Step 1:

To a solution of methyl 5-hydroxy-1-methyl-1H-pyrazole-3-carboxylate (2.0 g) in CH₃CN (47 mL) was added phosphorus(V) oxybromide (18.3 g) and the mixture was heated to 80° C. for 18 h. The RM was chilled in an ice bath and sat. Na₂CO₃ solution was added. The mixture was extracted with EtOAc, the combined organic layers were dried and the volatiles were removed under reduced pressure to yield the desired product.

LC-MS (Method 2): m/z [M+H]⁺=219.0 (MW calc.=219.04); Rₜ=0.45 min.

Step 2:

A solution of the intermediate of step 1 (506 mg) in dioxane (10 mL) was treated with LiOH solution (2 M, 1 mL) and the mixture was stirred at 70° C. for 1 h. HCl (1 M) was added and the mixture was extracted with EtOAc. The combined organic layers were dried and the volatiles were removed under reduced pressure to yield the desired compound (84%).

LC-MS (Method 2): m/z [M+H]⁺=205.0 (MW calc.=205.01); Rₜ=0.31 min.

Step 3:

A solution of the intermediate of step 2 (2.5 g, 12.2 mmol) in thionyl chloride (21 mL, 293 mmol) was heated to 60° C. for 1 h. The volatiles were removed under reduced pressure to yield the desired compound (2.5 g, 99%).

Step 4:

To a solution of the intermediate of step 3 (800 mg, 3.58 mmol) in CH₂Cl₂ (58 mL) were consecutively added NEt₃ (0.98 mL, 7.16 mmol) and 2,6-difluoroaniline (508 mg, 3.94 mmol) and the resulting mixture was stirred at RT overnight. Sat. NH₄Cl was added, the layers were separated and the aqueous layer was extracted with EtOAc. The combined organic layers were dried and the volatiles were removed under reduced pressure. The residue was purified by chromatography (Interchim® cartridge50SiHP/25 g, Cy/EtOAc) to yield the desired compound (260 mg, 23%).

LC-MS (Method 2): m/z [M+H]⁺=316.0 (MW calc.=316.10); Rₜ=0.77 min.

¹H-NMR (DMSO-d₆): δ=9.91 (s, 1H), 7.44-7.33 (m, 1H), 7.22-7.12 (m, 2H), 6.95 (s, 1H), 3.95 (s, 3H) ppm.

### BB-2: 5-bromo-N-(3-fluoropyridin-4-yl)-1-methyl-1H-pyrazole-3-carboxamide

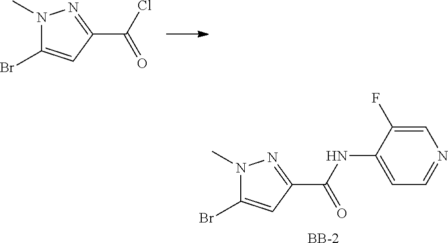

BB-2 was prepared in analogy to the preparation BB-1 through the reaction of the intermediate from step 1 (800 mg, 3.58 mmol) with 3-fluoropyridin-4-amine (440 mg, 3.94 mmol) (849 mg, 79%).

LC-MS (Method 2): m/z [M+H]⁺=299.0 (MW calc.=299.10); Rₜ=0.52 min.

¹H-NMR (DMSO-d₆): δ=9.84 (s, 1H), 8.54 (d, J=4 Hz, 1H), 8.38 (d, J=4 Hz, 1H), 8.04-8.01 (m, 1H), 6.71 (s, 1H), 3.96 (s, 1H) ppm.

### BB-3: 5-bromo-N-(3,5-difluoropyridin-4-yl)-1-methyl-1H-pyrazole-3-carboxamide

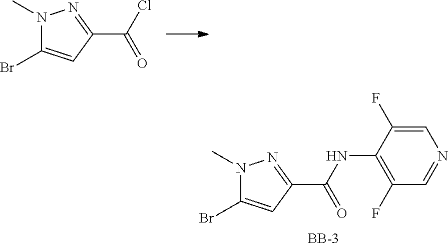

BB-3 was prepared in analogy to the preparation BB-1 through the reaction of the intermediate from step 1 (800 mg, 3.58 mmol) with 3,5-difluoropyridin-4-amine (512 mg, 3.94 mmol) (195 mg, 17%).

LC-MS (Method 2): m/z [M+H]⁺=317.1 (MW calc.=317.09); Rₜ=0.69 min.

¹H-NMR (DMSO-d₆): δ=10.42 (s, 1H), 8.59 (s, 2H), 7.01 (s, 1H), 3.96 (s, 3H) ppm.

### BB-4: Ethyl 1-methyl-5-(4,4,5,5-tetramethyl-1,3,2-dioxaborolan-2-yl)-1H-pyrazole-3-carboxylate

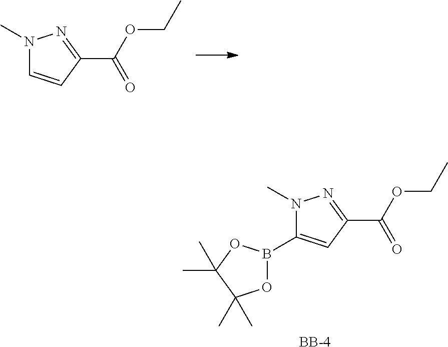

4,4′-Di-tert-butyl-2,2′-dipyridyl (194 mg) was added to a solution of (1,5-Cycloocta-diene)(methoxy)iridium(I) dimer (241 mg) and pinacolborane (4.13 g) in pentane (21 mL) and the mixture was stirred for 20 min at RT. Then a solution of 1-methyl-1H-pyrazole-3-carboxylate (3.05 g) in pentane (14 mL) and THF (7 mL) was added and the solution was stirred at RT for 3 d. The volatiles were removed under reduced pressure and the residue was purified by chromatography (SiO₂, CH₂Cl₂/CH₃OH) to yield the desired product (78%).

¹H-NMR (DMSO-d₆): δ=7.00 (s, 1H), 4.25 (q, J=8 Hz, 2H), 4.04 (s, 3H), 1.31 (s, 3H), 1.28 (t, J=8 Hz, 3H) ppm.

### BB-4a: Methyl 1-methyl-5-(4,4,5,5-tetramethyl-1,3,2-dioxaborolan-2-yl)-1H-pyrazole-3-carboxylate

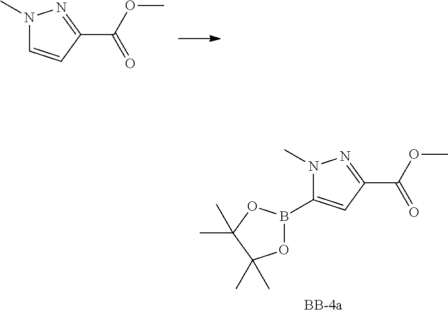

BB-4a was prepared in analogy to the synthesis of BB-4 starting from methyl 1-methyl-1H-pyrazole-3-carboxylate (80%).

### BB-5: (6-Ethoxy-4-methylpyridin-3-yl)boronic acid

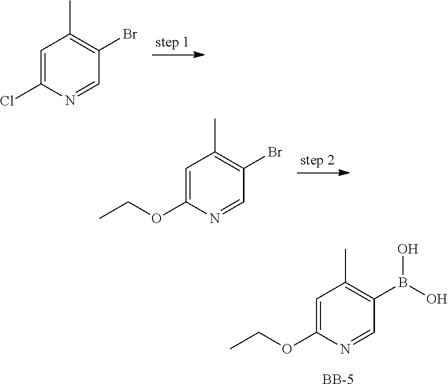

Step 1:

To a solution of 5-bromo-2-chloro-4-methylpyridine (500 mg, 2.42 mmol) in NMP (3.5 mL) was added sodium methoxide (230 mg, 3.39 mmol) and the resulting mixture was heated in a microwave to 150° C. for 30 min. The mixture was diluted with EtOAc and the organic layer was washed with water, was dried and the volatiles were removed under reduced pressure. The residue was purified by chromatography (Interchim® cartridge50SiHP/12 g, Cy/EtOAc) to yield the desired compound (224 mg, 43%).

Step 2:

To a solution of the intermediate of step 1 (99 mg, 463 μmol) in dry THF (1.3 mL) was added at −78° C. n-BuLi (1.6 M in hexane, 0.43 mL) and the mixture was stirred for 2 h. Trimethyl borate (129 μL, 1.16 mmol) was added and the mixture was stirred for another 2 h at −78° C. and the mixture was gradually warmed to RT. Water was added and the volatiles were removed under reduced pressure. The residue was treated with NaOH (1 M) and was washed with Et₂O. The aqueous phase was acidified (pH=3) and was extracted with EtOAc. The volatiles were removed under reduced pressure to yield the desired compound (48 mg, 57%) which was used for the next step without further purification.

### BB-6: 3-bromo-6-ethoxy-2-methylpyridine

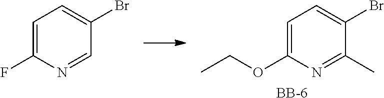

To solution of 3-bromo-6-fluoro-2-methyl-pyridine (500 mg, 2.63 mmol) in EtOH (5 mL) was added a solution of sodium ethoxide in EtOH (5 mL, prepared by addition of 121 mg sodium to 5 ml of EtOH) and the RM was heated to reflux for 1 h. The RM was diluted with water and extracted with EtOAc. The organic layer was dried and the volatiles were removed under reduced pressure to yield the desired compound (560 mg, 98%).

### BB-7: 5-Bromo-2-ethyl-4-methyl pyridine

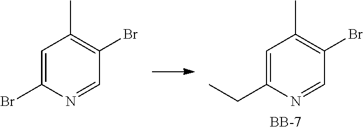

A mixture of 2,5-dibromo-4-methyl-pyridine (4.0 g, 15.9 mmol), diethylzinc (8 mL, 9.56 mmol) and Pd(PPh₃)₄ (184 mg, 159 μmol) in THF (30 mL) was heated under an Ar atmosphere to 70° C. for 1 h. The RM was cooled to RT and was poured into sat. NaHCO₃. The aqueous layer was extracted with Et₂O, the combined organic layers were washed with brine, dried and the volatiles were removed under reduced pressure. The residue was purified by chromatography (SiO₂, EtOAc/Hex) to yield the desired compound (600 mg, 19%).

### BB-8: 5-Bromo-2-cyclopropyl-4-methylpyridine

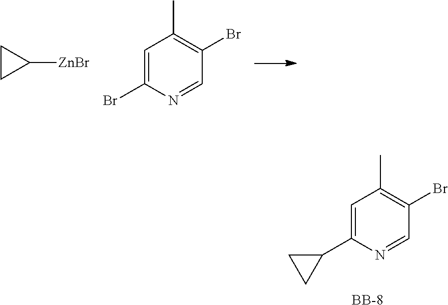

A mixture of 2,5-dibromo-4-methyl-pyridine (500 mg, 1.99 mmol), cyclopropyl zinc bromide (0.5 M in THF, 5.0 mL, 2.49 mmol) and Pd(PPh₃)₄ (40 mg, 20 μmol) in THF (3.2 mL) was heated under an N₂ atmosphere to 70° C. for 1 h. The RM was cooled to RT and was poured into sat. NaHCO₃. The aqueous layer was extracted with Et₂O, the combined organic layers were washed with brine, dried and the volatiles were removed under reduced pressure to yield the desired compound (200 mg, 47%).

LC-MS (Method 2): m/z [M+H]⁺=212.1 (MW calc. 212.09); Rₜ=1.11 min.

### BB-9: 5-Bromo-2-cyclopropoxy-4-methylpyridine

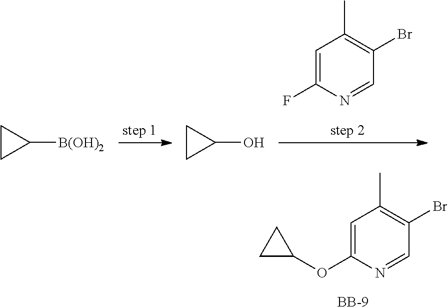

Step 1:

To a suspension of cyclopropylboronic acid (2.5 g, 29.0 mmol) in 10% aqueous NaOH (20 mL) was added drop-wise 30% H₂O₂ (80 mL) at 0° C. and the RM was stirred at same temperature for 1 h. The RM was quenched with Na₂S₂O₃ solution and was extracted with Et₂O. The combined organic layers were dried over CaCl₂ and the mixture was filtered to obtain a solution which was used in next step (Note: this step was done in two parallel batches on 2.5 g scale and the combined solutions were used in the next step).

Step 2:

The solution obtained in step-I (200 mL) was diluted with NMP (50 mL) and was cooled to 0° C. NaH (2.59 g, 64.7 mmol) was added and the mixture was stirred for 20 min. 5-Bromo-2-fluoro-4-methyl-pyridine (2.05 g, 10.77 mmol, 0.55 eq.) was added and the RM was stirred at RT for 16 h. The RM was diluted with cold water and the layers were separated. The organic layer was washed with brine, was dried and the volatiles were removed under reduced pressure. The residue was purified by chromatography (SiO₂CH₂Cl₂/Hex) to yield the desired compound (1.3 g, 19% over 2 steps).

LC-MS (Method 3): m/z [M+H]⁺=228.3 (MW calc. 228.09); Rₜ=3.82 min.

### BB-10: 5-Bromo-4-methyl-2-(trifluoromethoxy)pyridine

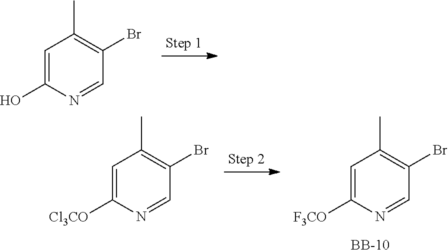

Step 1:

To a suspension of 5-bromo-4-methyl-pyridin-2-ol (5.0 g, 26.6 mmol) in aqueous NaOH (5%, 26 ml) was added drop-wise a solution of thiophosgene (2.4 mL, 31.9 mmol) in CHCl₃ (20 mL) at 0° C. and the RM was stirred for 2 h. The RM was diluted with CHCl₃ and the layers were separated. The aqueous layer was extracted with CHCl₃, the combined organic layers were washed with HCl (1M), water, was dried over sodium sulfate and was filtered. Cl₂ gas was passed through the solution until it reached RT and the mixture was stirred for 2 h. Cl₂ gas was again passed through the RM until a yellow solution was formed and the mixture was stirred at RT for 24 h. The excess Cl₂ gas was removed by purging Ar gas through the mixture. The volatiles were removed under reduced pressure to get the desired compound as crude product which was used without further purification.

Step 2:

The intermediate of step 1 (7.5 g, 24.7 mmol) was added to pre-heated mixture of SbF₃ (8.78 g, 49.3 mmol) and SbCl₅ (468 μl, 3.70 mmol) and the RM was heated to 150° C. for 14 h using a chiller. The RM was chilled and was diluted with CH₂Cl₂ and sat. NaHCO₃ solution to adjust the pH to 8-9. The layers were separated and the organic layer was washed with KF solution (20%), was dried and the volatiles were removed under reduced pressure at 0-5° C. (compound is volatile) to get a crude which was purified by chromatography (SiO₂, Et₂O/Hex) to yield the desired compound (150 mg, 2%).

### BB-11: 5-Bromo-2-isopropoxy-4-methylpyridine

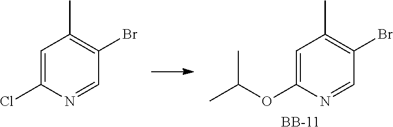

Sodium (43 mg, 1.89 mmol) was dissolved in iPrOH (1.3 mL, 16.8 mmol) and the mixture was heated to 80° C. and was stirred for 30 min. A solution of 5-bromo-2-chloro-4-methylpyridine (300 mg, 1.45 mmol) in iPrOH (1 mL) was added and the mixture was stirred at 80° C. for 30 min. The mixture was chilled, aqueous HCl (1 M) was added and the mixture was extracted with EtOAc. The combined organic layers were washed with brine, were dried and the volatiles were removed under reduced pressure to yield the desired compound (182 mg, 54%) which was used in the next steps without further purification.

¹H-NMR (DMSO-d₆): δ=8.21 (s, 1H), 6.78 (s, 1H), 5.21-5.12 (m, 1H), 2.28 (s, 3H), 1.27 (d, J=6.5 Hz, 6H) ppm.

### BB-12: Ethyl 5-(6-chloro-4-methoxypyridin-3-yl)-1-methyl-1H-pyrazole-3-carboxylate

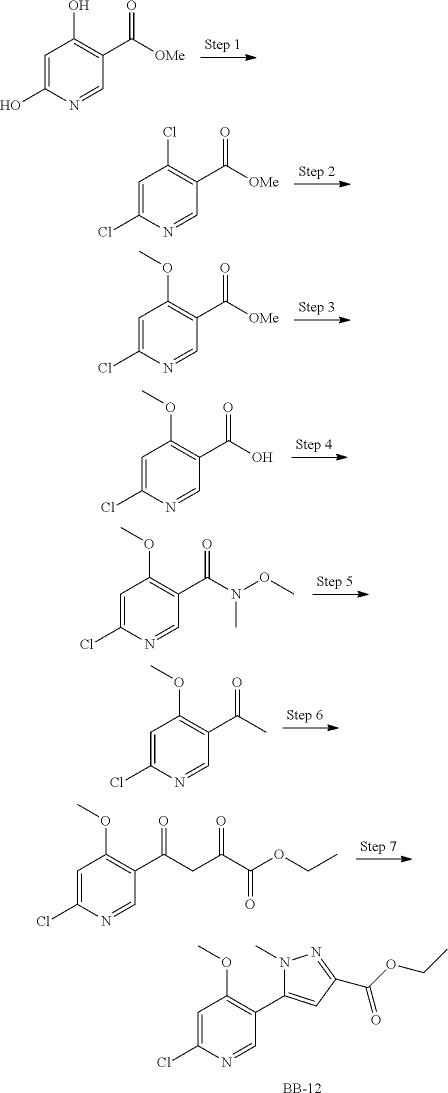

Step 1:

A mixture of 4,6-dihydroxy-nicotinic acid methyl ester (9.10 g, 53.8 mmol) in POCl₃ (46 mL) was heated to reflux for 14 h. The RM was chilled and the volatiles were removed under reduced pressure. The residue was diluted with sat. aqueous Na₂CO₃ solution and was extracted with EtOAc. The combined organic layers were dried and the volatiles were removed under reduced pressure. The residue was purified by chromatography (SiO₂, Hex/EtOAc) to yield the desired compound (4.1 g, 37%).

LC-MS (Method 3): m/z [M+H]⁺=206.1 (MW calc.=206.3); Rₜ=3.46 min.

Step 2:

To a mixture of freshly prepared sodium methoxide (1.05 g, 19.5 mmol) in THF (50 mL) was added drop-wise a solution of the intermediate of step 1 (4.0 g, 19.5 mmol) in THF (30 mL) at 0° C. and the resulting mixture was stirred at RT for 14 h. The volatiles were removed under reduced pressure and the residue was diluted EtOAc. The organic layer was washed with water, was dried and the volatiles were removed under reduced pressure. The residue was purified by chromatography (SiO₂, Hex/EtOAc) to yield the desired compound (2.94 g, 75%).

LC-MS (Method 3): m/z [M+H]⁺=202.3 (MW calc.=201.61); Rₜ=2.95 min.

Step 3:

To a solution of the intermediate of step 2 (2.94 g, 14.6 mmol) in a mixture of MeOH (30 mL), THF (30 mL) and H₂O (3 mL) was added LiOH (1.84 g, 43.9 mmol) at 0° C. and the RM was stirred at RT for 3 h. The volatiles were removed under reduced pressure and the residue was diluted with water and was washed with CH₂Cl₂. The aqueous layer was acidified to pH≈3 using NaHSO₄ solution and was extracted with CH₂Cl₂. The combined organic layers were dried and the volatiles were removed under reduced pressure to yield the desired compound (2.7 g, 98%).

Step 4:

To a solution of the intermediate of step 3 (2.7 g, 14.4 mmol) in CH₂Cl₂ (75 mL) were added DIPEA (7.53 mL, 43.3 mmol) and HATU (5.49 g, 14.4 mmol) at 0° C. and RM was stirred for 15 min. O,N-Dimethyl-hydroxylamine hydrochloride (1.41 g, 14.4 mmol) was added at 0° C. and the RM was stirred at RT for 14 h. The RM was diluted with CH₂Cl₂ and was consecutively washed with water, sat. NaHCO₃, sat. NH₄Cl and brine. The organic layer was dried and the volatiles were removed under reduced pressure. The residue was purified by chromatography (SiO₂, EtOAc/Hex) to yield the desired compound (3.1 g, 93%).

LC-MS (Method 3): m/z [M+H]⁺=231.1 (MW calc.=230.65); Rₜ=2.61 min.

Step 5:

To a solution of the intermediate of step 4 (3.1 g, 13.5 mmol) in THF (70 mL) was added drop-wise methyl magnesium bromide (3M in Et₂O, 6.7 mL, 20.2 mmol) at −10° C. and the RM was stirred for 2 h. Sat. NH₄Cl was added and the mixture was extracted with EtOAc. The combined organic layers were washed brine were dried and the volatiles were removed under reduced pressure. The residue was purified by chromatography (SiO₂, 20% EtOAc/Hex) to yield the desired compound (1.8 g, 72%).

LC-MS (Method 3): m/z [M+H]⁺=186.0 (MW calc.=185.61); Rₜ=2.60 min.

Step 6:

To a mixture of the intermediate of step 5 (3.0 g, 16.2 mmol) and diethyl oxalate (2.2 mL, 16.2 mmol) in THF (80 mL) was added drop-wise LHMDS (1M in THF, 40.5 mL, 40.5 mmol) at RT for 16 h. Sat. NH₄Cl was added and the mixture was extracted with EtOAc. The combined organic layers were dried the volatiles were removed under reduced pressure. The residue was purified by chromatography (SiO₂, EtOAc/Hex) to yield the desired compound (2.65 g, 57%)

LC-MS (Method 3): m/z [M+H]⁺=286.0 (MW calc.=285.68; Rₜ=2.45 min.

Step 7:

To solution of the intermediate of step 6 (5.9 g, 20.7 mmol) in EtOH (150 mL) was added methylhydrazine (1.09 mL, 20.7 mmol) at RT and the RM was heated to reflux for 16 h. The volatiles were removed under reduced pressure and the residue was diluted with EtOAc. The organic layer was washed with water, sat. NH₄Cl and brine, was dried and the volatiles were removed under reduced pressure. The residue was purified by chromatography (SiO₂, EtOAc/Hex) to yield BB-12 (1.24 g, 20%).

LC-MS (Method 3): m/z [M+H]⁺=296.2 (MW calc.=295.72); Rₜ=2.45 min.

### BB-13: 1: 5-Bromo-2-(cyclobutylmethoxy)-4-methylpyridine

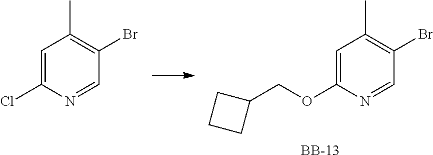

Cyclobutylmethanol (540 mg, 6.30 mmol) was dissolved in dry THF (13 mL) under N₂ atmosphere followed by the addition of sodium (143 mg, 6.30 mmol). The mixture was heated up to reflux for 1.5 h before 5-bromo-2-chloro-4-methylpyridine (1.0 g, 4.84 mmol) was added dropwise and refluxing continued overnight. The mixture was cooled, HCl (1 M) was added and stirring continued for 15 min before extraction with EtOac. The combined organic layers were washed with brine, dried and the volatiles were removed under reduced pressure. The residue was purified by chromatography (SiO₂, EtOAc/Hex) to yield the desired compound (0.6 g, 60%)

LC-MS (Method 2): m/z [M+H]⁺=256.1 (MW calc. 256.14); Rt=1.06 min.

### BB 14: 3-(((5-bromo-4-methylpyridin-2-yl)oxy)methyl)-5-methylisoxazole

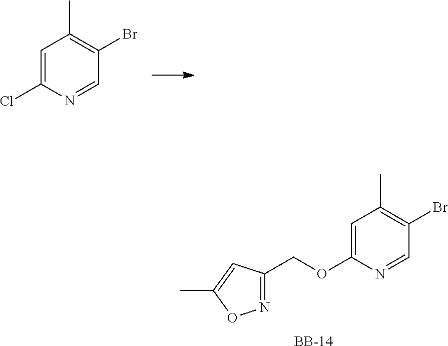

The preparation was performed in analogy to BB-13 employing 3-Hydroxymethyl-5-methylisoxazole (0.18 g, 13%).

### BB-15: 5-bromo-2-(2-methoxyethoxy)-4-methylpyridine

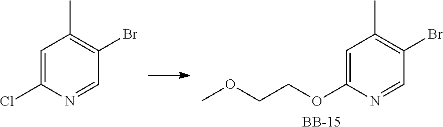

The preparation was performed in analogy to BB-11 employing 2-methoxyethanol (0.96 g, 80%).

LC-MS (Method 2): m/z [M+H]-F=246.1 (MW calc. 246.11); Rt=0.82 min.

### BB-16: 2-((5-bromo-4-methylpyridin-2-yl)oxy)-N,N-dimethylethanamine

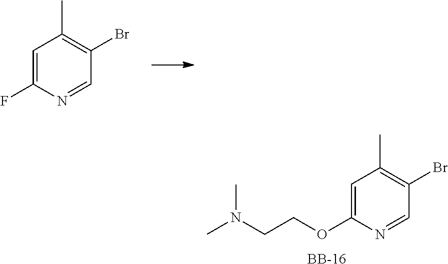

To a solution of 2-dimethylamino-ethanol (0.87 ml, 7.89 mmol) in NMP (3 mL) was added NaH (315 mg, 7.89 mmol, 60% in mineral oil) at 0° C. and the RM was stirred for 30 min. A solution of 5-bromo-2-fluoro-4-methyl-pyridine (500 mg, 2.63 mmol) in NMP (2 mL) was added to the RM and stirred at RT for 16 h. The RM was diluted with ice cold water and extracted with EtOAc. The combined organic layers were washed with water and brine, dried and the volatiles were removed under reduced pressure. The residue was purified by CC (SiO₂, MeOH/CH₂Cl₂) to yield the desired compound (350 mg, 51%).

LC-MS (Method 3): m/z [M]⁺=259.0 (MW calc.=259.14); Rt=2.72 min.

### BB-17: Methyl 5-(6-chloro-4-methylpyridin-3-yl)-1-methyl-1H-pyrazole-3-carboxylate

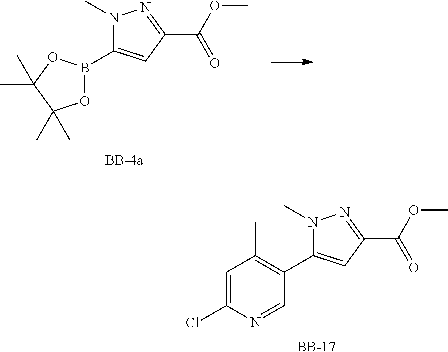

A mixture of BB-4a (780 mg, 2.91 mmol) and 5-bromo-2-chloro-4-methyl-pyridine (600 mg, 2.91 mmol) in DMF (8 mL) was degassed with Ar for 15 min. LiOH (75 mg, 3.12 mmol) and bis-(tri-tert-butylphosphine)palladium (75 mg, 0.15 mmol) was added to the RM and heated at 80° C. for 2 h. The RM was diluted with ice cold water and extracted with EtOAc. The combined organic layers were washed with water, brine and dried over Na₂SO₄. The solvent was evaporated under reduced pressure and the residue purified by CC (SiO₂, EtOAc/Hex) to yield the desired compound (0.40 g, 52%).

LC-MS (Method 3): m/z [M+H]⁺=266.1 (MW calc. 265.70); Rt=2.89 min.

### BB-18: 5-(1-benzyl-4-methyl-6-oxo-1,6-dihydropyridin-3-yl)-N-(2,6-difluorophenyl)-1-methyl-1H-pyrazole-3-carboxamide

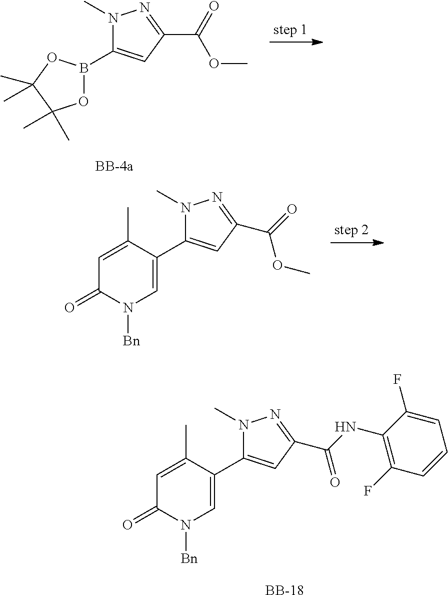

Step 1:

A solution of BB-4a (4.01 g, 15.10 mmol), 1-benzyl-5-bromo-4-methyl-1H-pyridin-2-one (3.0 g, 10.7 mmol) and K₂CO₃ (7.4 g, 53.95 mmol) in a mixture of dioxane (100 mL) and water (20 mL) was degassed with Ar for 30 min. Bis-(tri-tert-butyl phosphine)palladium (275 mg, 0.539 mmol) was added to the RM and heated at 90° C. for 3 h. The RM was concentrated under reduced pressure and diluted with EtOAc before washed with water and brine. The organic layer was dried and concentrated under reduced pressure. The residue was purified by CC (SiO₂, EtOAc/Hex) to give the desired product (3.5 g, 97%).

LC-MS (Method 3): m/z [M+H]⁺=338.0 (MW calc. 337.37); Rₜ=2.81 min.

Step 2:

To a mixture of the intermediate from step 1 (3.3 g, 9.79 mmol) and 2,6-difluoro-phenylamine (1.89 g, 14.68 mmol) in toluene (50 mL) was added drop-wise a 2M solution of Me₃Al in toluene (19.5 mL, 39.16 mmol) and the RM was heated at 80° C. for 14 h. Subsequently it was quenched with sat.NH₄Cl solution and diluted with EtOAc before washed with 1N HCl and brine. The organic layer was dried and concentrated under reduce pressure. The residue was purified by CC (SiO₂, EtOAc/Hex) to give the desired compound (3.5 g, 82%).

LC-MS (Method 3): m/z [M+H]⁺=435.0 (MW calc. 434.44); Rₜ=3.11 min.

### BB-19: 5-(6-(benzyloxy)-4-methylpyridin-3-yl)-1-methyl-1H-pyrazole-3-carbonyl chloride

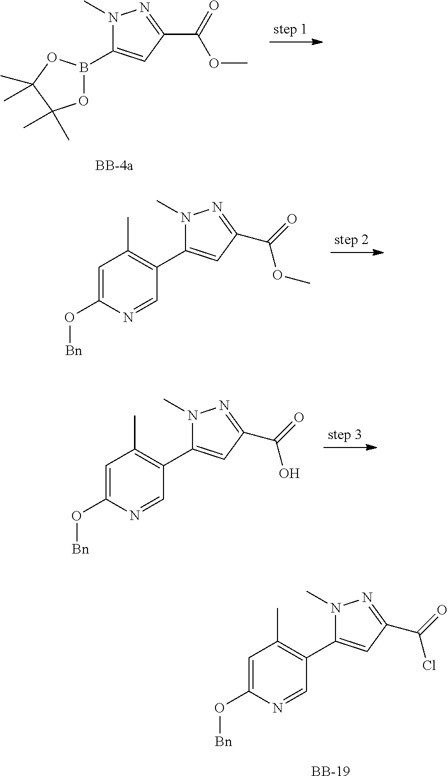

Step 1:

The preparation was performed in analogy to step 1 of BB-18 employing 2-benzyloxy-5-bromo-4-methyl-pyridine (1.0 g, 82%).

LC-MS (Method 3): m/z [M+H]⁺=338.2 (MW calc. 337.37); Rₜ=2.76 min.

Step 2:

To a cooled (0° C.) solution of the intermediate from step 1 (1.0 g, 2.96 mmol) in MeOH:THF (1:1, 4 mL) was added drop-wise a solution of LiOH.H₂O (311 mg, 7.42 mmol) in water (1 mL) and the RM was stirred at RT for 16 h. The RM was concentrated, diluted with water and washed with CH₂Cl₂. The aqueous layer was acidified to pH≈3-4 using NaHSO₄ solution, extracted with CH₂Cl₂ and dried. The solvent was evaporated under reduced pressure to the desired compound (900 mg, 94%).

LC-MS (Method 3): m/z [M−H]⁺=322.0 (MW calc. 323.35); Rₜ=1.62 min

Step 3:

To a cooled (0° C.) suspension of the intermediate from step 2 (390 mg, 1.20 mmol) in CH₂Cl₂ (8 mL) was added oxalyl chloride (0.8 mL) and the RM was stirred at RT for 3 h. The RM was concentrated under N₂ atmosphere to give BB-19 which was used in the next step without further purification.

### BB-20: 5-bromo-2-ethoxy-4-(trifluoromethyl)pyridine

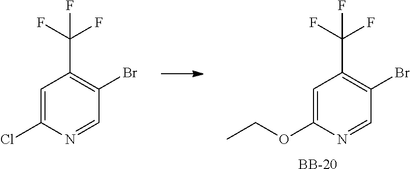

To a solution of 5-bromo-2-chloro-4-trifluoromethyl-pyridine (1.0 g, 3.84 mmol) in EtOH (5 mL) was added freshly prepared 1M solution of sodium ethanolate in EtOH (5.76 mL, 5.76 mmol) at RT and the resulting RM was heated to reflux for 2 h. The RM was concentrated under reduced pressure and subsequently diluted with CH₂Cl₂, washed with water and brine and dried. The solvent was evaporated under reduced pressure to yield the desired compound (700 mg, 67%) which was used in next step without further purification.

### BB-21: 2-chloro-3-fluoro-5-iodo-4-methylpyridine

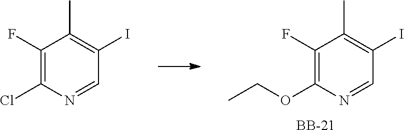

To a solution of 2-chloro-3-fluoro-5-iodo-4-methyl-pyridine (1.5 g, 5.53 mmol) in EtOH (5 mL) was added freshly prepared sodium ethanolate in (1M in EtOH, 8.3 mL, 8.30 mmol) at RT and the resulting mixture was heated to reflux for 4 h. The volatiles were removed under reduced pressure and were diluted with CH₂Cl₂. The organic layer was washed with water and brine, was dried and the volatiles were removed under reduced pressure to yield the desired compound (400 mg, 26%) which was used in next step without further purification.

LC-MS (Method 1): m/z [M+H]⁺=282.1 (MW calc. 281.07); Rₜ=3.99 min.

### BB-22: 5-bromo-N,N,4-trimethylpyridin-2-amine

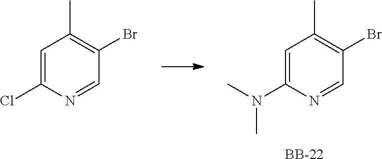

A mixture of 5-bromo-2-chloro-4-methylpyridine (398 mg, 1.93 mmol) and diethyl amine hydrochloride (168 mg, 1.54 mmol) in a mixture of DIPEA (0.5 mL) and DMF (1.4 mL) was heated under Microwave conditions to 200° C. for 12 h. The volatiles were removed under reduced pressure and the residue was purified by chromatography (Interchim® cartridge50SiHP/25 g, Cy/iPrOH) to yield the desired compound (67 mg, 20%).

LC-MS (Method 2): m/z [M+H]⁺=215.1 (MW calc. 215.09); Rₜ=0.37 min.

### BB-23: 5-Bromo-2,4-dimethoxypyridine

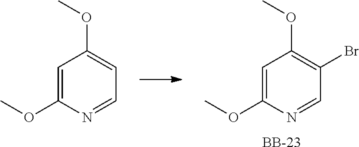

To a solution of 2,4-dimethoxypyridine (800 mg, 5.75 mmol) in CH₃CN (250 mL) was added NBS (1.02 g, 5.75 mmol) and the resulting mixture was heated under exclusion of light to 80° C. for 16 h. The volatiles were removed under reduced pressure and the residue was purified by chromatography (Interchim® cartridge50SiHP/25 g, Cy/EtOAc) to yield the desired compound (444 mg, 35%).

LC-MS (Method 2): m/z [M+H]⁺=218.1 (MW calc. 218.05); Rₜ=0.76 min.

¹H-NMR (DMSO-d₆): δ=8.15 (s, 1H), 6.53 (s, 1H), 3.90 (s, 3H), 3.83 (s, 3H) ppm.

### BB-24: 5-bromo-2-(difluoromethoxy)-4-methoxypyridine

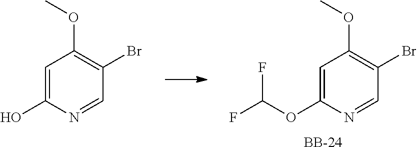

To solution of 5-bromo-4-methoxy-pyridin-2-ol (600 mg, 2.94 mmol) in DMF (5 mL) were added sodium chlorodifluoroacetate (535 mg, 3.52 mmol) and Cs₂CO₃ (1.43 g, 4.41 mmol) and the RM was heated at 100° C. for 2 h. The RM was treated with ice cold water and was extracted with diethyl ether. The combinded organic layers were washed with water, brine were dried and the volatiles were removed under reduced pressure to get crude compound which was purified by CC (SiO₂, EtOAc/Hex) to yield the desired compound (300 mg, 1.18 mmol) (300 mg, 40%).

### BB-25: 5-bromo-2-chloro-4-(difluoromethoxy)pyridine

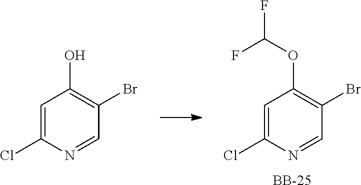

To solution of 5-bromo-2-chloro-pyridin-4-ol (800 mg, 3.84 mmol) in DMF (10 ml) were added sodium chlorodifluoroacetate (700 mg, 4.61 mmol) and Cs₂CO₃ (1.87 g, 5.76 mmol) and the RM was heated at 100° C. for 2 h. The RM was treated with ice cold water and was extracted with diethyl ether. The combined organic layers were washed with water, brine, were dried and the volatiles were removed under reduced pressure to get a crude compound which was purified by CC (SiO₂, EtOAc/Hex) to afford the desired compound (600 mg, 60%).

### BB-26: 5-bromo-2-cyclopropyl-4-methoxypyridine

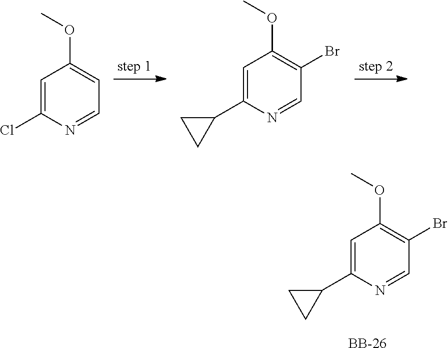

Step 1:

A mixture of 2-chloro-4-methoxypyridine (700 mg, 4.88 mmol), cyclopropyl zinc bromide (0.5M in THF, 12.2 mL, 6.09 mmol) and Pd(PPh₃)₄ (96 mg, 49 μmol) in THF (7.9 mL) was stirred under N₂ at 70° C. overnight. Aqueous NaHCO₃ was added and the mixture was extracted with EtOAc. The combined organic layers were dried and the volatiles were removed under reduced pressure. The residue was purified through chromatography (Interchim® cartridge50SiHP/12 g, Cy/EtOAc) to yield the desired compound (600 mg, 83%).

Step 2:

A solution of step 1 intermediate (2.00 g, 13.4 mmol) in CH₃CN (495 mL) was treated with NBS (2.39 g, 13.4 mmol) and the mixture was stirred in the dark for at 80° C. overnight. The volatiles were removed under reduced pressure and the residue was purified by chromatography (Interchim® cartridge50SiHP/12 g, Cy/EtOAc) to yield the desired compound (917 mg, 30%).

¹H-NMR (DMSO-d₆): δ=8.33 (s, 1H), 7.09 (s, 1H) 3.93 (s, 3H) 2.12.-2.04 (m, 1H), 0.98-0.88 (m, 4H) ppm.

### BB-27: 5-bromo-2-methoxy-4-methylpyridine

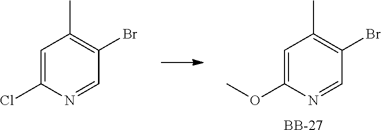

Sodium (143 mg, 6.30 mmol) was dissolved in methanol (1.8 mL) at 80° C. and 5-bromo-2-Chloro-4-methylpyridine (1.00 g, 4.84 mmol) in methanol (1 mL) was added. The mixture was stirred at 80° C. overnight and 1M HCl was added. The mixture was extracted with EtOAc, the combined organic layers were dried and the volatiles were removed under reduced pressure to yield the desired compound (770 mg, 79%).

¹H-NMR (DMSO-d₆): δ=8.24 (s, 1H), 6.87 (s, 1H), 3.82 (s, 3H), 2.30 (s, 3H) ppm.

### BB-28: 5-bromo-2-(methoxymethyl)-4-methylpyridine

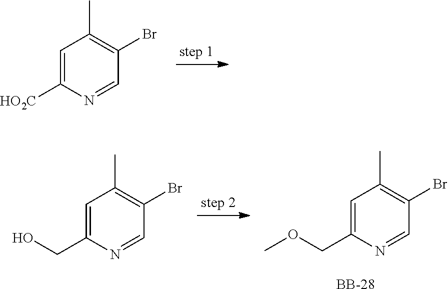

Step 1:

To a solution of 5-bromo-4-methylpicolinic acid (400 mg, 1.85 mmol) in dry THF (3 mL) was added borane THF complex (1M in THF, 7.4 mL, 7.4 mmol) at 0° C. and the mixture was stirred at RT overnight. The mixture was cooled to 0° C. and aqueous NH₄Cl was added. The mixture was extracted with EtOAc, the combined organic layers were dried and the volatiles were removed under reduced pressure (308 mg, 82%).

Step 2:

Sodium hydride (60% in mineral oil, 11 mg, 779 μmol) was added to a solution of step 1 intermediate (150 mg, 742 μmol) in THF (2.7 mL) at 0° C. and the mixture was stirred for 1 h. Methyl iodide (105 μL, 742 μmol) was added and the mixture was stirred at RT for 3 d. Aqueous NH₄Cl was added and the mixture was extracted with EtOAc. The combined organic layers were dried and the volatiles were removed under reduced pressure. The residue was purified by chromatography (SiO₂, 12 g, Cy/EtOAc) to yield the desired compound (144 mg, 90%).

¹H-NMR (DMSO-d₆): δ=8.58 (s, 1H), 7.41 (s, 1H), 4.43 (s, 2H), 3.36 (s, 3H), 2.37 (s, 3H) ppm.

### BB-29: 4-methyl-5-(4,4,5,5-tetramethyl-1,3,2-dioxaborolan-2-yl)-2-(trifluoromethyl)pyridine

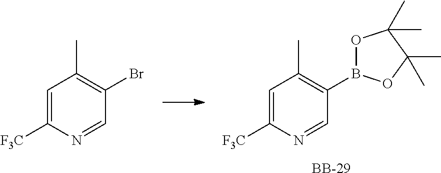

A mixture of 5-bromo-4-methyl-2-(trifluoromethyl)pyridine (150 mg, 625 μmol), bis(pinacolato)diboron (189 mg, 729 μmol), Pd(dppf)Cl₂ (89 mg, 106 μmol) and potassium acetate (120 mg, 1.25 mmol) in dioxane (1 mL) was stirred under N₂ at 90° C. overnight. Aqueous NH₄Cl was added and the mixture was extracted with CH₂Cl₂. The combined organic layers were dried and the volatiles were removed under reduced pressure to yield the desired compound which was used in the next step without further purification.

### BB-30: 5-bromo-2-ethoxy-4-(trifluoromethyl)pyridine

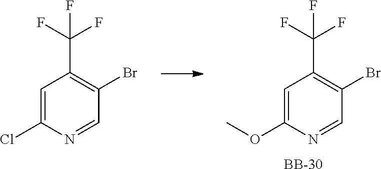

To a solution of 5-bromo-2-chloro-4-trifluoromethyl-pyridine (2.54 g, 9.80 mmol) in MeOH (25 mL) was added 25% NaOMe in MeOH (3.2 ml, 14.70 mmol) and heated at reflux for 2 h. The RM was diluted with water and extracted with hexane. The combined organic layers were washed with water and brine, dried and concentrated under reduced pressure to give 5-bromo-2-methoxy-4-trifluoromethyl-pyridine(1.7 g, 68%).

### BB-31: 5-bromo-2-cyclopropoxy-4-methoxypyridine

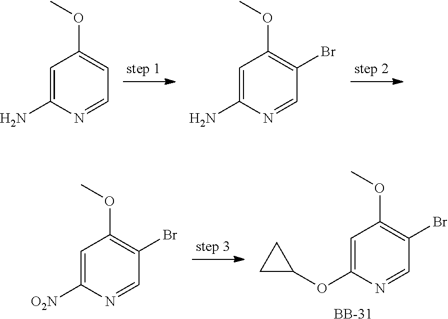

Step 1:

To a solution of 4-methoxy-pyridin-2-ylamine (10.0 g, 80.64 mmol) in acetic acid (320 mL) was added Br₂ (3.75 mL, 72.58 mmol) in acetic acid (80 mL) dropwise at RT and the resulting RM was stirred at RT for 1 h. The solid precipitate was separated by filtration, washed with hexane and dried to yield 5-bromo-4-methoxy-pyridin-2-ylamine (13.0 g, 79%).

LC-MS (Method 4): m/z [M+1]⁺=203.1 & 204.8 (MW calc. 203.04); Rₜ=2.43 min.

Step 2:

To a mixture of H₂O₂ (16 mL) and conc. H₂SO₄ (20 mL) was added dropwise a solution of 5-bromo-4-methoxy-pyridin-2-ylamine (2.0 g, 9.85 mmol) in conc. H₂SO₄ (20 mL) at 0° C. and the resulting RM was stirred at RT for 3 h. The RM was neutralized with sat.NaHCO₃ solution up to pH˜7-8 and extracted with EtOAc. The organic layer was dried and concentrated under reduced pressure to give the crude product which was purified by CC (SiO₂; 4% EtOAc/Hex) to yield 5-bromo-4-methoxy-2-nitro-pyridine (470 mg, 20%).

LC-MS (Method 4): m/z [M+1]⁺=232.9 and 234.9 (MW calc. 233.02); Rₜ=2.96 min.

Step 3:

A crude cyclopropanol solution in Et₂O (50 mL, synthesized from 3.13 g of cyclopropyl boronic acid and H₂O₂) was diluted with NMP (15 mL) and cooled to 0° C. NaH (60% in mineral oil, 721 mg, 18.02 mmol) was added at 0° C. and the RM was stirred at same temperature for 10 min. A solution of 5-bromo-4-methoxy-2-nitro-pyridine (700 mg, 3.0 mmol) in NMP (15 mL) was added dropwise and the RM was stirred at RT for 30 min. The RM was quenched with cold water and extracted with 20% EtOAc/Hex. The organic layer was dried and concentrated under reduced pressure to give the crude product which was purified by CC (SiO₂; 2% EtOAc/Hex) to yield 5-bromo-2-cyclopropoxy-4-methoxy-pyridine (BB-31, 300 mg, 40%).

LC-MS (Method 4): m/z [M+1]⁺=244.0 and 246.0 (MW calc. 244.09); Rₜ=3.37 min.

### BB-32: 5-bromo-2-ethoxy-4-methoxypyridine

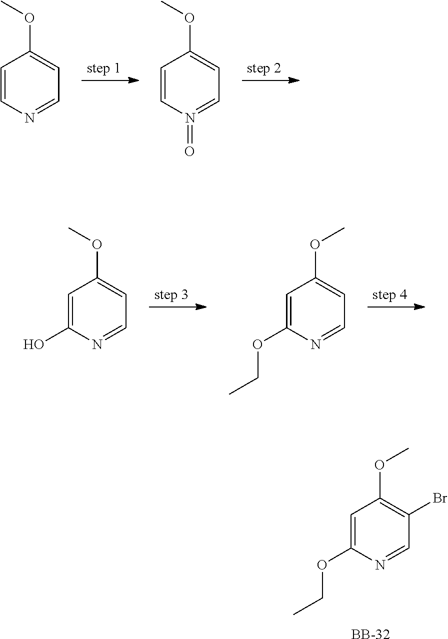

Step 1:

To a stirred solution of 4-methoxy pyridine (10.0 g, 9.17 mmol) in acetic acid (50 mL) was added H₂O₂ (30%, 25 mL) and the RM was heated to reflux for 24 h. Then the RM was cooled to RT and evaporated to afford 4-Methoxy-pyridine 1-oxide (9.0 g). This crude material was used in the next step with out any purification.

Step 2:

A stirred solution of step 1 intermediate (30.0 g, 240 mmol) in acetic acid anhydride (900 mL) was refluxed for 16 h. The volatiles were removed under reduced pressure and the residue was dissolved in methanol (300 mL) and water (300 mL) and the mixture was stirred for 16 h at RT. The volatiles were removed under reduced pressure and the crude material was purified by CC (SiO₂, CH₂Cl₂/MeOH) to the desired compound (20 g, 66%).

Step 3:

To a stirred solution of step 2 intermediate (15.0 g, 120 mmol) in CHCl₃ (500 mL) was added Ag₂CO₃ (49.5 g, 180 mmol) followed by ethyl iodide (28.8 mL, 360 mmol) and the RM was stirred for 16 h at RT. After completion the RM was filtered and washed with CH₂Cl₂. The filtrate was washed with saturated NaHCO₃ and brine, dried and the volatiled were removed under reduced pressure. The residue was purified by CC (SiO₂, EtOAc/Hex) to afford the desired compound (12.0 g, 65%).

Step 4:

To a stirred solution of step 3 intermediate (20 g, 131 mmol) in CH₃CN (6 L), was added NBS (23.2 g, 131 mmol) slowly and the RM was refluxed for 16 h. The volatiles were removed under reduced pressure and the crude was purified by CC (SiO₂, EtOAc/Hex) to BB32) (16 g, 53%).

¹H-NMR (400 MHz, DMSO-d₆): δ=8.13 (s, 1H), 6.51 (s, 1H), 4.30 (q, J=7.0 Hz, 2H), 3.89 (s, 3H), 1.31 (t, J=7.0 Hz, 2H) ppm.

### SYNTHESIS OF THE REPRESENTATIVE EXAMPLES

### Example 1

### 5-(6-Ethoxy-4-methyl-pyridin-3-yl)-N-(3-fluoro-pyridin-4-yl)-1-methyl-1H-pyrazole-3-carboxylic acid amide

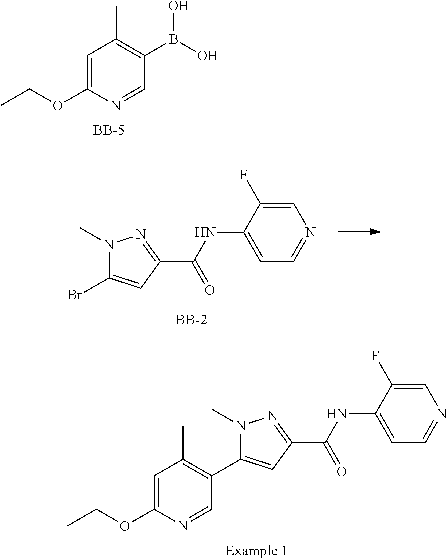

A mixture of BB-4 (36 mg, 201 μmol), BB-2 (39 mg, 134 μmol) and Pd(dppf)Cl₂.CH₂Cl₂ (7 mg, 7 μmol) in a mixture of THF (0.8 mL) and aqueous Na₂CO₃ (2M, 0.2 mL) was heated in a microwave under N₂ atmosphere to 100° C. for 3 h. The mixture was chilled, saturate NH₄Cl was added and the mixture was extracted with EtOAc. The organic layer was dried and the volatiles were removed under reduced pressure. The residue was purified by chromatography (Interchim® cartridge50SiHP/12 g, Cy/EtOAc) to yield the desired compound (20 mg, 42%).

LC-MS (Method 2): m/z [M+H]⁺=356.1 (MW calc.=355.37); Rₜ=0.68 min.

¹H-NMR (DMSO-d₆): δ=9.80 (s, 1H), 8.59 (d, J=1.5 Hz, 1H), 8.39 (d, J=5.3 Hz, 1H), 8.13-8.07 (m, 2H), 6.93 (s, 1H), 6.85 (s, 1H), 4.36 (q, J=6.8 Hz, 2H), 3.76 (s, 3H), 2.14 (s, 3H), 1.34 (t, J=7.2 Hz, 3H) ppm.

### Example 2

### N-(3,5-Difluoro-pyridin-4-yl)-5-(6-ethoxy-4-methyl-pyridin-3-yl)-1-methyl-1H-pyrazole-3-carboxylic acid amide

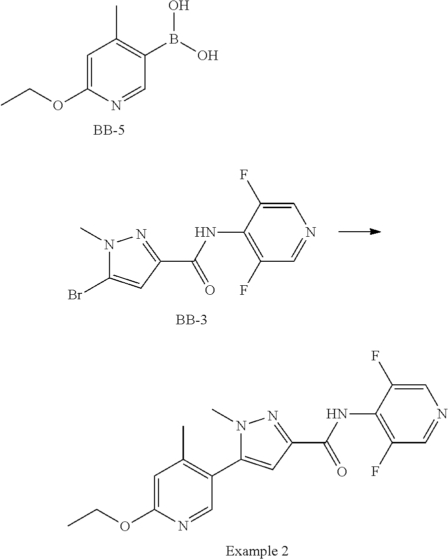

The title compound synthesized in analogy to example 1 through the reaction of BB-4 (42 mg, 237 μmol) with BB-3 (50 mg, 158 μmol) (42 mg, 71%).

LC-MS (Method 2): m/z [M+H]⁺=374.1 (MW calc.=373.36); Rₜ=0.70 min.

¹H-NMR (DMSO-d₆): δ=10.37 (s, 1H), 8.59 (s, 2H), 8.10 (s, 1H), 6.88 (s, 1H), 6.85 (s, 1H), 4.36 (q, J=7.5 Hz, 2H), 3.75 (s, 3H), 2.15 (s, 3H), 1.34 (t, J=7.2 Hz, 2H) ppm.

### Example 3

### N-(2,6-Difluoro-phenyl)-5-(6-ethoxy-4-methyl-pyridin-3-yl)-1-methyl-1H-pyrazole-3-carboxylic acid amide

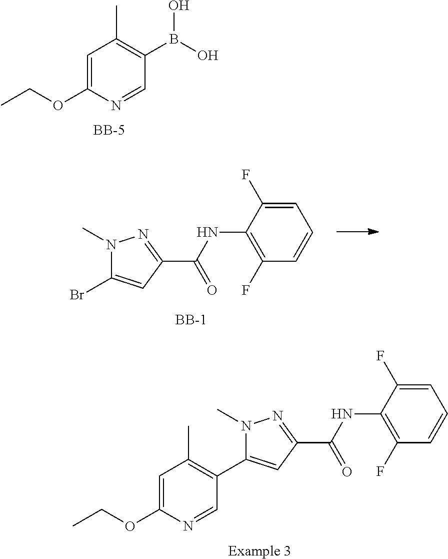

The title compound was synthesized in analogy to example 1 through the reaction of BB-4 (43 mg, 237 μmol) with BB-1 (50 mg, 158 μmol) (25 mg, 42%).

LC-MS (Method 2): m/z [M+H]⁺=373.2 (MW calc.=372.38); Rₜ=0.76 min.

¹H-NMR (DMSO-d₆): δ=9.87 (s, 1H), 8.10 (s, 1H), 7.42-7.36 (m, 1H), 7.20-7.15 (m, 2H), 6.84 (s, 1H), 6.82 (s, 1H), 4.35 (q, J=6.8 Hz, 2H), 3.73 (s, 3H), 2.15 (s, 3H), 1.34 (t, J=7.2 Hz, 3H) ppm.

### Example 4

### N-(3,5-Difluoro-pyridin-4-yl)-5-(6-ethoxy-2-methyl-pyridin-3-yl)-1-methyl-1H-pyrazole-3-carboxylic acid amide

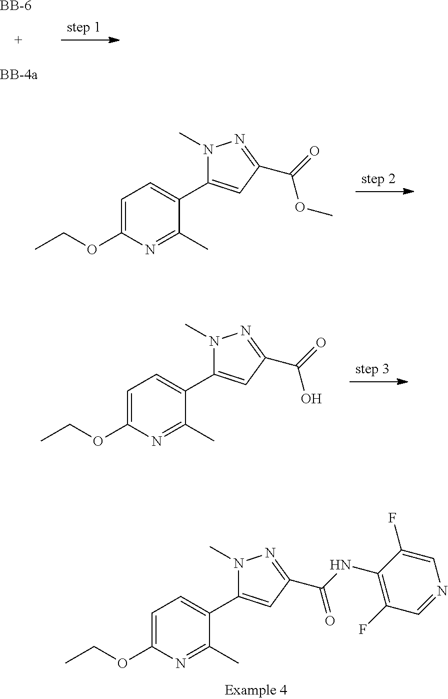

Step 1:

A solution of intermediate 4a (500 mg, 1.88 mmol), intermediate BB-6 (485 mg, 2.25 mmol) and K₂CO₃ (1.3 g, 9.42 mmol) in dioxane (5 mL) and water (1 mL) was degassed with Ar for 30 min. Bis-(tri-tert-butyl phosphine)palladium (48 mg, 90.0 μmol) was added to and the RM was heated to 80° C. for 4 h. The volatiles were removed under reduced pressure and the residue was diluted with EtOAc and was washed with water and brine. The organic layer was dried and the volatiles were removed under reduced pressure. The residue was purified by chromatography (SiO₂, EtOAc/Hex) to yield the desired compound (420 mg, 81%).

Step 2:

To a solution of the intermediate of step 1 (420 mg, 1.53 mmol) in a mixture of MeOH (4 mL) and THF (4 mL) was added drop-wise a solution of LiOH (156 mg, 3.82 mmol) in water (2 mL) and the RM was stirred at RT for 4 h. The volatiles were removed under reduced pressure and the residue was diluted with water and was washed with CH₂Cl₂. The aqueous layer was acidified to pH≈3 using NaHSO₄ solution and the formed solid was isolated by filtration to yield the desired compound (350 mg, 87%).

Step 3:

To a suspension of the intermediate of step 2 (100 mg, 0.38 mmol) in CH₂Cl₂ (3 mL) was added at 0° C. a catalytic amount of DMF (2 drops) followed by oxalyl chloride (82 μl, 0.95 mmol) and the RM was stirred at RT for 2 h. The volatiles were removed under reduced pressure and the residue was dissolve in CH₂Cl₂ (5 mL). To this solution were consecutively added DIPEA (0.2 mL, 1.14 mmol) and 3,5-difluoro-pyridin-4-ylamine (60 mg, 0.45 mmol) at 0° C. and the RM was stirred at RT for 14 h. The RM was diluted with CH₂Cl₂ and was washed with water, sat. NH₄Cl and brine (40 ml). The organic layer was dried and the volatiles were removed under reduced pressure. The residue was purified by chromatography (SiO₂, EtOAc/Hex) to yield the desired compound (53 mg, 37%).

LC-MS (Method 2): m/z [M+H]⁺=374.1 (MW calc.=373.36); Rₜ=0.70 min.

¹H-NMR (DMSO-d₆): δ=10.40 (s, 1H), 8.59 (s, 2H), 7.67 (d, J=8.4 Hz, 1H), 6.86 (s, 1H), 6.76 (d, J=8.4 Hz, 1H), 4.35 (q, J=7.0 Hz, 2H), 3.73 (s, 3H), 2.28 (s, 3H), 1.34 (t, J=7.04 Hz, 3H) ppm.

### Example 5

### N-(2,6-Difluoro-phenyl)-5-(6-ethoxy-2-methyl-pyridin-3-yl)-1-methyl-1H-pyrazole-3-carboxylic acid amide

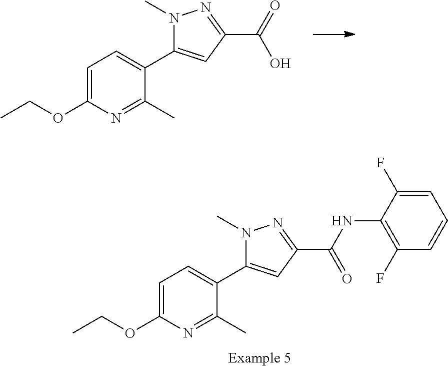

The title compound was prepared in analogy to example 4 through the reaction of intermediate of step 2 (150 mg, 0.57 mmol) with 2,6-difluorophenylamine (90 mg, 0.67 mmol) (65 mg, 30%).

LC-MS (Method 2): m/z [M+H]⁺=373.1 (MW calc.=372.37); Rₜ=0.76 min.

¹H-NMR (DMSO-d₆): δ=9.90 (s, 1H), 7.66 (d, J=8.4 Hz, 1H), 7.40-7.37 (m, 1H), 7.18 (t, J=8.0 Hz, 2H), 6.80 (s, 1H), 6.76 (d, J=8.2 Hz, 1H), 4.36 (q, J=7.1 Hz, 2H), 3.72 (s, 3H), 2.29 (s, 3H), 1.34 (t, J=7.1 Hz, 3H) ppm.

### Example 6

### N-(2,6-Difluoro-phenyl)-5-(6-ethyl-4-methyl-pyridin-3-yl)-1-methyl-1H-pyrazole-3-carboxylic acid amide

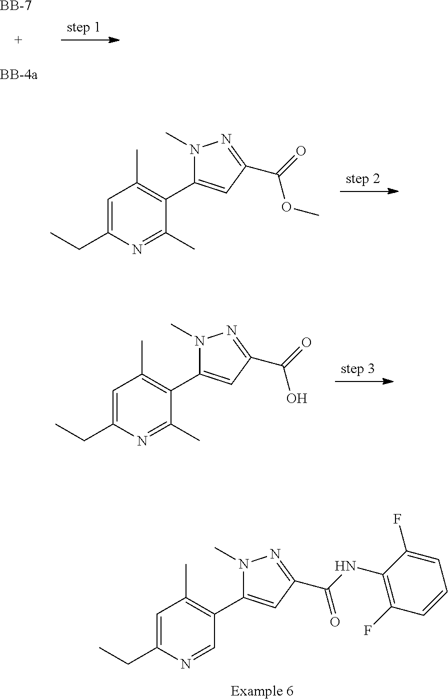

The title compound was prepared in analogy to example 4 starting from BB-7.

LC-MS (Method 2): m/z [M+H]⁺=357.2 (MW calc.=356.38); Rₜ=0.50 min.

¹H-NMR (DMSO-d₆): δ=9.93 (s, 1H), 8.40 (s, 1H), 7.41-7.37 (m, 1H), 7.32 (s, 1H), 7.18 (t, J=8.0 Hz, 2H), 6.86 (s, 1H), 3.74 (s, 3H), 2.78 (q, J=7.6 Hz, 2H), 2.19 (s, 3H), 1.27 (t, J=7.52 Hz, 3H) ppm.

### Example 7

### N-(3,5-Difluoro-pyridin-4-yl)-5-(6-ethyl-4-methyl-pyridin-3-yl)-1-methyl-1H-pyrazole-3-carboxylic acid amide

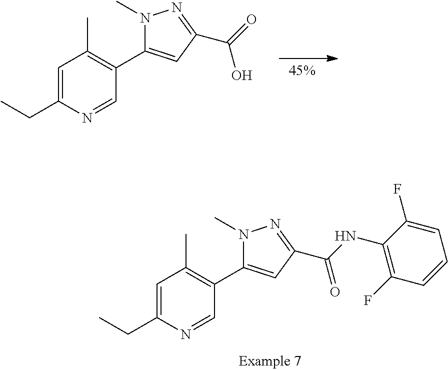 [SMILES: CCc1cc(C)c(-c2cc(C(=O)O)nn2C)cn1 ; CCc1cc(C)c(-c2cc(C(=O)Nc3c(F)cccc3F)nn2C)cn1]

The title compound was prepared in analogy to example 4 starting from step 2 intermediate of example 6.

LC-MS (Method 2): m/z [M+H]⁺=358.1 (MW calc.=357.36); Rₜ=0.43 min.

¹H-NMR (DMSO-d₆): δ=10.43 (s, 1H), 8.60 (s, 2H), 8.40 (s, 1H), 7.32 (s, 1H), 6.91 (s, 1H), 3.75 (s, 3H), 2.78 (q, J=7.6 Hz, 2H), 2.19 (s, 3H), 1.27 (t, J=7.6 Hz, 3H) ppm.

### Example 8

### 5-(6-Cyclopropyl-4-methyl-pyridin-3-yl)-N-(2,6-difluoro-phenyl)-1-methyl-1H-pyrazole-3-carboxylic acid amide

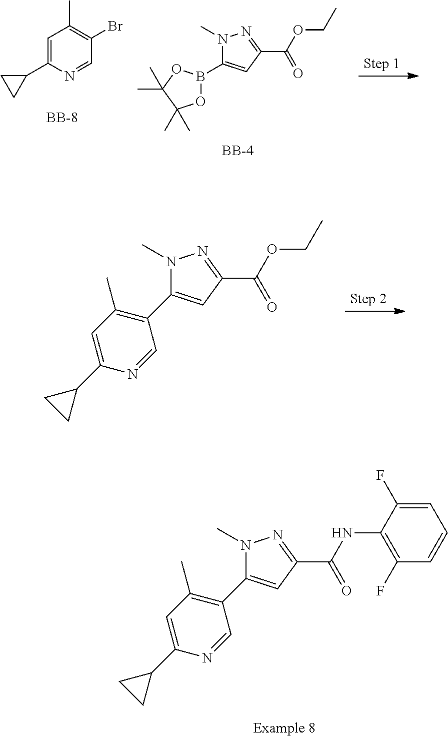

The title compound was prepared in analogy to example 4 starting from BB-8.

LC-MS (Method 2): m/z [M+H]⁺=369.2 (MW calc.=368.38); Rₜ=0.8 min.

¹H-NMR (DMSO-d₆): δ=9.88 (s, 1H), 8.31 (s, 1H), 7.42-7.36 (m, 1H), 7.34 (s, 1H), 7.21-7.15 (m, 2H), 6.83 (s, 1H), 3.30 (s, 3H), 3.13 (s, 3H), 2.15-2.10 (m, 1H), 1.02-0.95 (m, 4H) ppm.

### Example 9

### 5-(6-Cyclopropyloxy-4-methyl-pyridin-3-yl)-N-(3,5-difluoro-pyridin-4-yl)-1-methyl-1H-pyrazole-3-carboxylic acid amide

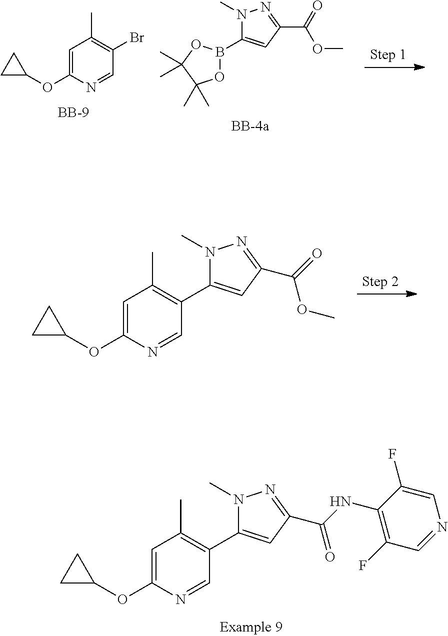

Step 1:

A mixture of BB-4a (2.13 g, 7.98 mmol), BB-9 (1.30 g, 5.70 mmol) and K₂CO₃ (3.93 g, 28.5 mmol) in dioxane (52 mL) and water (11 mL) was degassed through purging with Ar for 30 min. Bis(tri-tert-butylphosphine)palladium (146 mg, 0.28 mmol) was added and the RM was heated to 80° C. for 16 h. The RM was cooled, diluted with water and was extracted with EtOAc. The combined organic layers were washed with brine, dried and the volatiles were removed under reduced pressure. The residue was purified by chromatography (SiO₂, CH₂Cl₂/MeOH) to yield the desired compound (1.5 g, 91%)

LC-MS (Method 3): m/z [M+H]⁺=288.0 (MW calc. 287.31); Rₜ=2.97 min.

Step 2:

To a solution of 3,5-difluoro-pyridin-4-yl amine (340 mg, 2.61 mmol) in dry toluene (15 mL) was added Me₃Al (2M in toluene, 2.6 mL, 5.23 mmol) at 0° C. and the mixture was stirred for 30 min. A solution of the step 1 intermediate (500 mg, 1.74 mmol) in toluene (5 mL) was added and the RM was heated to reflux for 16 h. The RM was cooled and sat. NH₄Cl solution (25 mL) was added and the mixture was extracted with EtOAc. The combined organic layers were washed with sat. NaHCO₃, water and brine, were dried and the volatiles were removed under reduced pressure. The residue was purified by chromatography (SiO₂, CH₂Cl₂/MeOH) to the desired compound (160 mg, 24%).

LC-MS (Method 2): m/z [M+H]⁺=386.1 (MW calc.=385.37); Rₜ=0.65 min.

¹H-NMR (DMSO-d₆): δ=10.40 (s, 1H), 8.59 (s, 2H), 8.15 (s, 1H), 6.92 (s, 1H), 6.89 (s, 1H), 4.28-4.22 (m, 1H), 3.76 (s, 3H), 2.16 (s, 3H), 0.80-0.70 (m, 4H) ppm.

### Example 10

### 5-(6-Cyclopropyloxy-4-methyl-pyridin-3-yl)-N-(2,6-difluoro-phenyl)-1-methyl-1H-pyrazole-3-carboxylic acid amide

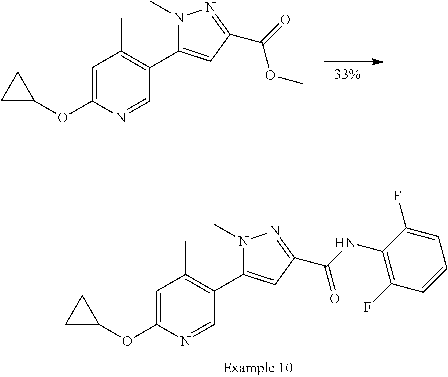 [SMILES: COC(=O)c1cc(-c2cnc(OC3CC3)cc2C)n(C)n1 ; Cc1cc(OC2CC2)ncc1-c1cc(C(=O)Nc2c(F)cccc2F)nn1C]

The title compound was prepared in analogy to example 9 starting from step 1 intermediate of example 9.

LC-MS (Method 2): m/z [M+H]⁺=385.1 (MW calc.=384.38); Rₜ=0.72 min.

¹H-NMR (DMSO-d₆): δ=9.91 (s, 1H), 8.15 (s, 1H), 7.40-7.35 (m, 1H), 7.18 (t, J=7.80 Hz, 2H), 6.91 (s, 1H), 6.83 (s, 1H), 4.28-4.4.22 (m, 1H), 3.74 (s, 3H), 2.17 (s, 3H), 0.80-0.70 (m, 4H) ppm.

### Example 11

### N-(3,5-Difluoro-pyridin-4-yl)-1-methyl-5-[4-methyl-6-(trifluoromethyloxy)-pyridin-3-yl]-1H-pyrazole-3-carboxylic acid amide

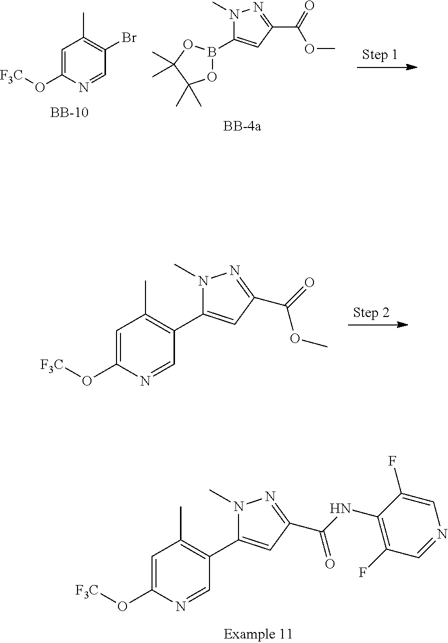

The title compound was prepared in analogy to example 9 starting from BB-10.

LC-MS (Method 2): m/z [M+H]⁺=414.1 (MW calc.=413.30); Rₜ=0.71 min.

¹H-NMR (DMSO-d₆): δ=10.45 (s, 1H), 8.60 (s, 2H), 8.37 (s, 1H), 7.40 (s, 1H), 6.99 (s, 1H), 3.78 (s, 3H), 2.28 (s, 3H) ppm.

### Example 12

### N-(2,6-Difluoro-phenyl)-1-methyl-5-[4-methyl-6-(trifluoromethyloxy)-pyridin-3-yl]-1H-pyrazole-3-carboxylic acid amide

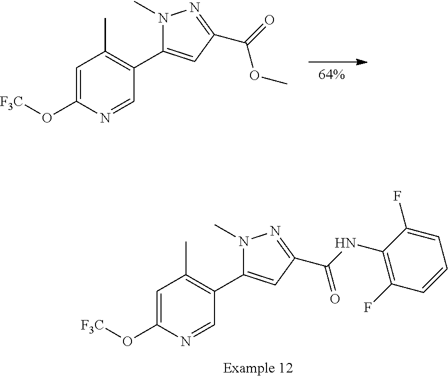 [SMILES: COC(=O)c1cc(-c2cnc(OC(F)(F)F)cc2C)n(C)n1 ; Cc1cc(OC(F)(F)F)ncc1-c1cc(C(=O)Nc2c(F)cccc2F)nn1C]

The title compound was prepared in analogy to example 9 starting from step 1 intermediate of example 11.

LC-MS (Method 2): m/z [M+H]⁺=413.1 (MW calc.=412.13); Rₜ=0.77 min.

¹H-NMR (DMSO-d₆): δ=9.95 (s, 1H), 8.36 (s, 1H), 7.42-7.35 (m, 2H), 7.18 (t, J=7.92 Hz, 2H), 6.93 (s, 1H), 3.77 (s, 3H), 2.28 (s, 3H) ppm.

### Example 13

### N-(2,6-Difluoro-phenyl)-5-(6-isopropoxy-4-methyl-pyridin-3-yl)-1-methyl-1H-pyrazole-3-carboxylic acid amide

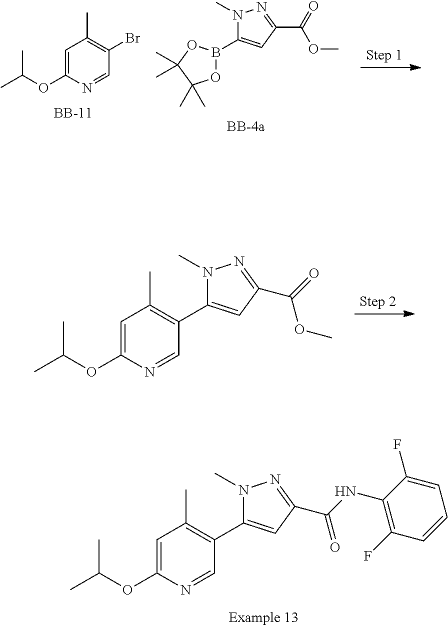

The title compound was prepared in analogy to example 9 starting from BB-11.

LC-MS (Method 2): m/z [M+H]⁺=387.2 (MW calc.=386.40); Rₜ=0.80 min.

¹H-NMR (DMSO-d₆): δ=9.87 (s, 1H), 8.09 (s, 1H), 7.42-7.35 (m, 1H), 7.21-7.14 (m, 2H), 6.81 (s, 1H), 6.79 (s, 1H), 5.33-5.26 (m, 1H), 3.74 (s, 3H), 2.14 (s, 3H), 1.32 (d, J=6.4 Hz, 6H) ppm.

### Example 14

### N-(3,5-Difluoro-pyridin-4-yl)-5-(6-ethoxy-4-methoxy-pyridin-3-yl)-1-methyl-1H-pyrazole-3-carboxylic acid amide/Example 15: 5-(4,6-Diethoxy-pyridin-3-yl)-N-(3,5-difluoro-pyridin-4-yl)-1-methyl-1H-pyrazole-3-carboxylic acid amide

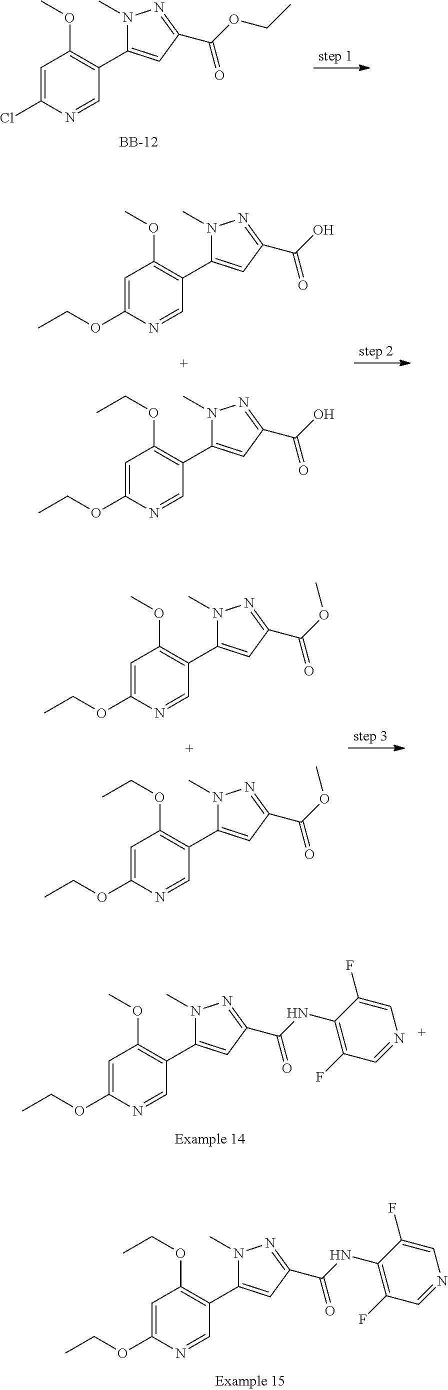

Step 1:

Sodium (194 mg, 8.42 mmol) was added to EtOH (42 mL) at 0° C. and stirred at RT for 30 min. BB-12 (1.24 g, 4.21 mmol) was added to the RM at RT and the mixture was heated to reflux for 16 h. The volatiles were removed under reduced pressure and the residue was diluted with water, was acidified with sat. NaHSO₄ solution to adjust the pH to −3-4 and was extracted with CH₂Cl₂. The combined organic layers were dried and the volatiles were removed under reduced pressure to get mixture a mixture of 5-(6-ethoxy-4-methoxy-pyridin-3-yl)-1-methyl-1H-pyrazole-3-carboxylic acid and 5-(4,6-diethoxy-pyridin-3-yl)-1-methyl-1H-pyrazole-3-carboxylic acid which was used in the next step without purification.

LC-MS (Method 3): m/z [M+H]⁺=278.2/292.3 (MW calc.=277.28/291.30); Rₜ=1.75/2.01 min.

Step 2:

To the crude mixture obtained in step 1 (1.1 g) in acetone (40 mL) was added K₂CO₃ (2.74 g, 19.9 mmol) at 0° C. followed by methyl iodide (0.75 mL, 11.9 mmol). The RM was stirred at RT for 16 h. The volatiles were removed under reduced pressure and the residue was diluted with EtOAc and was washed with water, brine and was dried. The volatiles were removed under reduced pressure to get crude mixture of 5-(6-ethoxy-4-methoxy-pyridin-3-yl)-1-methyl-1H-pyrazole-3-carboxylic acid methyl ester and 5-(4,6-diethoxy-pyridin-3-yl)-1-methyl-1H-pyrazole-3-carboxylic acid methyl ester which was used in the next step without further purification.

LC-MS (Method 3): m/z [M+H]⁺=292.0 and 306.0 (MW calc.=291.30 and 305.33; Rₜ=3.02 and 3.16 min.

Step 3:

To a solution of 3,5-difluoro-pyridin-4-ylamine (268 mg, 2.06 mmol) in toluene (10 mL) was added Me₃Al (1.55 mL, 3.09 mmol) at 0° C. and stirred for 15 min. A solution of the crude mixture obtained in step 2 (300 mg, 1.03 mmol) in toluene (2 mL) was added and the mixture was heated to reflux for 14 h. Sat. NH₄Cl was added and the mixture was extracted with CH₂Cl₂. The combined organic layers were washed with brine (75 ml), dried over Na₂SO₄ and concentrated under reduce pressure to get crude product which was purified by preparative HPLC to example compound 14 (95 mg, 23%) and example compound 15 (90 mg, 21%).

### Analytical Data for Example 14:

LC-MS (Method 2): m/z [M+H]⁺=390.1 (MW calc. 389.36); Rₜ=0.67 min.

¹H-NMR (DMSO-d₆, 400 MHz), δ=10.36 (s, 1H), 8.59 (s, 2H), 8.02 (s, 1H), 6.82 (s, 1H), 6.57 (s, 1H), 4.36 (q, J=7.0 Hz, 2H), 3.87 (s, 3H), 3.75 (s, 3H), 1.34 (t, J=7.0 Hz, 3H) ppm.

### Analytical Data for Example 15:

LC-MS (Method 2): m/z [M+H]⁺=404.2 (MW calc. 403.38); Rₜ=0.72 min.

¹H-NMR (DMSO-d₆, 400 MHz), δ=10.37 (s, 1H), 8.59 (s, 2H), 8.01 (s, 1H), 6.81 (s, 1H), 6.55 (s, 1H), 4.35 (q, J=7.1 Hz, 2H), 4.17 (q, J=7.0 Hz, 2H), 3.76 (s, 3H), 1.33 (t, J=7.1 Hz, 3H), 1.29 (t, J=7.0 Hz, 3H) ppm.

### Example 16

### N-(2,6-Difluoro-phenyl)-5-(6-ethoxy-4-methoxy-pyridin-3-yl)-1-methyl-1H-pyrazole-3-carboxylic acid amide/Example 17: 5-(4,6-Diethoxy-pyridin-3-yl)-N-(2,6-difluoro-phenyl)-1-methyl-1H-pyrazole-3-carboxylic acid amide

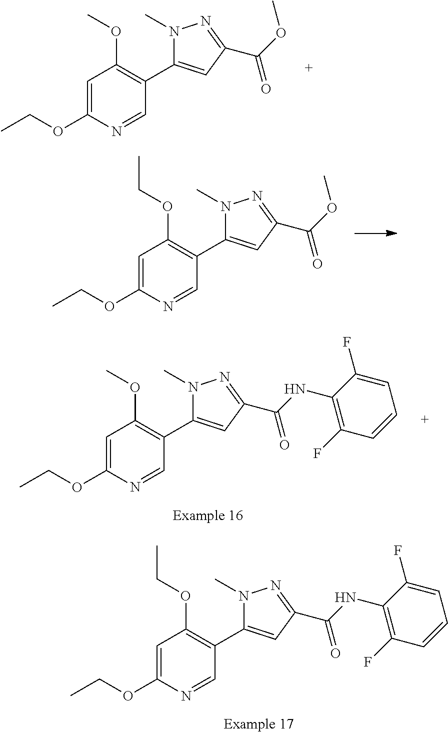

The title compounds of example 16 and 17 were prepared in analogy to examples 14 and 15.

### Analytical Data for Example 16:

LC-MS (Method 2): m/z [M+H]⁺=389.1 (MW calc. 389.36); Rₜ=0.74 min.

¹H-NMR (DMSO-d₆, 400 MHz), δ=9.86 (s, 1H), 8.01 (s, 1H), 7.40-7.36 (m, 1H), 7.18 (t, J=7.9 Hz, 2H), 6.76 (s, 1H), 6.57 (s, 1H), 4.36 (q, J=7.1 Hz, 2H), 3.87 (s, 3H), 3.73 (s, 3H), 1.34 (t, J=7.0 Hz, 3H) ppm.

### Analytical Data for Example 17:

LC-MS (Method 2): m/z [M+H]⁺=403.2 (MW calc. 402.39); Rₜ=0.79 min.

¹H-NMR (DMSO-d₆, 400 MHz), δ=9.87 (s, 1H), 8.01 (s, 1H), 7.40-7.32 (m, 1H), 7.18 (t, J=8.0 Hz, 2H), 6.76 (s, 1H), 6.54 (s, 1H), 4.35 (q, J=7.1 Hz, 2H), 4.17 (q, J=7.0 Hz, 2H), 3.75 (s, 3H), 1.33 (t, J=7.0 Hz, 3H), 1.29 (t, J=7.0 Hz, 3H) ppm.

### Example 18

### 5-(6-Cyclopropyloxy-4-methyl-pyridin-3-yl)-N-(3,5-difluoro-pyridin-4-yl)-1-methyl-1H-pyrazole-3-carboxylic acid amide

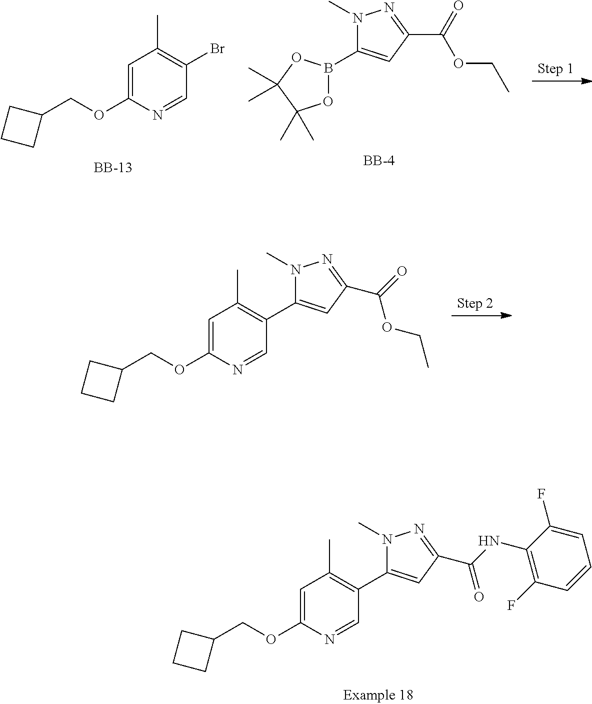

Step 1:

A mixture of BB-4 (1.31 g, 4.68 mmol), BB-13 (0.6 g, 2.34 mmol, LiOH (56 mg, 2.61 mmol) and bis(tri-tert-butylphosphine)palladium (71 mg, 0.16 mmol) in DMF (10 mL) was degassed through purging with N₂ and subsequently heated in the microwave to 80° C. for 1 h and to 100° C. for an additional hour. Purification by chromatography (SiO2, EtOAc/Hex) yielded the desired compound (0.68 g, 87%).

Step 2:

To a solution of the intermediate from step 1 (100 mg, 0.30 mmol) in THF (2.3 mL) was added under N₂ atmosphere 2,6-difluorophenylamine (77 mg, 0.61 mmol) and LHMDS (1M in THF, 0.76 mmol). The RM was heated in the microwave to 100° C. for 1 h before another equivalent of LHMDS was added and heating continued for 1 h. The RM was quenched with MeOH and purified by chromatography (SiO₂, EtOAc/Hex) to give the title compound (62 mg, 49%).

LC-MS (Method 2): m/z [M+H]⁺=413.3 (MW calc. 412.44); Rₜ=0.88 min.

¹H-NMR (DMSO-d₆): δ=9.87 (s, 1H), 8.09 (s, 1H), 7.40-7.35 (m, 1H), 7.18 (m, 2H), 6.86 (s, 1H), 6.82 (s, 1H), 4.28 (d, J=6.8 Hz, 2H), 3.73 (s, 3H), 2.74 (m, 1H), 2.15 (s, 3H), 2.08 (m, 2H), 1.81-1.93 (m, 4H) ppm.

### Example 19

### N-(2,6-Difluoro-phenyl)-1-methyl-5-[4-methyl-6-[(5-methyl-isoxazol-3-yl)-methoxy]-pyridin-3-yl]-1H-pyrazole-3-carboxylic acid amide

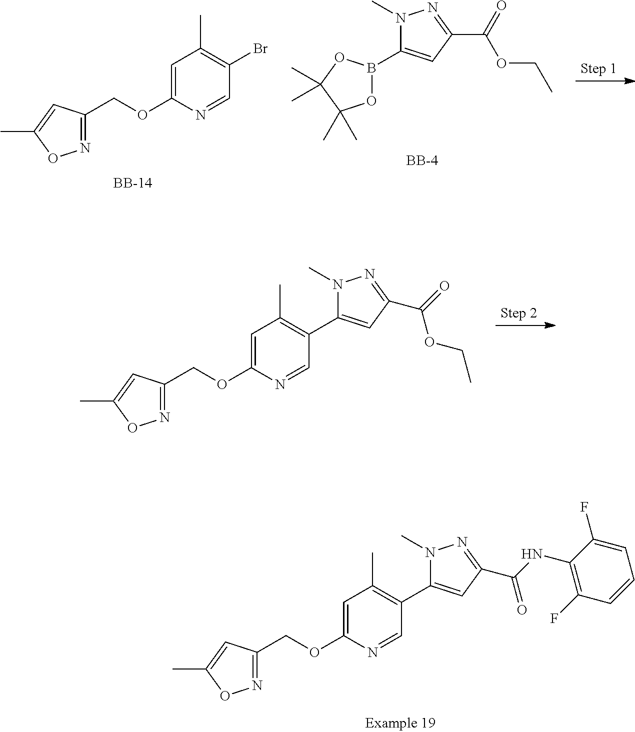

Step 1:

The preparation was performed in analogy to step 1 of example 18 employing BB-14 (0.21 g).

LC-MS (Method 2): m/z [M+H]⁺=357.3 (MW calc. 356.38); Rt=0.85 min.

Step 2:

The title compound was prepared in analogy to step 2 of example 18 (56 mg, 32% yield).

LC-MS (Method 2): m/z [M+H]⁺=440.3 (MW calc. 439.42); Rₜ=0.73 min.

¹H-NMR (DMSO-d₆): δ=9.88 (s, 1H), 8.15 (s, 1H), 7.40-7.35 (m, 1H), 7.18 (m, 2H), 6.95 (s, 1H), 6.84 (s, 1H), 6.31 (s, 1H), 5.42 (s, 2H), 3.74 (s, 3H), 2.41 (s, 3H), 2.18 (s, 3H).

### Example 20

### 5-[6-(Cyclobutyl-methoxy)-4-methyl-pyridin-3-yl]-N-(3,5-difluoro-pyridin-4-yl)-1-methyl-1H-pyrazole-3-carboxylic acid amide

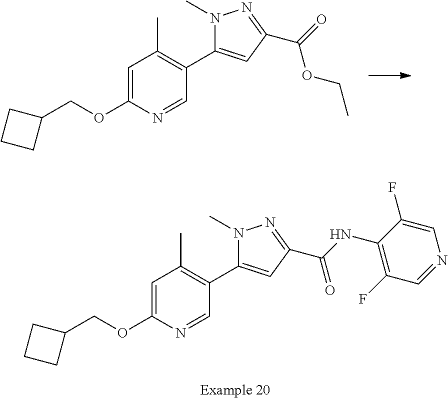 [SMILES: CCOC(=O)c1cc(-c2cnc(OCC3CCC3)cc2C)n(C)n1 ; Cc1cc(OCC2CCC2)ncc1-c1cc(C(=O)Nc2c(F)cncc2F)nn1C]

The title compound was prepared in analogy to example 18 employing the intermediate from step 1 and 4-amino-3,5-difluoropyridine (38 mg, 30%).

LC-MS (Method 2): m/z [M+H]⁺=414.2 (MW calc. 413.43); Rₜ=0.79 min.

¹H-NMR (DMSO-d₆): δ=10.37 (s, 1H), 8.59 (s, 2H), 8.10 (s, 1H) 6.87 (s, 1H), 6.86 (s, 1H), 4.29 (d, J=7.0 Hz, 2H), 3.75 (s, 3H), 2.74 (m, 1H), 2.15 (s, 3H), 2.08 (m, 2H), 1.81-1.93 (m, 4H) ppm.

### Example 21

### N-(2,6-Difluoro-phenyl)-5-[6-[2-(dimethylamino)ethyloxy]-4-methyl-pyridin-3-yl]-1-methyl-1H-pyrazole-3-carboxylic acid amide

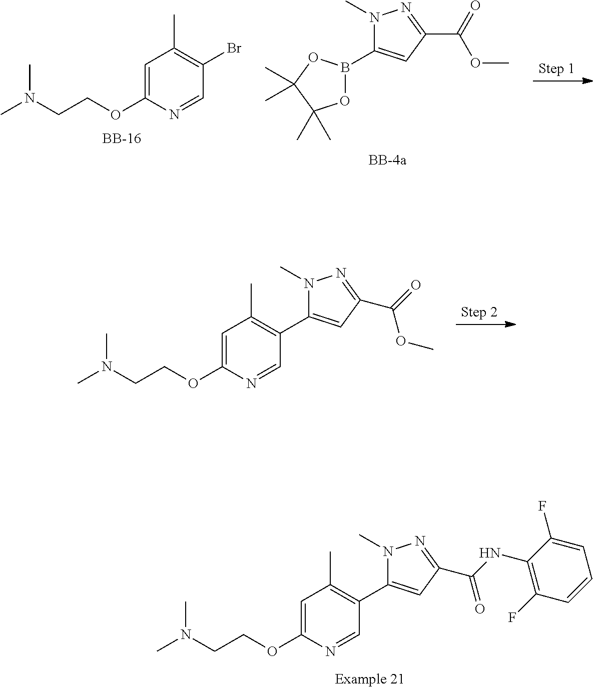

Step 1:

The preparation was performed in analogy to step 1 of example 18 employing BB-16 (0.20 g, 46% yield).

LC-MS (Method 3): m/z [M+H]⁺=319.3 (MW calc. 318.37); Rₜ=2.20 min.

Step 2:

To a solution of 2,6-difluoro-phenylamine (99 μl, 0.92 mmol) in toluene (3 mL) was added 2M solution of Me₃Al in toluene (0.939 mL, 1.87 mmol) at 0° C. and stirred for 30 min. A solution of the intermediate of step 1 (200 mg, 0.628 mmol) in toluene (2 mL) was added to the RM and stirred at RT for 14 h. The RM was quenched with sat. NH₄Cl solution and extracted with EtOAc. The combined organic layers were washed with water and brine, dried and the volatiles were removed under reduced pressure. The residue was purified by CC (SiO₂, MeOH/CH₂Cl₂) to yield the desired compound (99 mg, 38%).

LC-MS (Method 2): m/z [M+H]⁺=416.2 (MW calc.=415.44); Rₜ=0.43 min.

¹H-NMR (DMSO-d₆, 400 MHz), δ=9.90 (s, 1H), 8.10 (s, 1H), 7.42 (m, 1H), 7.18 (t, J=8.0 Hz, 2H), 6.86 (s, 1H), 6.82 (s, 1H), 4.39 (t, J=5.8 Hz, 2H), 3.73 (s, 3H), 2.68 (t, J=5.5 Hz, 2H), 2.25 (s, 6H), 2.15 (s, 3H) ppm.

### Example 22

### 5-(6-Chloro-4-methyl-pyridin-3-yl)-N-(2,6-difluoro-phenyl)-1-methyl-1H-pyrazole-3-carboxylic acid amide

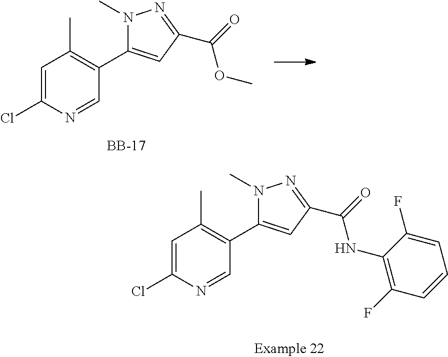

Example 22 was prepared in analogy to step 2 of example 21 employing BB-17 (0.25 g, 61% yield).

LC-MS (Method 3): m/z [M+H]⁺=363.0 (MW calc. 362.76); Rₜ=3.13 min.

### Example 23

### N-(2,6-Difluoro-phenyl)-1-methyl-5-[4-methyl-6-(oxetan-3-yloxy)-pyridin-3-yl]-1H-pyrazole-3-carboxylic acid amide

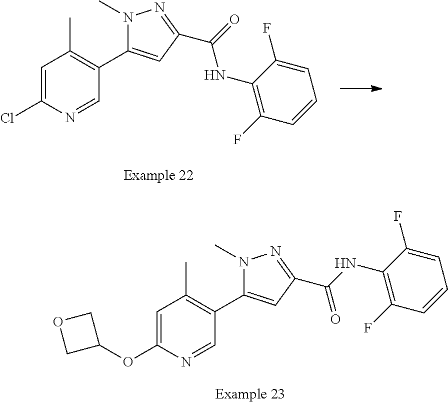 [SMILES: Cc1cc(Cl)ncc1-c1cc(C(=O)Nc2c(F)cccc2F)nn1C ; Cc1cc(OC2COC2)ncc1-c1cc(C(=O)Nc2c(F)cccc2F)nn1C]

To a solution of oxetan-3-ol (63 μL, 0.99 mmol) in NMP (1.5 mL) was added NaH (39 mg, 0.99 mmol, 60% in mineral oil) at 0° C. and the RM was stirred for 15 min followed by addition of a solution of the title compound of example 22 (120 mg, 0.33 mmol) in NMP (1 mL). The RM was warmed to RT and subsequently heated to 120° C. for 16 h. Water was added and the mixture was extracted with EtOAc. The combined organic layers were washed with water and brine, dried and the volatiles removed under reduced pressure. The residue was purified by CC (SiO₂, EtOAc/Hex) to give the title compound (70 mg, 53%).

LC-MS (Method 2): m/z [M+H]⁺=401.1 (MW calc. 400.38); Rₜ=0.66 min.

¹H-NMR (DMSO-d6), δ=9.88 (s, 1H), 8.08 (s, 1H), 7.39 (m, 1H), 7.18 (m, 2H), 6.97 (s, 1H), 6.83 (s, 1H), 5.61 (m, 1H), 4.91 (m, 2H), 5.60 (m, 2H), 3.73 (s, 3H), 2.18 (s, 3H) ppm.

### Example 24

### N-(2,6-Difluoro-phenyl)-5-(6-hydroxy-4-methyl-pyridin-3-yl)-1-methyl-1H-pyrazole-3-carboxylic acid amide

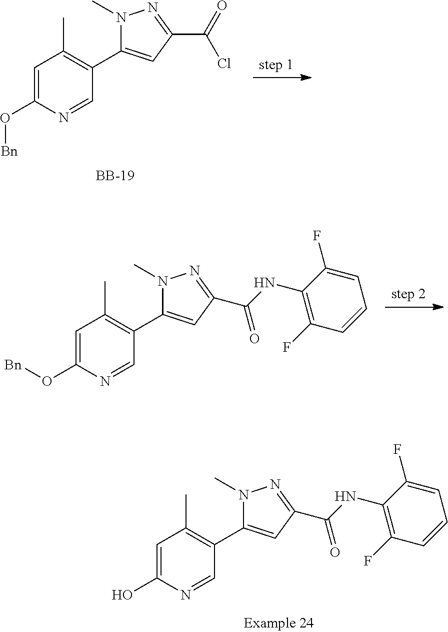 [SMILES: Cc1cc(OCc2ccccc2)ncc1-c1cc(C(=O)Cl)nn1C ; "BB-19" ; Cc1cc(OCc2ccccc2)ncc1-c1cc(C(=O)Nc2c(F)cccc2F)nn1C ; Cc1cc(O)ncc1-c1cc(C(=O)Nc2c(F)cccc2F)nn1C]

Step 1:

To a cooled (0° C.) solution of BB-19 (1.24 mmol) in CH₂Cl₂ (8 mL) were added DIPEA (0.66 ml, 3.72 mmol) and 2,6-difluoro-phenylamine (160 mg, 1.24 mmol) at 0° C. and the RM was stirred at RT for 14 h. The RM was diluted with CH₂Cl₂, washed with water, sat. NH₄Cl solution and brine. The organic layer was dried and concentrated under reduced pressure. The residue was purified by CC (SiO₂, EtOAc/Hex) to give the desired compound (200 mg, 37% yield).

Step 2:

A solution of the intermediate from step 1 (200 mg, 0.46 mmol) in MeOH (10 mL) was degassed with Ar for 15 min followed by addition of 10% Pd—C(100 mg). The RM was stirred under H₂ atmosphere at RT for 16 h. The RM was filtered through celite and the filtrate was concentrated under reduced pressure. The residue was triturated with CH₂Cl₂-Hex to give the title compound (97 mg, 61%).

LC-MS (Method 2): m/z [M+H]⁺=345.1 (MW calc. 344.32); Rt=0.47 min.

¹H-NMR (DMSO-d₆, 400 MHz), δ=11.78 (s, 1H), 9.86 (s, 1H), 7.45 (s, 1H), 7.40-7.36 (m, 1H), 7.17 (t, J=7.92 Hz, 2H), 6.76 (s, 1H), 6.33 (s, 1H), 3.75 (s, 3H), 1.96 (s, 3H).

### Alternatively the Following Route was Employed:

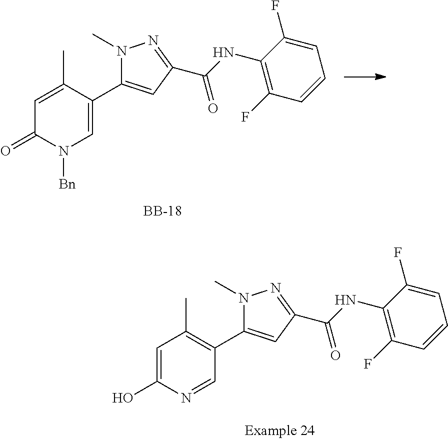 [SMILES: Cc1cc(=O)n(Cc2ccccc2)cc1-c1cc(C(=O)Nc2c(F)cccc2F)nn1C ; "BB-18" ; Cc1cc(O)ncc1-c1cc(C(=O)Nc2c(F)cccc2F)nn1C]

A solution of BB-18 (4.0 g, 9.21 mmol) in MeOH (50 mL) was degassed with N₂ for 15 min followed by the addition of Pd—C(2.0 g). The RM was stirred under H₂ atmosphere for 4 h and was than filtered over a pad of celite. The filtrate was concentrated under reduced pressure to afford the title compound (2.8 g, 88%) which was used as intermediate in next steps without further purification.

LC-MS (Method 3): m/z [M+H]⁺=345.2 (MW calc. 344.32); Rt=2.59 min.

### Example 25

### N-(3,5-Difluoro-pyridin-4-yl)-5-(6-hydroxy-4-methyl-pyridin-3-yl)-1-methyl-1H-pyrazole-3-carboxylic acid amide

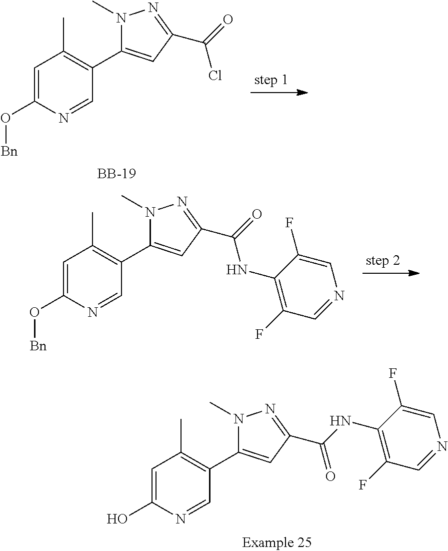

The title compound was prepared in analogy to example 24 starting from BB-19.

LC-MS (Method 2): m/z [M+H]⁺=346.1 (MW calc. 345.30); Rₜ=0.38 min.

¹H-NMR (DMSO-d₆), δ=11.79 (s, 1H), 10.36 (s, 1H), 8.59 (s, 2H), 7.46 (s, 1H), 6.82 (s, 1H), 6.33 (s, 1H), 3.76 (s, 3H), 1.96 (s, 3H) ppm.

### Example 26

### 2-[5-[5-[(2,6-Difluoro-phenyl)-carbamoyl]-2-methyl-2H-pyrazol-3-yl]-4-methyl-pyridin-2-yl]oxy-acetic acid ethyl ester

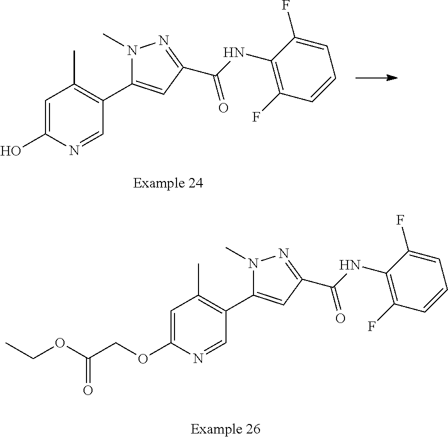 [SMILES: Cc1cc(O)ncc1-c1cc(C(=O)Nc2c(F)cccc2F)nn1C ; CCOC(=O)COc1cc(C)c(-c2cc(C(=O)Nc3c(F)cccc3F)nn2C)cn1]

A mixture of the title compound of example 26 (1.0 g, 2.90 mmol), ethyl bromoacetate (356 μl, 3.19 mmol) and silver oxide (1.33 g, 5.8 mmol) in CH₃CN (20 mL) was heated to 60° C. for 16 h. The RM was filtered over a pad of celite and the filtrate was concentrated under reduced pressure and purified by CC (SiO₂, EtOAc/Hex) to afford the title compound (430 mg, 34%).

LC-MS (Method 3): m/z [M+H]⁺=431.1 (MW calc. 430.40); Rₜ=3.35 min.

### Example 27

### 2-[5-[5-[(2,6-Difluoro-phenyl)-carbamoyl]-2-methyl-2H-pyrazol-3-yl]-4-methyl-pyridin-2-yl]oxy-acetic acid

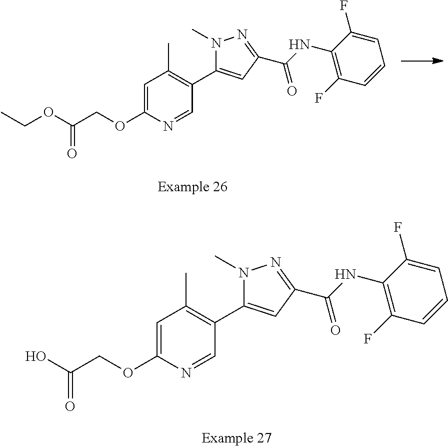 [SMILES: CCOC(=O)COc1cc(C)c(-c2cc(C(=O)Nc3c(F)cccc3F)nn2C)cn1 ; Cc1cc(OCC(=O)O)ncc1-c1cc(C(=O)Nc2c(F)cccc2F)nn1C]

To a solution of the title compound of example 26 (100 mg, 232 μmol) in a mixture of MeOH (1 mL) and THF (1 mL) was added a solution of LiOH.H₂O (19.5 mg, 465 μmol) in water (1 mL) at 0° C. and the RM was stirred at RT for 4 h. The volatiles were removed under reduced pressure and the residue was diluted with water. The aqueous layer was acidified to pH≈3-4 using NaHSO₄ solution and was extracted with CH₂Cl₂. The combined organic layers were dried and the volatiles were removed to yield the desired compound (80 mg, 86%).

LC-MS (Method 2): m/z [M+H]⁺=403.2 (MW calc. 402.35); Rₜ=0.57 min.

¹H-NMR (DMSO-d₆), δ=12.86 (s, 1H), 9.91 (s, 1H), 8.09 (s, 1H), 7.41-7.35 (m, 1H), 7.18 (t, J=8.0 Hz, 2H), 6.96 (s, 1H), 6.83 (s, 1H), 4.86 (s, 2H), 3.73 (s, 3H), 2.18 (s, 3H) ppm.

### Example 28

### N-(2,6-Difluoro-phenyl)-5-[6-(2-methoxy-ethoxy)-4-methyl-pyridin-3-yl]-1-methyl-1H-pyrazole-3-carboxylic acid amide

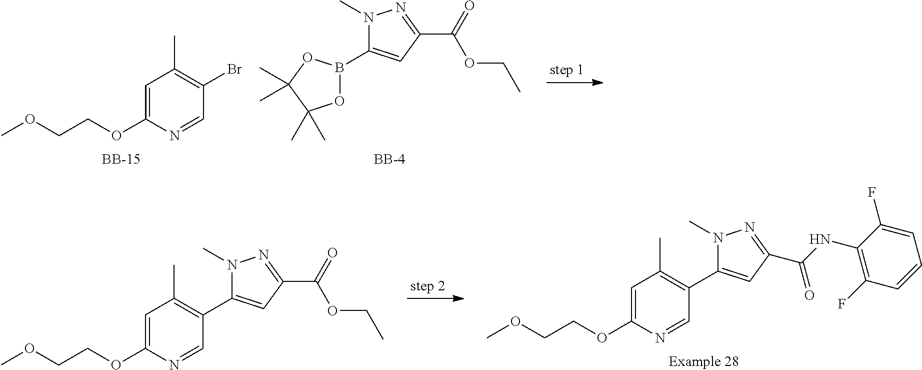

Step 1:

The preparation was performed in analogy to step 1 of example 18 employing BB-15 (0.32 g, 43%).

LC-MS (Method 2): m/z [M+H]=320.2 (MW calc. 319.61); Rt=0.64 min.

Step 2:

The title compound was prepared in analogy to step 2 of example 18 (122 mg, 26%).

LC-MS (Method 2): m/z [M+H]⁺=403.3 (MW calc. 402.40); Rₜ=0.71 min.

¹H-NMR (DMSO-d₆): δ=9.88 (s, 1H), 8.10 (s, 1H), 7.40-7.35 (m, 1H), 7.18 (m, 2H), 6.88 (s, 1H), 6.83 (s, 1H), 4.43 (t, J=4.5 Hz, 2H), 3.74 (s, 3H), 2.68 (t, J=4.5 Hz, 2H), 2.16 (s, 3H) ppm.

### Example 29

### N-(2,6-Difluoro-phenyl)-5-[6-[(dimethyl-carbamoyl)-methoxy]-4-methyl-pyridin-3-yl]-1-methyl-1H-pyrazole-3-carboxylic acid amide

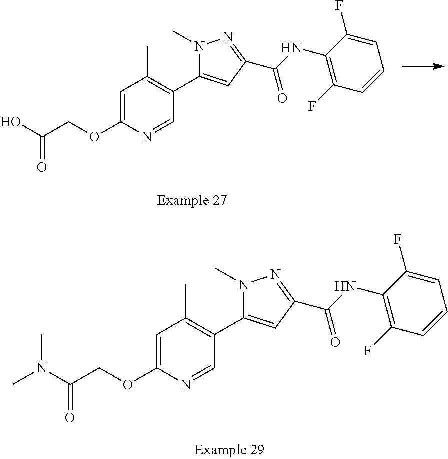 [SMILES: Cc1cc(OCC(=O)O)ncc1-c1cc(C(=O)Nc2c(F)cccc2F)nn1C ; Cc1cc(OCC(=O)N(C)C)ncc1-c1cc(C(=O)Nc2c(F)cccc2F)nn1C]

To a solution of the title compound of example 27 (150 mg, 373 μmol) in CH₂Cl₂ were added DIPEA (0.19 mL, 1.11 mmol), HATU (141 mg, 0.373 mmol) and dimethylamine (2M in THF, 270 μL, 559 μmol) at RT and the RM was stirred for 16 h. The RM was diluted with CH₂Cl₂ and was washed with sat. NH₄Cl and brine. The organic layer was dried, the volatiles were removed under reduced pressure and the residue was purified by CC (SiO₂, MeOH/CH₂Cl₂) to give the title compound (95 mg, 59%).

LC-MS (Method 2): m/z [M+H]⁺=430.2 (MW calc. 429.42); Rₜ=0.60 min.

¹H-NMR (DMSO-d₆), δ=9.91 (s, 1H), 8.05 (s, 1H), 7.42-7.35 (m, 1H), 7.18 (t, J=8.0 Hz, 2H), 6.94 (s, 1H), 6.82 (s, 1H), 5.08 (s, 2H), 3.73 (s, 3H), 2.99 (s, 3H), 2.83 (s, 3H), 2.17 (s, 3H) ppm.

### Example 30

### 5-[6-(Carbamoyl-methoxy)-4-methyl-pyridin-3-yl]-N-(2,6-difluoro-phenyl)-1-methyl-1H-pyrazole-3-carboxylic acid amide

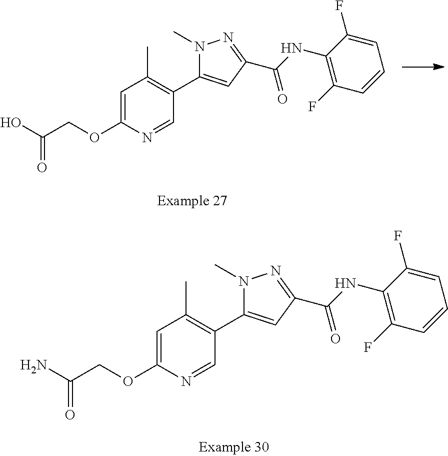 [SMILES: Cc1cc(OCC(=O)O)ncc1-c1cc(C(=O)Nc2c(F)cccc2F)nn1C ; Cc1cc(OCC(N)=O)ncc1-c1cc(C(=O)Nc2c(F)cccc2F)nn1C]

To a solution of the title compound of example 27 (190 mg, 473 μmol) in THF (5 mL) were added HOBT-NH₃ (108 mg, 0.71 mmol), DIPEA (0.24 mL, 1.41 mmol) and EDCl (135 mg, 0.71 mmol) at RT and the RM was stirred for 16 h. The RM was diluted with CH₂Cl₂ and was washed with sat. NH₄Cl solution and brine. The organic layer was dried, the volatiles were removed under reduced pressure and the residue was purified by chromatography (SiO₂, CH₂Cl₂/MeOH) to yield the title compound (90 mg, 47%).

LC-MS (Method 2): m/z [M+H]⁺=402.1 (MW calc. 401.37); Rₜ=0.53 min.

¹H-NMR (DMSO-d₆), δ=9.92 (s, 1H), 8.09 (s, 1H), 7.47 (s, 1H), 7.42-7.35 (m, 1H), 7.20-7.16 (m, 3H), 6.94 (s, 1H), 6.83 (s, 1H), 4.71 (s, 2H), 2.96 (s, 3H), 2.17 (s, 3H) ppm.

### Example 31

### N-(3,5-Difluoro-pyridin-4-yl)-5-[6-ethoxy-4-(trifluoromethyl)-pyridin-3-yl]-1-methyl-1H-pyrazole-3-carboxylic acid amide

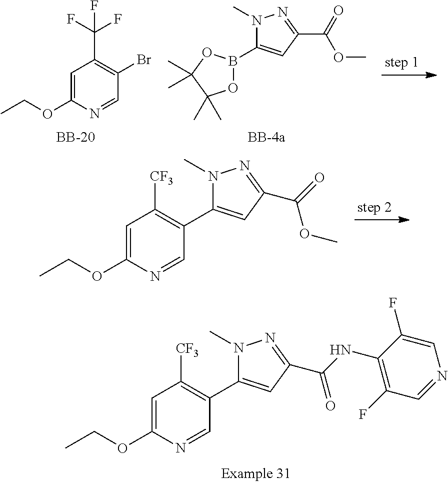

Step 1:

A mixture of BB-20 (500 mg, 1.85 mmol), BB-4a (640 mg, 2.40 mmol) and LiOH (47 mg, 1.97 mmol) in DMF (5 mL) was degassed with Ar for 15 min. Bis-(tri-tert-butyl phosphine)palladium (47 mg, 0.092 mmol) was added to the RM and heated at 90° C. for 1 h. The RM was diluted with EtOAc and washed with water and brine. The organic layer was dried, concentrated under reduced pressure and purified by CC (SiO₂, EtOAc/Hex) to give the desired compound (220 mg, 36%).

LC-MS (Method 3): m/z [M+H]⁺=330.0 (MW calc. 329.27); Rₜ=3.43 min.

Step 2:

To a solution of 3,5-difluoro-pyridin-4-ylamine (71 mg, 0.547 mmol) in toluene (3 mL) was added a 2M solution of Me₃Al in toluene (0.546 ml, 1.09 mmol) at 0° C. and stirred for 15 min. A solution of the intermediate from step 1 (120 mg, 0.364 mmol) in toluene (2 mL) was added to the RM and heated to reflux for 14 h. The RM was quenched with 1N HCl and extracted with EtOAc. The combined organic layers were washed with water and brine, dried and the volatiles evaporated under reduced pressure. The residue was purified by CC (SiO₂, EtOAc/Hex) to give the title compound (68 mg, 43%).

LC-MS (Method 2): m/z [M+H]⁺=428.1 (MW calc. 427.33); Rₜ=0.76 min.

¹H-NMR (DMSO-d₆) δ=10.46 (s, 1H), 8.60 (s, 2H), 8.46 (s, 1H), 7.36 (s, 1H), 6.90 (s, 1H), 4.44 (q, J=7.0 Hz, 2H), 3.73 (s, 3H), 1.38 (t, J=7.0 Hz, 3H) ppm.

### Example 32

### N-(2,6-Difluoro-phenyl)-5-[6-ethoxy-4-(trifluoromethyl)-pyridin-3-yl]-1-methyl-1H-pyrazole-3-carboxylic acid amide

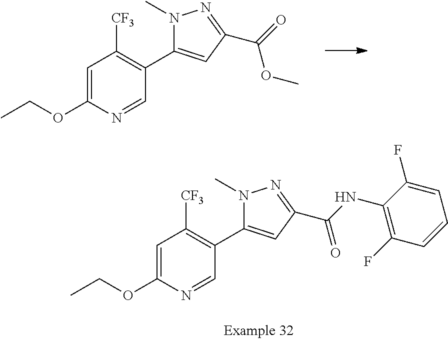

To a solution of 2,6-difluoro-phenylamine (49 μL, 0.455 mmol) in toluene (1 mL) was added Me₃Al (2M in toluene, 454 μL, 909 μmol) at 0° C. and the mixture was stirred for 15 min. A solution of step 1 intermediate of example 31 (100 mg, 303 μmol) in toluene (2 mL) was added and the RM was stirred at RT for 14 h. Aqueous HCl (1 M) was added the mixture was extracted with EtOAc. The combined organic layers were washed with water and brine, dried and the volatiles were removed under reduced pressure. The residue was purified by chromatography (SiO₂, EtOAc/Hex) to yield the title compound (79 mg, 61%).

LC-MS (Method 2): m/z [M+H]⁺=427.1 (MW calc. 426.34); Rₜ=0.81 min.

¹H-NMR (DMSO-d₆), δ=9.95 (s, 1H), 8.46 (s, 1H), 7.43-7.35 (m, 2H), 7.18 (t, J=8.0 Hz, 2H), 6.84 (s, 1H), 4.44 (q, J=7.0 Hz, 2H), 3.71 (s, 3H), 1.38 (t, J=7.0 Hz, 3H) ppm.

### Example 33

### 5-(6-Cyclopropyl-5-fluoro-4-methyl-pyridin-3-yl)-N-(2,6-difluoro-phenyl)-1-methyl-1H-pyrazole-3-carboxylic acid amide

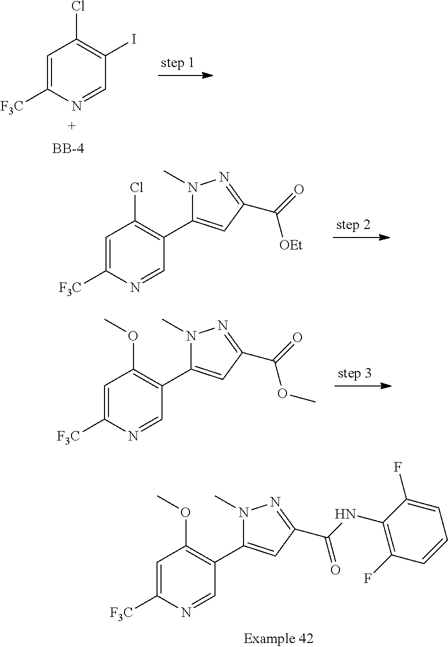

Step 1:

A mixture of BB-4a (981 mg, 3.69 mmol), 2-chloro-3-fluoro-5-iodo-4-methyl-pyridine (1.0 g, 3.69 mmol) and LiOH (94 mg, 3.94 mmol) in DMF (5 mL) was degassed through purging with Ar for 15 min. Bis-(tri-tert-butyl phosphine)palladium (94 mg, 184 μmol) was added and the RM was heated to 90° C. for 1.5 h. The RM was diluted with EtOAc and was washed with water, brine, was dried and the volatiles were removed under reduced pressure. The residue was purified by chromatography (SiO₂, EtOAc/Hex) to yield the desired compound (600 mg, 57%).

LC-MS (Method 1): m/z [M+H]⁺=283.8 (MW calc.=283.69); Rₜ=3.08 min.

Step 2:

A mixture of the intermediate of step 1 (600 mg, 2.12 mmol), potassium cyclopropyltrifluoroborate (470 mg, 3.18 mmol) and Cs₂CO₃ (3.4 g, 10.6 mmol) in toluene (12 mL) and water (5 mL) was degassed through purging with Ar for 15 min. Pd(OAc)₂ (47 mg, 212 μmol) and di(1-adamantyl)-n-butylphosphine (151 mg, 424 mmol) were added and the RM was heated to reflux for 16 h. The RM was diluted with EtOAc, was washed with water, brine, was dried and the volatiles were removed under reduced. The residue was purified by chromatography (SiO₂, EtOAc/Hex) to yield the desired compound (250 mg, 40%).

LC-MS (Method 1): m/z [M+H]⁺=290.2 (MW calc.=289.30); Rₜ=3.25 min.

Step 3:

To a solution of 2,6-difluoro-phenylamine (140 μl, 1.29 mmol) in toluene (3 mL) was added Me₃Al (2M in toluene, 1.29 mL, 2.59 mmol) at 0° C. and the RM was stirred for 15 min. A solution of the intermediate of step 2 (250 mg, 865 μmol) in toluene (2 mL) was added and the RM was stirred at RT for 14 h. Aqueous HCl (1N) was added and the mixture was extracted with EtOAc. The combined organic layers were washed with water and brine, were and the volatiles were removed under reduced pressure. The residue was purified by preparative HPLC to the title compound (80 mg, 24%).

LC-MS (Method 2): m/z [M+H]⁺=387.1 (MW calc.=386.37); Rₜ=0.78 min.

¹H-NMR (DMSO-d₆), δ (ppm)=9.95 (s, 1H), 8.24 (s, 1H), 7.43-7.35 (m, 1H), 7.18 (t, J=8.0 Hz, 2H), 6.88 (s, 1H), 3.76 (s, 3H), 2.37-2.34 (m, 1H), 2.15 (d, J=1.6 Hz, 3H), 1.10-1.05 (m, 4H) ppm.

### Example 34

### 5-(6-Cyclopropyl-5-fluoro-4-methyl-pyridin-3-yl)-N-(3,5-difluoro-pyridin-4-yl)-1-methyl-1H-pyrazole-3-carboxylic acid amide

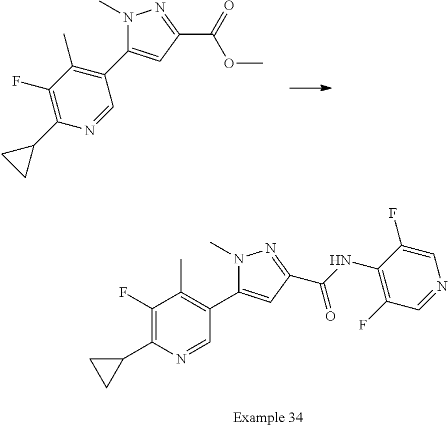 [SMILES: COC(=O)c1cc(-c2cnc(C3CC3)c(F)c2C)n(C)n1 ; Cc1c(-c2cc(C(=O)Nc3c(F)cncc3F)nn2C)cnc(C2CC2)c1F]

The title compound was prepared in analogy to example 9 starting from step 2 intermediate of example 33 (56 mg, 21%).

LC-MS (Method 2): m/z [M+H]⁺=388.1 (MW calc.=387.36; Rₜ=0.73 min.

¹H-NMR (DMSO-d₆), δ=10.45 (s, 1H), 8.60 (s, 2H), 8.24 (s, 1H), 6.94 (s, 1H), 3.77 (s, 3H), 2.38-2.32 (m, 1H), 2.15 (s, 3H), 1.08-1.05 (m, 4H) ppm.

### Example 35

### N-(2,6-Difluoro-phenyl)-5-(6-ethoxy-5-fluoro-4-methyl-pyridin-3-yl)-1-methyl-1H-pyrazole-3-carboxylic acid amide

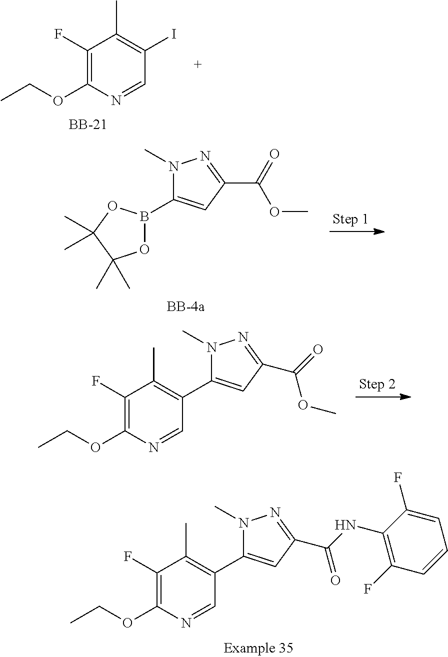

The title compound was prepared in analogy to example 9 starting from BB-21.

LC-MS (Method 2): m/z [M+H]⁺=391.1 (MW calc.=390.36); Rₜ=0.77 min.

¹H-NMR (DMSO-d₆), δ=9.93 (s, 1H), 7.98 (s, 1H), 7.40-7.37 (m, 1H), 7.18 (t, J=8.0 Hz, 2H), 6.86 (s, 1H), 4.46 (q, J=7.0 Hz, 2H), 3.76 (s, 3H), 2.11 (d, J=2.0 Hz, 3H), 1.38 (t, J=7.0 Hz, 3H) ppm.

### Example 36

### N-(3,5-Difluoro-pyridin-4-yl)-5-(6-ethoxy-5-fluoro-4-methyl-pyridin-3-yl)-1-methyl-1H-pyrazole-3-carboxylic acid amide

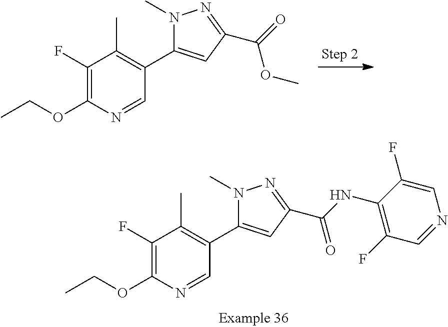

The title compound was prepared in analogy to example 9 starting from step 1 intermediate of example 35.

LC-MS (Method 2): m/z [M+H]⁺=392.1 (MW calc.=391.35); Rₜ=0.72 min.

¹H-NMR (DMSO-d₆), δ=9.93 (s, 1H), 7.98 (s, 1H), 7.40-7.37 (m, 1H), 7.18 (t, J=8.0 Hz, 2H), 6.86 (s, 1H), 4.46 (q, J=7.0 Hz, 2H), 3.76 (s, 3H), 2.11 (d, J=2.0 Hz, 3H), 1.38 (t, J=7.0 Hz, 3H) ppm.

### Example 37

### N-(2,6-Difluoro-phenyl)-5-(6-dimethylamino-4-methyl-pyridin-3-yl)-1-methyl-1H-pyrazole-3-carboxylic acid amide

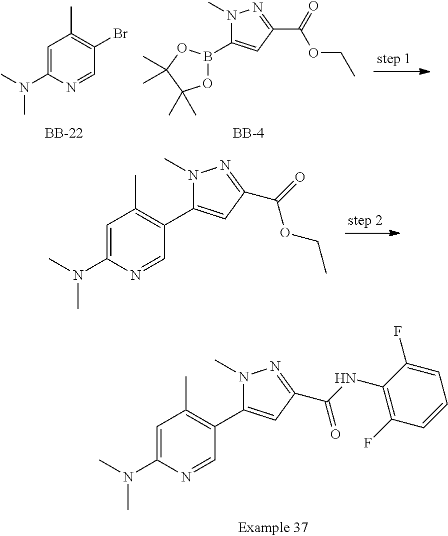

The title compound was prepared in analogy to example 9 starting from BB-22.

LC-MS (Method 2): m/z [M+H]⁺=372.2 (MW calc. 371.38); Rₜ=0.43 min.

¹H-NMR (DMSO-d₆): δ=9.92 (s, 1H), 7.98 (s, 1H), 7.43-7.36 (m, 1H), 7.21-7.15 (m, 2H), 6.74 (s, 1H), 6.64 (s, 1H), 3.72 (s, 3H), 3.07 (s, 6H), 2.11 (s, 3H) ppm.

### Example 38

### N-(2,6-Difluoro-phenyl)-5-(4,6-dimethoxy-pyridin-3-yl)-1-methyl-1H-pyrazole-3-carboxylic acid amide

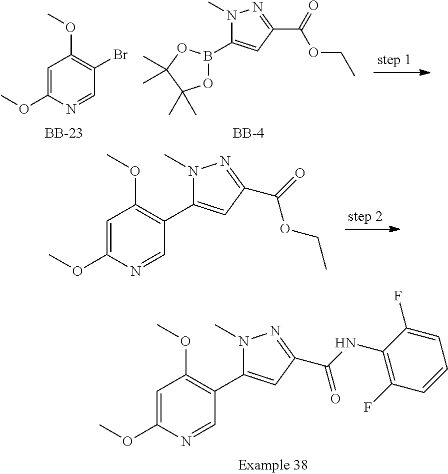

The title compound was prepared in analogy to example 9 starting from BB-23.

LC-MS (Method 2): m/z [M+H]⁺=375.1 (MW calc. 374.34); Rₜ=0.65 min.

¹H-NMR (DMSO-d₆): δ=9.83 (s, 1H), 8.04 (s, 1H), 7.42-7.36 (m, 1H), 7.20-7.14 (m, 2H), 6.77 (s, 1H), 6.59 (s, 1H), 3.91 (s, 3H), 3.88 (s, 1H), 3.74 (s, 1H) ppm.

### Example 39

### 5-(5-Chloro-2-methyl-pyridin-3-yl)-N-(3-fluoro-pyridin-4-yl)-1-methyl-1H-pyrazole-3-carboxylic acid amide

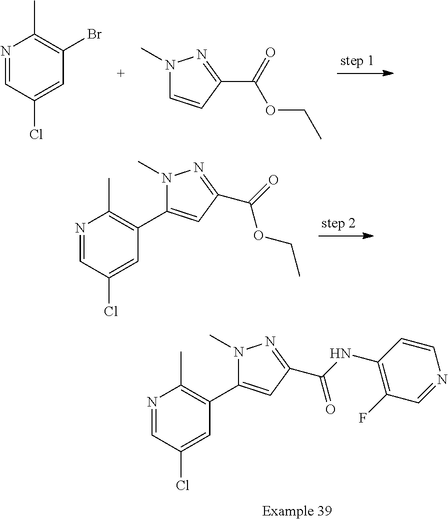

Step 1:

Palladium acetate (22 mg, 10 μmol) was added to a degassed solution of 3-bromo-5-chloro-2-methylpyridine (206 mg, 1.00 mmol) and ethyl 1-methyl-1H-pyrazole-3-carboxylate (154 mg, 1.00 mmol) and potassium acetate (200 mg, 2.00 mmol) in dry DMA (3 mL) and the resulting mixture was heated to 150° C. for 1.5 h. The volatiles were removed under reduced pressure and the residue was dissolved with brine and was extracted with EtOAc. The combined organic layers were dried and the volatiles were removed under reduced pressure. The residue was purified by chromatography (SiO₂, Cy/EtOAc) to yield the desired compound (130 mg, 46%).

¹H-NMR (CDCl₃): δ=8.57 (d, J=2.4 Hz, 1H), 7.53 (d, J=8.8 Hz, 1H), 6.81 (s, 1H), 4.43 (q, J=7.0 Hz, 2H), 4.43 (q, J=7.0 Hz, 2H), 3.75 (s, 3H), 2.37 (s, 3H), 1.41 (t, J=7.0 Hz, 3H) ppm.

Step 2:

A solution of the intermediate of step 1 (120 mg, 430 μmol) and 4-amino-3-fluoropyridine (63 mg, 560 μmol) in dry THF (4 mL) was treated with LHMDS (1M in Hex, 0.65 mL, 650 μmol) and the resulting mixture was stirred at 60° C. for 1 h. Water was added and the mixture was extracted with EtOAc. The combined organic layers were dried and the volatiles were removed under reduced pressure. The residue was purified through washing with Et₂O to yield the desired compound (93 mg, 63%).

LC-MS (Method 2): m/z [M+H]⁺=346.1 (MW calc. 345.76); Rₜ=0.63 min.

¹H-NMR (CDCl₃): δ=9.16 (s, 1H), 8.61 (d, J=2.4 Hz, 1H), 8.54 (m, 1H), 8.48 (s, 1H), 8.39 (d, J=5.6 Hz, 1H), 7.56 (d, J=2.4 Hz, 1H), 6.92 (s, 1H), 3.77 (s, 3H), 2.40 (s, 3H) ppm.

### Example 40

### 5-(5-Chloro-2-methyl-pyridin-3-yl)-N-(2,6-difluoro-phenyl)-1-methyl-1H-pyrazole-3-carboxylic acid amide

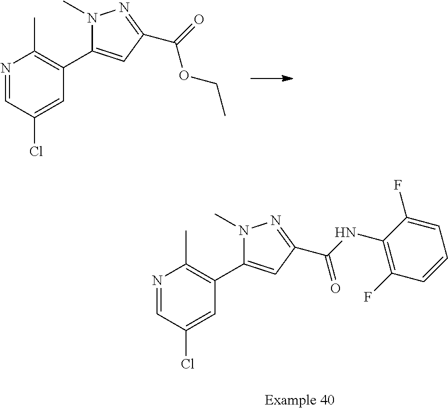 [SMILES: CCOC(=O)c1cc(-c2cc(Cl)cnc2C)n(C)n1 ; Cc1ncc(Cl)cc1-c1cc(C(=O)Nc2c(F)cccc2F)nn1C]

The title compound was prepared in analogy to example 9 starting from step 1 intermediate of example 35 (95 mg, 0.33 mmol) (108 mg, 97%).

LC-MS (Method 2): m/z [M+H]⁺=363.1 (MW calc. 362.76); Rₜ=0.67 min.

¹H-NMR (DMSO-d₆): δ=9.92 (s, 1H), 8.65 (d, J=2.3 Hz, 1H), 8.02 (d, J=2.3 Hz, 1H), 7.42-7.36 (m, 1H), 7.18 (t, J=7.9 Hz, 2H), 6.93 (s, 1H), 3.77 (s, 3H), 2.38 (s, 3H) ppm.

### Example 41

### 5-(6-Cyano-4-methyl-pyridin-3-yl)-N-(2,6-difluoro-phenyl)-1-methyl-1H-pyrazole-3-carboxylic acid amide

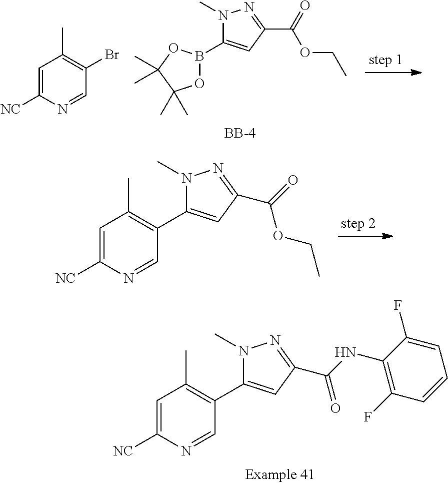

The title compound was prepared in analogy to example 9 starting from 5-bromo-4-methylpicolinonitrile.

LC-MS (Method 2): m/z [M+H]⁺=354.1 (MW calc.=353.33); Rₜ=0.61 min.

¹H-NMR (DMSO-d₆): δ=9.95 (s, 1H), 8.73 (s, 1H), 8.16 (s, 1H), 7.43-7.36 (m, 1H), 7.21-7.15 (m, 2H), 7.00 (s, 1H), 3.79 (s, 3H), 2.32 (s, 3H) ppm.

### Example 42

### N-(2,6-Difluoro-phenyl)-5-[4-methoxy-6-(trifluoromethyl)-pyridin-3-yl]-1-methyl-1H-pyrazole-3-carboxylic acid amide

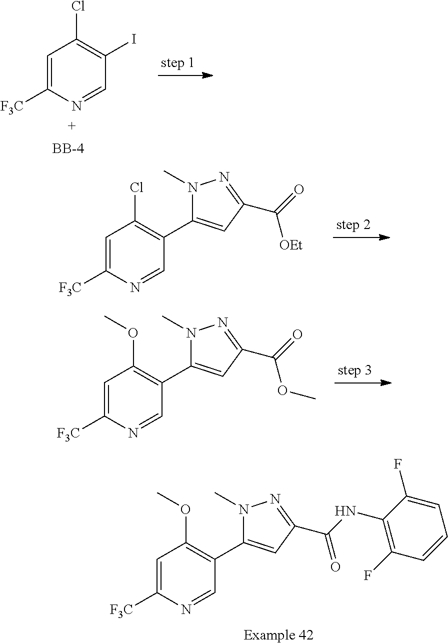

Step 1:

A degassed mixture of 4-chloro-5-iodo-2-(trifluoromethyl)pyridine (249 mg, 813 μmol), BB-4 (225 mg, 813 μmol), LiOH (20 mg, 906 μmol) and bis(tri-tert-butylphosphine)palladium (26 mg, 57 μmol) in DMF (3.3 mL) was heated to 80° C. for 1 h. The volatiles were removed under reduced pressure and the residue was purified by chromatography (Interchim® cartridge50SiHP/12 g, Cy/EtOAc) to yield the desired compound (191 mg, 70%).

LC-MS (Method 2): m/z [M+H]⁺=334.2 (MW calc.=333.69); Rₜ=0.87 min.

Step 2:

Sodium (16 mg, 719 μmol) was dissolved in MeOH (0.4 mL) at 50° C. and the intermediate of step 1 (240 mg, 719 μmol) was added. The RM was stirred at 50° C. for 90 min, the mixture was chilled and HCl (1M) was added to adjust the pH to 7. The mixture was dissolved in EtOAc and was washed with brine, was dried and the volatiles were removed under reduced pressure to get the desired compound which was used for the next step without further purification (80 mg, 35%) (Note: a trans esterification to the methyl ester was obtained).

Step 3:

To a solution of the intermediate of step 2 (80 mg, 250 μmol) and 2,6-difluoroaniline (49 mg, 381 μmol) in THF (2 mL) was added LHMDS (1M in THF, 0.39 mL, 390 μL) and the mixture was heated under microwave conditions to 100° C. for 2 h. The mixture was quenched with MeOH and the volatiles were removed under reduced pressure. The residue was purified by chromatography (Interchim® cartridge30SiHP/4 g, Cy/EtOAc) to yield the desired compound (18 mg, 17%).

LC-MS (Method 2): m/z [M+H]⁺=413.1 (MW calc.=412.31); Rₜ=0.69 min.

¹H-NMR (DMSO-d₆): δ=9.92 (s, 1H), 8.64 (s, 1H), 7.72 (s, 1H), 7.40 (tt, J=8.4 Hz, 6.2 Hz, 1H), 7.19 (t, J=8.0 Hz, 2H), 6.95 (s, 1H), 4.06 (s, 3H), 3.81 (s, 3H) ppm.

### Example 43

### 5-[6-(Difluoro-methoxy)-4-methoxy-pyridin-3-yl]-N-(2,6-difluoro-phenyl)-1-methyl-1H-pyrazole-3-carboxylic acid amide

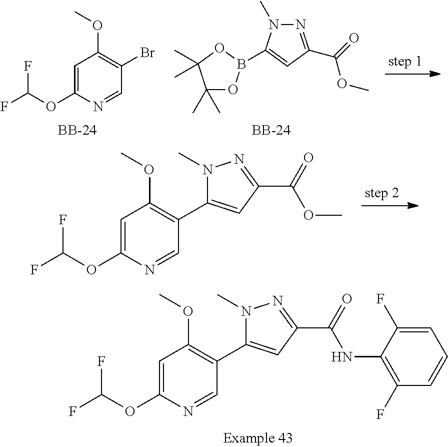

The title compound was synthesized in analogy to example 42 in 2 steps starting from BB-24 (300 mg, 1.18 mmol) and BB-4a (343 mg, 1.29 mmol) (68 mg, 15% over 2 steps).

LC-MS (Method 2): m/z [M+H]⁺=411.2 (MW calc.=410.32); Rt=0.71 min.

¹H-NMR (DMSO-d6, 400 MHz), δ (ppm)=9.88 (s, 1H), 8.15 (s, 1H), 7.96-7.60 (t, J=72.6 Hz, 1H), 7.40-7.35 (m, 1H), 7.18 (t, J=8.04 Hz, 2H), 6.94 (s, 1H), 6.83 (s, 1H), 3.94 (s, 3H), 3.76 (s, 3H).

### Example 44

### 5-[6-(Difluoro-methoxy)-4-methoxy-pyridin-3-yl]-N-(3,5-difluoro-pyridin-4-yl)-1-methyl-1H-pyrazole-3-carboxylic acid amide

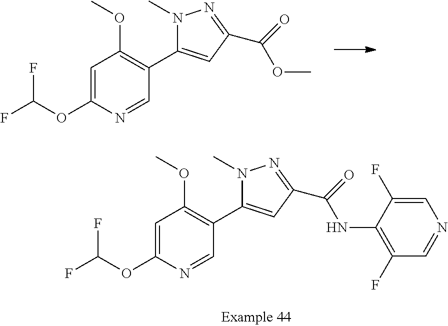 [SMILES: COC(=O)c1cc(-c2cnc(OC(F)F)cc2OC)n(C)n1 ; COc1cc(OC(F)F)ncc1-c1cc(C(=O)Nc2c(F)cncc2F)nn1C]

To a cooled a solution of step 1 intermediate of example 43 (120 mg, 0.38 mmol) and 3,5-difluoro-pyridin-4-ylamine (64 mg, 0.49 mmol) in THF (4 mL) was added LiHMDS (1M in THF, 0.45 mL, 0.45 mmol) and the RM was heated at reflux for 1 h. The RM was treated with with sat.NH₄Cl solution and was extracted with EtOAc. The combined organic layers were washed with water and brine, were dried and the volatiles were removed under reduced pressure to get crude product which was purified by CC (SiO₂, EtOAc/CH₂Cl₂) to yield the desired compound (52 mg, 33%).

LC-MS (Method 2): m/z [M+H]⁺=412.2 (MW calc.=411.31); Rt=0.65 min.

¹H-NMR (DMSO-d₆, 400 MHz), δ (ppm)=10.39 (s, 1H), 8.59 (s, 2H), 8.15 (s, 1H), 7.97-7.60 (t, J=72.6 Hz, 1H), 6.95 (s, 1H), 6.88 (s, 1H), 3.94 (s, 3H), 3.77 (s, 3H).

### Example 45

### 5-[6-(Cyclopropyl-methoxy)-4-methoxy-pyridin-3-yl]-N-(2,6-difluoro-phenyl)-1-methyl-1H-pyrazole-3-carboxylic acid amide

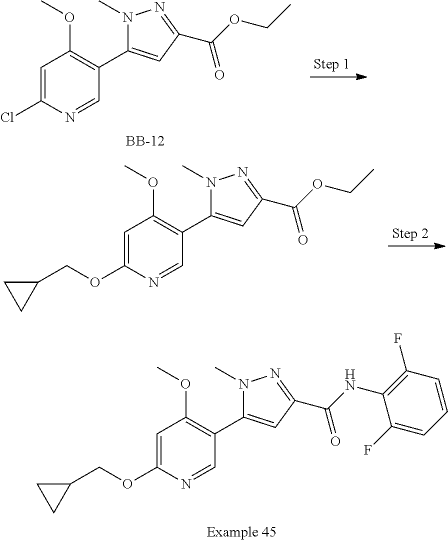

Step 1:

To a degassed suspension of BB-12 (600 mg, 2.03 mmol), cesium carbonate (1.65 mg, 5.07 mmol), XPHOS (56 mg, 126 μmol) and palladium acetate (42 mg, 213 μmol) in toluene is added cyclopropyl methanol (482 μL, 6.09 mmol) and the mixture was is heated under N₂ under in a microwave to 100° C. for 1 h. The mixture was extracted with EtOAC, the volatiles were removed under reduced pressure and the residue was purified by chromatography (Interchim® cartridge50SiHP/25 g, Cy/EtOAc) to yield the desired compound (50 mg, 7%).

LC-MS (Method 2): m/z [M+H]⁺=332.3 (MW calc.=331.37); Rt=0.85 min.

Step 2:

The title compound was prepared in analogy to step 2 of example 9 (28 mg, 46%).

LC-MS (Method 2): m/z [M+H]⁺=415.2 (MW calc. 414.41); Rt=0.74 min.

¹H NMR (600 MHz, DMSO-d6) δ=9.85 (s, 1H), 8.00 (s, 1H), 7.39 (tt, J=8.4, 6.2 Hz, 1H), 7.22-7.13 (m, 2H), 6.77 (s, 1H), 6.60 (s, 1H), 4.16 (d, J=7.2 Hz, 2H), 3.88 (s, 3H), 3.74 (s, 2H), 1.27 (dddd, J=11.9, 7.2, 5.3, 2.8 Hz, 1H), 0.67-0.47 (m, 2H), 0.35 (dt, J=6.1, 4.3 Hz, 2H).

### Example 46

### N-(2,6-Difluoro-phenyl)-5-[4-methoxy-6-(2,2,2-trifluoro-ethoxy)-pyridin-3-yl]-1-methyl-1H-pyrazole-3-carboxylic acid amide

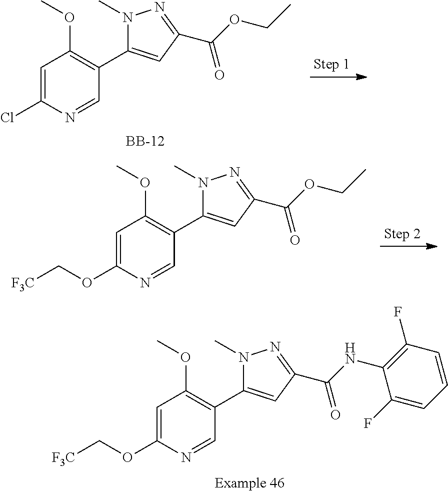

The title compound was prepared in analogy of example 45 in 2 steps starting from BB-12 (500 mg, 1.69 mmol) (28 mg, 2%).

LC-MS (Method 2): m/z [M+H]⁺=443.1 (MW calc. 442.34); Rt=0.74 min.

¹H NMR (600 MHz, DMSO-d6) δ=9.87 (s, 1H), 8.09 (d, J=1.0 Hz, 1H), 7.40 (ddd, J=14.6, 8.4, 6.2 Hz, 1H), 7.18 (t, J=8.0 Hz, 2H), 6.83-6.79 (m, 2H), 5.10-5.02 (m, 2H), 3.92 (d, J=1.0 Hz, 3H), 3.76 (d, J=1.0 Hz, 3H).

### Example 47

### 5-[4-(Difluoro-methoxy)-6-ethoxy-pyridin-3-yl]-N-(2,6-difluoro-phenyl)-1-methyl-1H-pyrazole-3-carboxylic acid amide

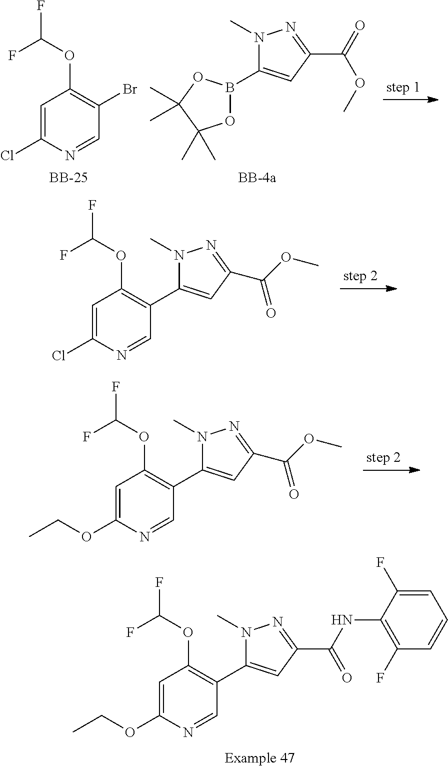

Step 1:

A mixture of BB-25 (550 mg, 2.13 mmol), BB-4a (510 mg, 1.91 mmol) and anhydrous LiOH (51 mg, 2.23 mmol) in DMF (10 mL) was degassed through purgin with Ar for 30 min. Bis(tri-tert-butylposphine)palladium (107 mg, 0.21 mmol) was added to the RM and heated to 90° C. for 2 h. The RM was diluted with water and extracted with EtOAc. The combined organic layers were washed with water and brine, were dried and the volatiles were removed under reduced pressure. The crude product which was purified by CC (SiO₂, EtOAc/Hex) to yield the desired compound (190 mg, 28%).

Step-2: A mixture of step 1 intermediate (190 mg, 0.60 mmol) and Cs₂CO₃ (487 mg, 1.50 mmol) in toluene (6 mL) was degassed through purging with Ar for 30 min. Pd(OAc)₂ (13 mg, 0.06 mmol), XPHOS (28 mg, 0.06 mmol) and EtOH (104 μl, 1.79 mmol) were added to the RM and heated in seal tube to 100° C. for 14 h. The RM was filtered and the volatiles were removed under reduced pressure to get a crude compound which was purified by CC (SiO₂, EtOAc/Hex) to yield the desired compound (90 mg, 46%).

LC-MS (Method 1): m/z [M+H]⁺=328.0 (MW calc.=327.28); Rₜ=3.18 min.

Step-3: To a solution of step 2 intermediate (90 mg, 275 μmol) and 2,6-difluoroaniline (71 mg, 550 μmol) in toluene (3 mL) was added Me₃Al (670 μl, 1.35 mmol) and the RM was heated at 110° C. for 1 h. The RM was cooled to RT and was treated with sat. NH₄Cl solution and was extracted with EtOAc. The combined organic layers were washed with water and brine, were dried and the volatiles were removed under reduced pressure to get a crude compound which was purified by CC (SiO₂, EtOAc) to yield the desired compound (53 mg, 45%).

LC-MS (Method 2): m/z [M+H]⁺=425.2 (MW calc.=424.35); Rₜ=0.73 min.

¹HNMR (DMSO-d₆, 400 MHz), δ (ppm)=9.93 (s, 1H), 8.26 (s, 1H), 7.72-7.36 (m, 2H), 7.18 (t, J=8.0 Hz, 2H), 6.84 (s, 1H), 6.80 (s, 1H), 4.40 (q, J=7.08 Hz, 2H), 3.78 (s, 3H), 1.35 (t, J=7.04 Hz, 3H).

### Example 48

### 5-[6-Cyclopropyl-4-(trifluoromethyl)-pyridin-3-yl]-N-(2,6-difluoro-phenyl)-1-methyl-1H-pyrazole-3-carboxylic acid

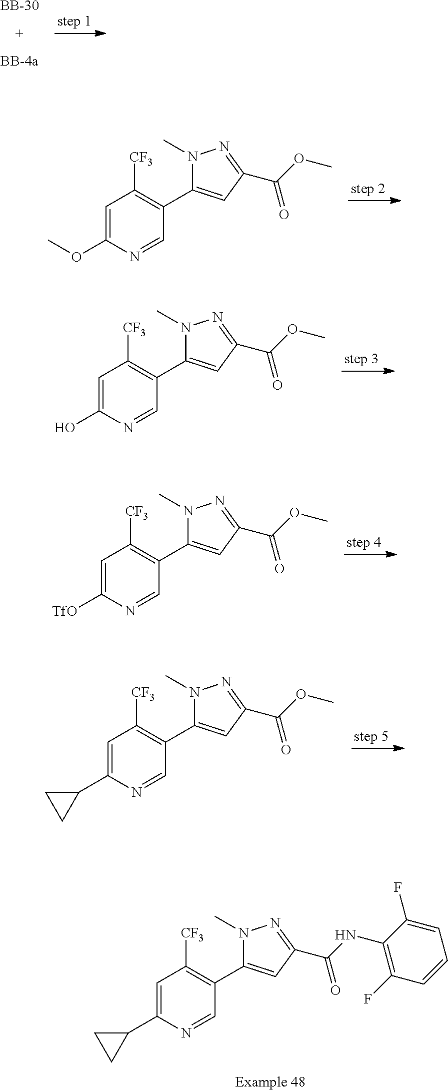

Step 1:

A mixture of 5-bromo-2-methoxy-4-trifluoromethyl-pyridine (BB-30, 1.7 g, 6.64 mmol), BB-4a (1.94 g, 7.30 mmol) and K₂CO₃ (2.74 g, 19.92 mmol) in dioxane (25 mL) and water (5 mL) was degassed with Ar for 30 min. Bis(tri-tert-butylposphine)palladium (337 mg, 0.66 mmol) was added and the RM heated to 100° C. for 14 h. The RM was concentrated under reduced pressure, diluted with EtOAc and washed with water and brine. The organic layer was dried and concentrated under reduced pressure. The crude product was purified by CC (SiO₂; 20% EtOAc/Hex) to yield the desired compound (1.3 g, 62%).

LC-MS (Method 4): m/z [M+H]⁺=316.2 (MW calc.=315.25); Rₜ=3.25 min.

Step 2:

To a suspension of NaI (6.6 g, 44.4 mmol) in CH₃CN (20 mL) at 0° C. was added TMS-Cl (5.6 ml, 44.4 mmol) and stirred for 10 min. A solution of the intermediate from step 1 (2.8 g, 8.8 mmol) in CH₃CN (20 mL) was added to the RM and stirred at RT for 2 h. The RM was diluted with water and extracted with EtOAc. The organic layer was washed with water and brine. The solvent was evaporated under reduced pressure to give the crude product which was purified by CC (SiO₂; basified with ammonia; 7% MeOH/CH₂Cl₂) to yield the desired compound (1.5 g, 56%).

LC-MS (Method 4): m/z [M+H]⁺=302.1 (MW calc.=301.22); Rₜ=2.44 min.

Step 3:

To a solution of the intermediate from step 2 (1.5 g, 4.98 mmol) in CH₂Cl₂ (30 mL) were added pyridine (0.81 ml, 9.96 mmol) and trifluoromethanesulfonic anhydride (1.26 ml, 7.47 mmol) at 0° C. and the RM was stirred at RT for 14 h. The RM was diluted with CH₂Cl₂ before being washed with water and brine. The organic layer was dried and concentrated under reduced pressure to give the crude product which was purified by CC (SiO₂; 30% EtOAc/Hex) to yield the desired compound (700 mg, 32%).

LC-MS (Method 4): m/z [M+H]⁺=433.8 (MW calc.=433.28); Rₜ=3.46 min.

Step 4:

A mixture of the intermediate from step 3 (700 mg, 1.61 mmol), potassium cyclopropyltrifluoroborate (358 mg, 2.42 mmol) and K₂CO₃ (444 mg, 3.22 mmol) in toluene (10 mL) and water (2 mL) was degassed with Ar for 30 min. Pd(OAc)₂ (36 mg, 0.16 mmol) and di-(1-adamantyl)-n-butylphosphine (57 mg, 0.16 mmol) were added to the RM and heated at 100° C. for 14 h. The RM was concentrated under reduced pressure, diluted with EtOAc and washed with water and brine. The organic layer was dried and concentrated under reduced pressure to give the crude product which was purified by CC (SiO₂; 20% EtOAc/Hex) to yield the desired compound (320 mg, 61%).

LC-MS (Method 5): m/z [M+H]⁺=326.2 (MW calc.=325.29); Rₜ=3.37 min.

Step 5:

The title compound was prepared in analogy to step 2 of example 9 (48 mg, 38%).

LC-MS (Method 2): m/z [M+H]⁺=423.1 (MW calc.=422.35); Rₜ=0.79 min.

¹H NMR (DMSO-d₆, 400 MHz), δ (ppm)=9.97 (s, 1H), 8.68 (s, 1H), 7.93 (s, 1H), 7.41-7.35 (m, 1H), 7.18 (t, 2H, J=8.4 Hz), 6.86 (s, 1H), 3.71 (s, 3H), 2.44-2.40 (m, 1H), 1.13-1.08 (m, 4H).

### Example 49

### N-(2,6-Difluoro-phenyl)-5-[6-ethoxy-4-(oxetan-3-yloxy)-pyridin-3-yl]-1-methyl-1H-pyrazole-3-carboxylic acid amide

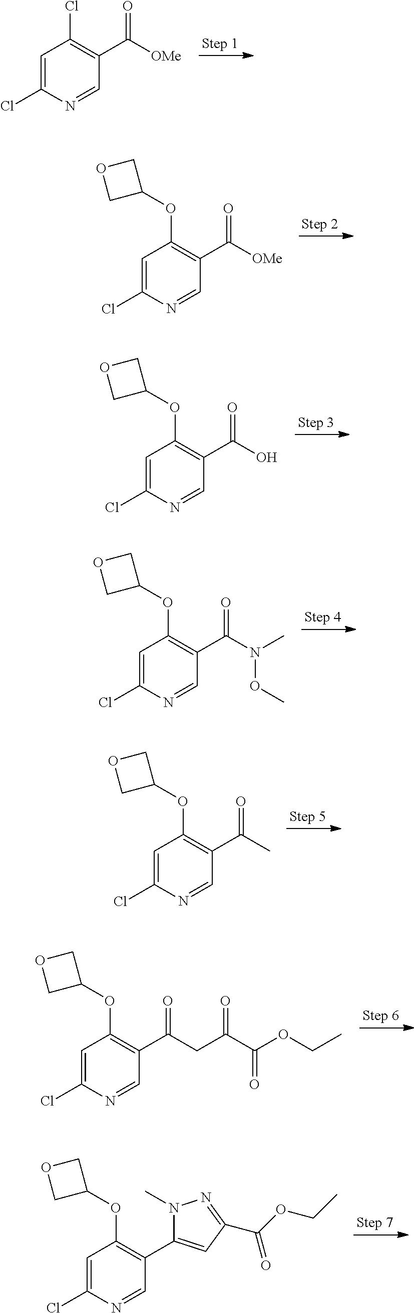

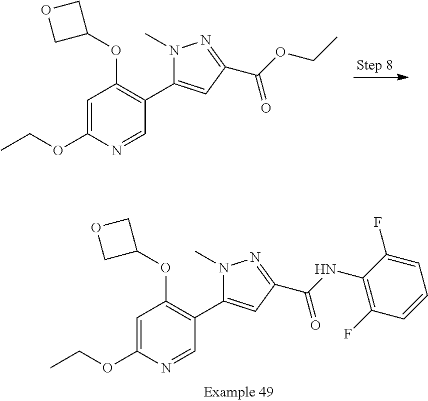

Step 1:

To a solution of oxetan-3-ol (3.07 mL, 48.54 mmol) in THF (150 mL) was added NaH (1.94 g, 48.54 mmol, 60% in mineral oil) at RT and the resulting mixture heated to reflux for 30 min. The RM was cooled to 0° C., 4,6-dichloro-nicotinic acid methyl ester (10.0 g, 48.54 mmol) was added and stirring continued at RT for 3 h. The RM was quenched with ice cold water (200 mL), extracted with EtOAc and dried. The solvent was evaporated under reduced pressure to give the crude product which was purified by CC (SiO₂; 20% EtOAc/Hex) to afford 6-chloro-4-(oxetan-3-yloxy)-nicotinic acid methyl ester (7.2 g, 61%).

LC-MS (Method 4): m/z [M+H]⁺=244.4 (MW calc.=243.64); Rₜ=2.57 min.

Step 2:

To a solution of 6-chloro-4-(oxetan-3-yloxy)-nicotinic acid methyl ester (7.2 g, 29.62 mmol) in a mixture of THF:H₂O (1:1, 150 mL) was added portion-wise LiOH.H₂O (3.11 g, 74.07 mmol) at 0° C. and the RM was stirred at 0° C. for 30 min. The RM was concentrated under reduced pressure and diluted with water, acidified with sat.NaHSO₄ solution up to pH˜3 and extracted with EtOAc. The organic layer was dried and concentrated under reduced pressure to give 6-chloro-4-(oxetan-3-yloxy)-nicotinic acid (6.2 g, 91%).

LC-MS (Method 4): m/z [M+H]⁺=230.1 (MW calc.=229.62); Rₜ=0.70 min.

Step 3:

To a cooled (0° C.) solution of 6-chloro-4-(oxetan-3-yloxy)-nicotinic acid (6.2 g, 27.07 mmol) in CH₂Cl₂ (200 mL) were added DIPEA (14.12 ml 81.21 mmol) and HATU (10.29 g, 27.07 mmol) and the RM was stirred for 15 min. Subsequently O,N-dimethyl-hydroxylamine hydrochloride (2.64 g, 27.07 mmol) was added to the RM and stirring continued at RT for 16 h. The RM was diluted with CH₂Cl₂ (200 mL) and washed with sat. NH₄Cl solution, sat. NaHCO₃ solution, water and brine and dried. The solvent was evaporated under reduced pressure to give the crude product which was purified by CC (SiO₂; 40% EtOAc/Hex) to yield 6-chloro-N-methoxy-N-methyl-4-(oxetan-3-yloxy)-nicotinamide (6.6 g, 89%).

LC-MS (Method 4): m/z [M+H]⁺=273.0 (MW calc.=272.68); Rₜ=2.03 min.

Step 4:

To a solution of 6-chloro-N-methoxy-N-methyl-4-(oxetan-3-yloxy)-nicotinamide (7.1 g, 26.10 mmol) in THF (140 mL) was added a 3M solution of methylmagnesium bromide in Et₂O (13.05 mL, 39.15 mmol) at −10° C. and the RM was stirred for 4 h. The RM was quenched with sat.NH₄Cl solution and extracted with EtOAc. The organic layer was dried and concentrated under reduced pressure to give the crude product which was purified by CC (SiO₂; 30% EtOAc/Hex) to afford 1-[6-chloro-4-(oxetan-3-yloxy)-pyridin-3-yl]-ethanone (4.12 g, 69%).

LC-MS (Method 4): m/z [M+H]⁺=228.1 (MW calc.=227.64); Rₜ=2.35 min.

Step 5:

A 1M solution of LHMDS in THF (27.22 mL, 27.22 mmol) was added drop-wise to a mixture of 1-[6-chloro-4-(oxetan-3-yloxy)-pyridin-3-yl]-ethanone (4.12 g, 18.14 mmol) and diethyl oxalate (2.48 mL, 18.15 mmol) in THF (100 mL) at 0° C. and the RM was stirred at RT for 2 h. The RM was quenched with sat.NH₄Cl solution and extracted with EtOAc. The organic layer was dried and concentrated to give the desired product (5.0 g, 84%) which was used in the next step without further purification.

LC-MS (Method 4): m/z [M+H]⁺=328.0 (MW calc.=327.72); Rₜ=1.99 min.

Step 6:

A mixture of the intermediate from step 5 (2.5 g, 7.64 mmol) and methylhydrazine (0.4 mL, 7.64 mmol) in EtOH (80 mL) was heated at reflux for 16 h. The RM was concentrated, diluted with EtOAc and washed with water, sat.NH₄Cl and brine. The organic layer was dried and concentrated under reduced pressure to give the crude compound which was purified by CC (SiO₂; 40% EtOAc/Hex) to yield 5-[6-chloro-4-(oxetan-3-yloxy)-pyridin-3-yl]-1-methyl-1H-pyrazole-3-carboxylic acid ethyl ester (500 mg, 19%).

LC-MS (Method 4): m/z [M+H]⁺=338.4 (MW calc.=337.76); Rₜ=3.14 min.

Step 7:

A mixture of the intermediate from step 6 (500 mg, 1.48 mmol) and Cs₂CO₃ (1.2 g, 3.7 mmol) in toluene (22 mL) was degassed with Ar for 30 min. Pd(OAc)₂ (33 mg, 0.148 mmol), X-phos (35 mg, 0.074 mmol) and EtOH (0.26 ml, 4.44 mmol) were added to the RM and heated in a sealed tube at 110° C. for 16 h. The RM was diluted with EtOAc and washed with water and brine. The organic layer was dried and concentrated under reduced pressure to give the crude product which was purified by CC (SiO₂; 15% acetone/Hex) to yield the desired compound (400 mg, 77%).

LC-MS (Method 4): m/z [M+H]⁺=348.2 (MW calc.=347.37); Rₜ=2.97 min.

Step 8:

The title compound was prepared in analogy to example 47 (80 mg, 64%).

LC-MS (Method 2): m/z [M+H]⁺=431.2 (MW calc.=430.40); Rₜ=0.68 min.

¹H NMR (DMSO-d₆, 400 MHz), δ (ppm)=9.91 (s, 1H), 8.08 (s, 1H), 7.42 (m, 1H), 7.18 (t, 2H, J=7.6 Hz), 6.82 (s, 1H), 6.21 (s, 1H), 5.46-5.41 (m, 1H), 4.94 (t, 2H, 6.8 Hz), 4.53-4.50 (m, 2H), 4.34 (q, 2H, J=6.8 Hz), 3.82 (s, 3H), 1.33 (t, 3H, J=6.8 Hz).

### Example 50

### 5-(6-Chloro-4-methoxy-pyridin-3-yl)-N-(2,6-difluoro-phenyl)-1-methyl-1H-pyrazole-3-carboxylic acid amide

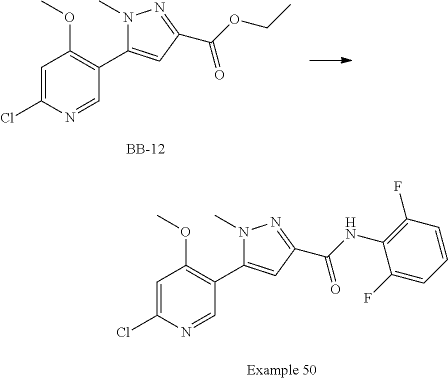 [SMILES: CCOC(=O)c1cc(-c2cnc(Cl)cc2OC)n(C)n1 ; "BB-12" ; COc1cc(Cl)ncc1-c1cc(C(=O)Nc2c(F)cccc2F)nn1C]

A degassed solution of 2,6-difluroaniline (3.06 mL, 23.7 mmol) in dry toluene (31 mL) was treated with AlMe₃ (2M in toluene, 5.07 mL, 10.1 mmol) at 0° C. and the mixture was stirred at 0° C. for 30 min. BB-12 (1.00 g, 3.38 mmol) was added as a solution in toluene and the mixture was stirred at 120° C. for 1 h. The mixture was chilled an water and NaOH were carefully added. The layers were separated and the aqueous layer was extracted with EtOAc. The combined organic layers were dried and the volatiles were removed under reduced pressure. The residue was purified through chromatography (Interchim® cartridge50SiHP/80 g, Cy/EtOAc) to yield the desired compound (700 mg, 55%).

LC-MS (Method 2): m/z [M+H]⁺=379.1 (MW calc.=378.77); Rt=0.66 min.

¹H-NMR (DMSO-d₆, 600 MHz), δ (ppm)=9.89 (s, 1H), 8.29 (s, 1H), 7.41 (s, 1H), 7.42 7.36 (m, 1H), 7.19 (t, J=8.0 Hz, 2H), 6.88 (s, 1H), 3.97 (s, 3H), 3.78 (s, 3H).

### Example 51

### 5-(6-Chloro-4-methoxy-pyridin-3-yl)-N-(2,6-difluoro-phenyl)-1-methyl-1H-pyrazole-3-carboxylic acid amide

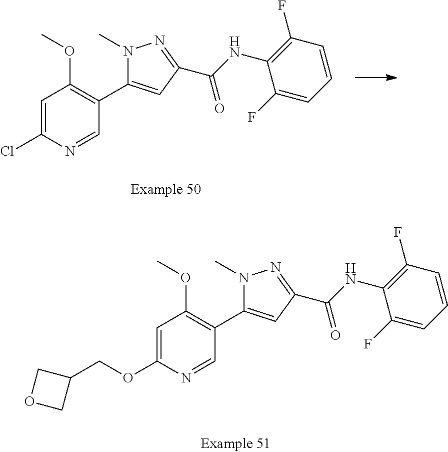 [SMILES: COc1cc(Cl)ncc1-c1cc(C(=O)Nc2c(F)cccc2F)nn1C ; COc1cc(OCC2COC2)ncc1-c1cc(C(=O)Nc2c(F)cccc2F)nn1C]

To a degassed suspension of example 50 (250 mg, 660 μmol), Cs₂CO₃ (538 mg, 1.65 mmol), X-phos (19 mg, 41 μmol) and Pd(OAc)₂ (14 mg, 69 μmol) in toluene is added oxetan-3-yl-methanol (116 μL, 1.32 mmol) and the mixture was heated under N₂ under in a microwave to 100° C. overnight. The mixture was extracted with EtOAC, the volatiles were removed under reduced pressure and the residue was purified by chromatography (Interchim® cartridge50SiHP/12 g, Cy/EtOAc) to yield the desired compound (37 mg, 13%).

LC-MS (Method 2): m/z [M+H]⁺=431.1 (MW calc.=430.41); Rt=0.66 min.

¹H-NMR (DMSO-d₆, 400 MHz), δ (ppm)=9.83 (s, 1H), 8.03 (s, 1H), 7.42-7.35 (m, 1H), 7.21-7.14 (m, 2H), 6.77 (s, 1H), 6.61 (s, 1H), 4.74-4.70 (m, 2H), 4.54 (d, J=6.8 Hz, 2H), 4.45 (t, J=6.4H

### Example 52

### N-(3,5-Difluoro-pyridin-4-yl)-5-(6-isopropoxy-4-methyl-pyridin-3-yl)-1-methyl-1H-pyrazole-3-carboxylic acid amide

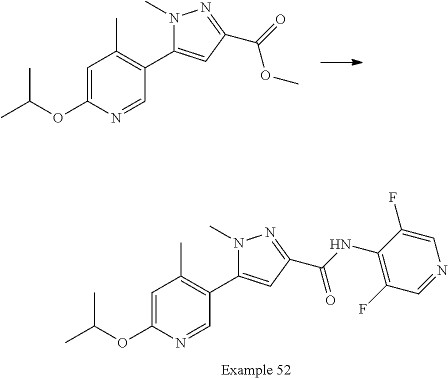

The title compound was prepared in analogy of example 45 starting from intermediate 1 of example 13 (220 mg, 725 μmol) (47 mg, 17%).

LC-MS (Method 2): m/z [M+H]⁺=388.1 (MW calc. 387.39); Rt=0.74 min.

¹H-NMR (400 MHz, DMSO-d6) δ=10.37 (s, 1H), 8.59 (s, 2H), 8.10 (s, 1H), 6.87 (s, 1H), 6.79 (s, 1H), 5.33-5.26 (m, 1H), 3.75 (s, 3H), 2.14 (s, 3H), 1.32 (d, J=6.2 Hz, 6H) ppm.

### Example 53

### 5-(6-Cyclopropyl-4-methoxy-pyridin-3-yl)-N-(3,5-difluoro-pyridin-4-yl)-1-methyl-1H-pyrazole-3-carboxylic acid amide

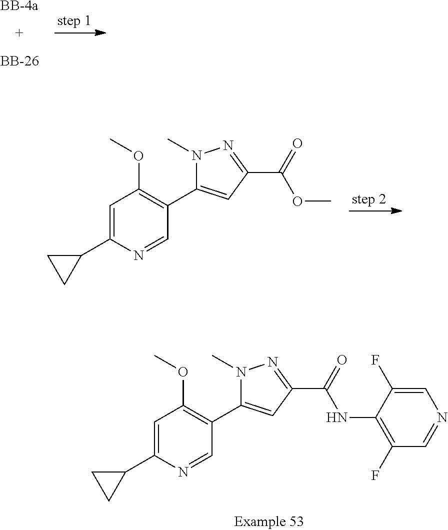 [SMILES: "BB-4a" ; "BB-26" ; COC(=O)c1cc(-c2cnc(C3CC3)cc2OC)n(C)n1 ; COc1cc(C2CC2)ncc1-c1cc(C(=O)Nc2c(F)cncc2F)nn1C]

The title compound was synthesized in analogy to example 42 in 2 steps starting from BB-26 (200 mg, 877 μmol) and BB-4a (491 mg, 1.75 mmol) (10 mg, 8% over 2 steps).

LC-MS (Method 2): m/z [M+H]⁺=386.1 (MW calc.=385.37); Rt=0.40 min.

¹H-NMR (600 MHz, CDCl₃) δ=8.51 (s, 1H), 8.42 (d, J=2.6 Hz, 2H), 8.23 (d, J=2.9 Hz, 1H), 6.92 (d, J=2.6 Hz, 1H), 6.83 (d, J=2.6 Hz, 1H), 3.80 (s, 3H), 2.12-2.03 (m, 1H), 1.17-1.04 (m, 4H) ppm.

### Example 54

### 5-(6-Cyclopropyl-4-methoxy-pyridin-3-yl)-N-(2,6-difluoro-phenyl)-1-methyl-1H-pyrazole-3-carboxylic acid amide

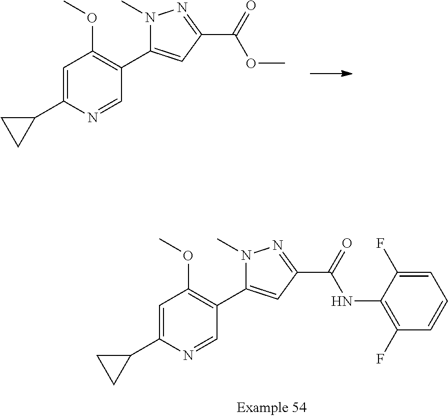 [SMILES: COC(=O)c1cc(-c2cnc(C3CC3)cc2OC)n(C)n1 ; COc1cc(C2CC2)ncc1-c1cc(C(=O)Nc2c(F)cccc2F)nn1C]

The title compound was synthesized in analogy to example 42 starting from step 1 intermediate of example 53 (300 mg, 996 μmol) (15 mg, 4%).

LC-MS (Method 2): m/z [M+H]⁺=385.1 (MW calc.=384.38); Rt=0.47 min.

¹H NMR (600 MHz, CDCl₃) δ=8.27 (s, 1H), 7.25-7.20 (m, 1H), 7.03-6.98 (m, 2H), 6.91 (s, 1H), 6.81 (s, 1H), 3.92 (s, 1H), 3.80 (s, 3H), 2.08 (ddd, J=8.2, 4.8, 3.4 Hz, 1H), 1.16-1.12 (m, 2H), 1.09-1.04 (m, 2H) ppm.

### Example 55

### 5-[6-(Cyclobutyl-methoxy)-4-methoxy-pyridin-3-yl]-N-(2,6-difluoro-phenyl)-1-methyl-1H-pyrazole-3-carboxylic acid amide

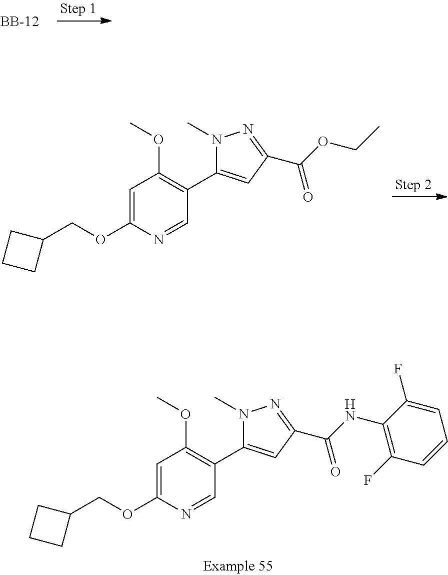 [SMILES: "BB-12" ; CCOC(=O)c1cc(-c2cnc(OCC3CCC3)cc2OC)n(C)n1 ; COc1cc(OCC2CCC2)ncc1-c1cc(C(=O)Nc2c(F)cccc2F)nn1C]

The title compound was prepared in analogy of example 45 in 2 steps starting from BB-12 (500 mg, 1.69 mmol) (85 mg, 13% over 2 steps).

LC-MS (Method 2): m/z [M+H]⁺=429.2 (MW calc. 428.44); Rt=0.86 min.

¹H-NMR (600 MHz, DMSO-d6) δ=9.84 (s, 1H), 8.01 (s, 1H), 7.39 (tt, J=8.5, 6.2 Hz, 1H), 7.18 (t, J=8.0 Hz, 2H), 6.78 (s, 1H), 6.58 (s, 1H), 4.30 (d, J=6.8 Hz, 2H), 3.88 (s, 3H), 3.75 (s, 3H), 2.79-2.71 (m, 1H), 2.13-2.05 (m, 2H), 1.99-1.79 (m, 4H) ppm.

### Example 56

### 5-(4-Chloro-6-methoxy-pyridin-3-yl)-N-(2,6-difluoro-phenyl)-1-methyl-1H-pyrazole-3-carboxylic acid amide

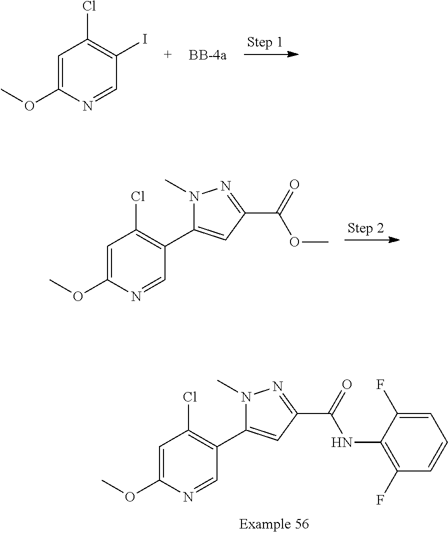

The title compound was synthesized in analogy to example 42 in 2 steps starting from commercially available 4-Chloro-5-jodo-2-methoxypyridine (400 mg, 1.48 mmol) and BB-4a (831 mg, 2.97 mmol) (206 mg, 36% over 2 steps).

LC-MS (Method 2): m/z [M+H]⁺=379.1 (MW calc.=378.77); Rt=0.73 min.

¹H NMR (600 MHz, CDCl₃) δ=9.93 (s, 1H), 8.35 (s, 1H), 7.40 (tt, J=8.4, 6.2 Hz, 1H), 7.26 (s, 1H), 7.19 (t, J=8.1 Hz, 2H), 6.90 (s, 1H), 3.96 (s, 3H), 3.79 (s, 3H) ppm.

### Example 57

### 4-Chloro-N-(2,6-difluoro-phenyl)-5-(4,6-dimethoxy-pyridin-3-yl)-1-methyl-1H-pyrazole-3-carboxylic acid amide

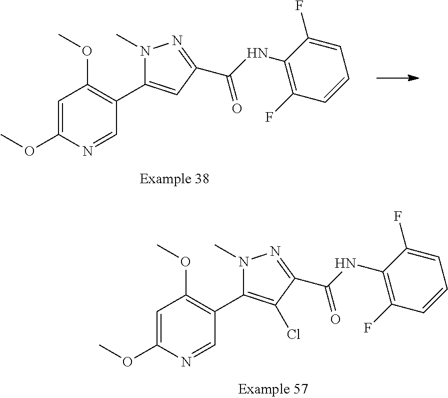 [SMILES: COc1cc(OC)c(-c2cc(C(=O)Nc3c(F)cccc3F)nn2C)cn1 ; COc1cc(OC)c(-c2c(Cl)c(C(=O)Nc3c(F)cccc3F)nn2C)cn1]

A solution of example compound 38 (99 mg, 267 μmol) in CH₃CN (0.7 mL) was treated with N-chlorosuccinimide (36 mg, 267 μmol) and the mixture was stirred at 80° C. overnight. The volatiles were removed under reduced pressure and the residue was purified by chromatography (Interchim® cartridge50SiHP/12 g, Cy/EtOAc) to give the desired compound (33 mg, 30%).

LC-MS (Method 2): m/z [M+H]⁺=409.1 (MW calc.=408.79); Rt=0.71 min.

¹H-NMR (600 MHz, CDCl₃) δ=9.96 (s, 1H), 8.05 (s, 1H), 7.40 (tt, J=8.4, 6.2 Hz, 1H), 7.19 (t, J=8.0 Hz, 2H), 6.63 (s, 1H), 3.93 (s, 3H), 3.88 (s, 3H), 3.75 (s, 3H) ppm.

### Example 58

### N-(2,6-Difluoro-phenyl)-5-(6-methoxy-4-methyl-pyridin-3-yl)-1-methyl-1H-pyrazole-3-carboxylic acid amide

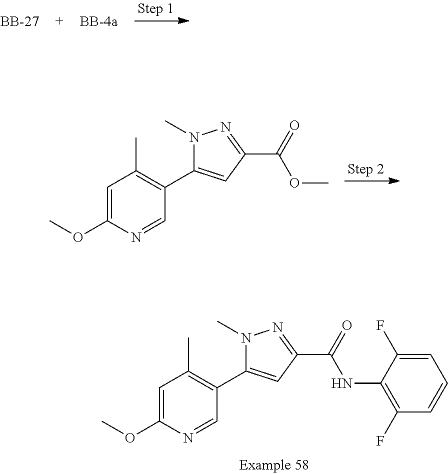

The title compound was synthesized in analogy to example 42 in 2 steps starting from BB-27 (770 mg, 3.81 mmol) and BB-4a (2.13 g, 2.97 mmol) (14% over 2 steps).

LC-MS (Method 2): m/z [M+H]⁺=359.1 (MW calc.=358.3); Rt=0.68 min.

¹H-NMR (400 MHz, CDCl₃) δ=9.88 (s, 1H), 8.12 (s, 1H), 7.42-7.36 (m, 1H), 7.21-7.14 (m, 2H), 6.88 (s, 1H), 6.82 (s, 1H), 3.90 (s, 3H), 3.74 (s, 3H), 2.16 (s, 3H) ppm.

### Example 59

### N-(2,6-Difluoro-phenyl)-5-[6-(methoxymethyl)-4-methyl-pyridin-3-yl]-1-methyl-1H-pyrazole-3-carboxylic acid amide

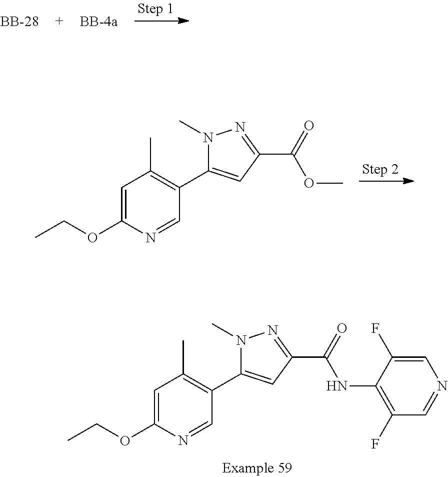

The title compound was synthesized in analogy to example 42 in 2 steps starting from BB-28 (99 mg, 463 μmol) and BB-4a (259 mg, 926 μmol) (106 mg, 63% over 2 steps).

LC-MS (Method 2): m/z [M+H]⁺=373.2 (MW calc.=372.4); Rt=0.57 min.

¹H NMR (400 MHz, CDCl₃) δ=9.93 (d, J=2.8 Hz, 1H), 8.45 (s, 1H), 7.49-7.45 (m, 1H), 7.44-7.37 (m, 1H), 7.19 (td, J=8.0, 2.7 Hz, 2H), 6.92-6.87 (m, 1H), 4.55 (d, J=2.5 Hz, 2H), 3.78-3.74 (m, 3H), 2.25 (s, 3H) ppm.

### Example 60

### N-(2,6-Difluoro-phenyl)-1-methyl-5-[4-methyl-6-(trifluoromethyl)-pyridin-3-yl]-1H-pyrazole-3-carboxylic acid amide

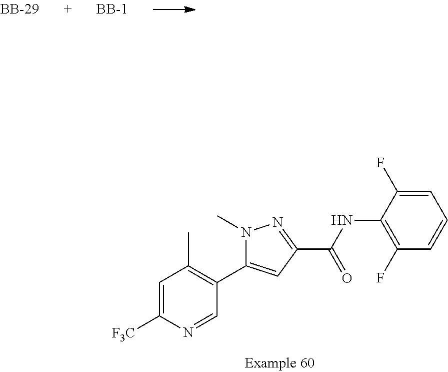 [SMILES: "BB-29" ; "BB-1" ; Cc1cc(C(F)(F)F)ncc1-c1cc(C(=O)Nc2c(F)cccc2F)nn1C]

A mixture of BB-28 (136 mg, 475 μmol), BB-1 (150 mg, 475 mmol), bis(tri-tert-butyl-phosphine)palladium(0) (16 mg, 33 μmol) and LiOH (12 mg, 541 μmol) in dry DMF (2 mL) was stirred N₂ at 100° C. for 1 h. The volatiles were removed under reduced pressure and the residue was purified by preparative HPLC to yield the desired compound (32 mg, 17%).

LC-MS (Method 2): m/z [M+H]⁺=397.1 (MW calc.=396.3); Rt=0.72 min.

¹H-NMR (400 MHz, CDCl₃) δ=9.95 (s, 1H), 8.74 (s, 1H), 8.03 (s, 1H), 7.42-7.36 (m, 1H), 7.22-7.15 (m, 2H), 6.99 (s, 1H), 3.79 (s, 3H), 2.36 (s, 3H) ppm.

### Example 61

### 5-(6-Cyclopropyloxy-4-methoxy-pyridin-3-yl)-N-(2,6-difluoro-phenyl)-1-methyl-1H-pyrazole-3-carboxylic acid amide

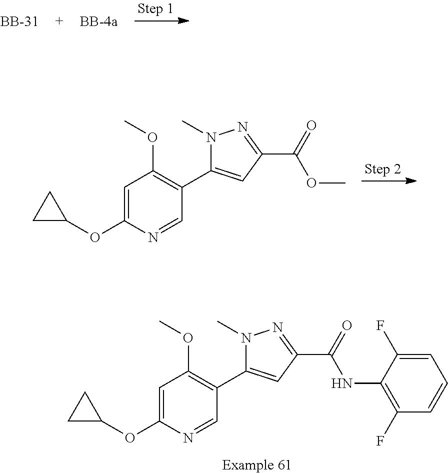 [SMILES: "BB-31" ; "BB-4a" ; COC(=O)c1cc(-c2cnc(OC3CC3)cc2OC)n(C)n1 ; COc1cc(OC2CC2)ncc1-c1cc(C(=O)Nc2c(F)cccc2F)nn1C]

Step 1:

A mixture of BB-31 (410 mg, 1.68 mmol), BB-4a (581 mg, 2.18 mmol) and potassium fluoride (292 mg, 5.04 mmol) in dioxane (20 mL) was degassed with Ar for 30 min. Bis(tri-tert-butylphosphine)palladium (429 mg, 0.84 mmol) was added to RM and heated at 100° C. for 4 h. The RM was concentrated to give the crude product which was purified by CC (SiO₂; 30% EtOAc/Hex) to yield the desired compound (200 mg, 39%).

LC-MS (Method 4): m/z [M+H]⁺=304.2 (MW calc. 303.31); Rₜ=2.97 min.

Step 2:

The title compound was prepared in analogy to example 39 (55 mg, 23%).

LC-MS (Method 2): m/z [M+H]⁺=401.1 (MW calc. 400.38); Rₜ=0.70 min.

¹H NMR (DMSO, 400 MHz), δ (ppm)=9.87 (s, 1H), 8.06 (s, 1H), 7.4-7.3 (m, 1H), 7.18 (t, 2H, J=8.0 Hz), 6.77 (s, 1H), 6.61 (s, 1H), 4.31-4.28 (m, 1H), 3.87 (s, 3H), 3.74 (s, 3H), 0.80-0.70 (m, 4H).

### Example 62

### N-(2,6-Difluoro-phenyl)-5-[4-methoxy-6-(oxetan-3-yloxy)-pyridin-3-yl]-1-methyl-1H-pyrazole-3-carboxylic acid amide

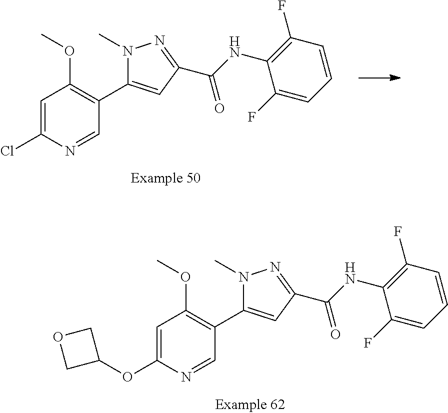 [SMILES: COc1cc(Cl)ncc1-c1cc(C(=O)Nc2c(F)cccc2F)nn1C ; COc1cc(OC2COC2)ncc1-c1cc(C(=O)Nc2c(F)cccc2F)nn1C]

To a solution of oxetan-3-ol (47 mg, 0.63 mmol) in NMP (5 mL) was added NaH (64 mg, 1.59 mmol, 60% in mineral oil) and stirred for 15 min. Example 50 (200 mg, 0.53 mmol) was added to the RM and heated at 100° C. for 16 h. The RM was cooled, diluted EtOAc and washed with water and brine. The organic layer was dried and concentrated under reduced pressure to give the crude product which was purified by CC (SiO₂; 40% EtOAc/Hex) and then by prep HPLC to yield the title compound.

LC-MS (Method 2): m/z [M+H]⁺=417.1 (MW calc.=416.38); Rₜ=0.64 min.

¹H NMR (DMSO-d₆, 400 MHz), δ (ppm)=9.85 (s, 1H), 7.99 (s, 1H), 7.42-7.36 (m, 1H), 7.17 (t, 2H, J=7.96 Hz), 6.76 (s, 1H), 6.70 (s, 1H), 5.65-5.60 (m, 1H), 4.91 (t, 2H, J=6.74 Hz), 4.61-4.57 (m, 2H), 3.89 (s, 3H), 3.73 (s, 3H).

### Example 63

### 5-[6-Cyclopropyl-4-(trifluoromethyl)-pyridin-3-yl]-N-(3,5-difluoro-pyridin-4-yl)-1-methyl-1H-pyrazole-3-carboxylic acid amide

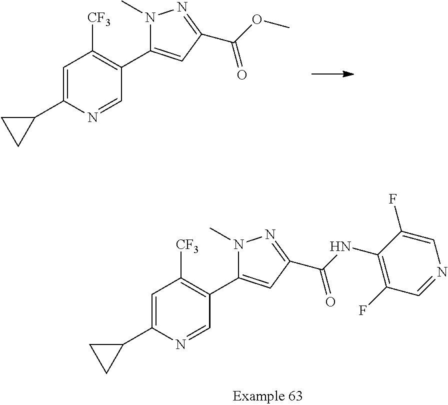 [SMILES: COC(=O)c1cc(-c2cnc(C3CC3)cc2C(F)(F)F)n(C)n1 ; Cn1nc(C(=O)Nc2c(F)cncc2F)cc1-c1cnc(C2CC2)cc1C(F)(F)F]

The title compound was prepared in analogy to example 48 (50 mg, 21%).

LC-MS (Method 2): m/z [M+H]⁺=424.1 (MW calc.=423.34); Rₜ=0.73 min.

¹H NMR (DMSO-d₆, 400 MHz), δ (ppm)=10.48 (s, 1H), 8.68 (s, 1H), 8.60 (s, 2H), 7.94 (s, 1H), 6.92 (s, 1H), 3.72 (s, 3H), 2.45-2.42 (m, 1H), 1.13-1.08 (m, 4H).

### Example 64

### N-(2,6-Difluoro-phenyl)-5-(4,6-dimethoxy-pyridin-3-yl)-1,4-dimethyl-1H-pyrazole-3-carboxylic acid amide

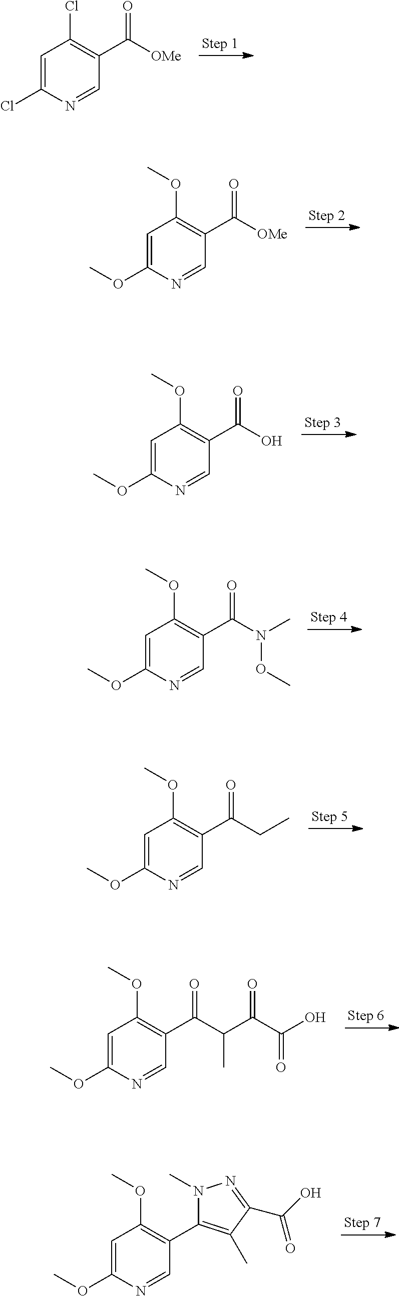

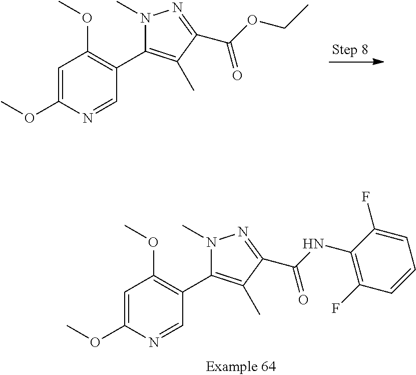

Step 1:

To a solution of sodium methoxide (10.5 g, 195 mmol.) in MeOH (250 mL) was added 4,6-dichloro-nicotinic acid methyl ester (10.0 g, 48.9 mmol) at 0° C. and the resulting RM was heated at reflux for 16 h. The RM was concentrated under reduced pressure, diluted with water and extracted with EtOAc. The organic layer was dried and concentrated under reduced pressure to give 4,6-dimethoxy-nicotinic acid methyl ester (6.0 g, 62%).

LC-MS (Method 4): m/z [M+H]⁺=198.0 (MW calc. 197.19); Rₜ=2.65 min.

Step 2:

To a cooled solution of 4,6-dimethoxy-nicotinic acid methyl ester (6.0 g, 30.45 mmol) in a mixture of MeOH:THF:H₂O (150 mL, 1:1:1 by volume) was added LiOH.H₂O (3.84 g, 91.37 mmol) at 0° C. and the resulting RM was stirred at RT for 3 h. The RM was concentrated under reduced pressure, diluted with water, acidified with sat.NaHSO₄ up to pH˜3 and extracted with 10% MeOH in CH₂Cl₂. The organic layer was dried and concentrated under reduced pressure to give 4,6-dimethoxy-nicotinic acid (5.2 g, 93%).

LC-MS (Method 4): m/z [M+H]⁺=183.9 (MW calc. 183.16); Rₜ=0.66 min.

Step 3:

To a solution of 4,6-dimethoxy-nicotinic acid (5.2 g, 28.41 mmol) in CH₂Cl₂ (150 mL) were added DIPEA (14.82 ml, 85.23 mmol) and HATU (10.8 g, 28.41 mmol) at 0° C. and stirred at 0° C. for 15 min. O,N-dimethyl-hydroxylamine hydrochloride (2.77 g, 28.41 mmol) was added to the RM at 0° C. and the resulting RM was stirred at RT for 16 h. The RM was diluted with CH₂Cl₂ and washed with water, sat. NH₄Cl, sat. NaHCO₃ and brine. The organic layer was dried and concentrated under reduced pressure to give the crude product which was purified by CC (SiO₂; 40% EtOAc/Hex) to yield 4,6,N-trimethoxy-N-methyl-nicotinamide (5.92 g, 92%).

LC-MS (Method 4): m/z [M+H]⁺=227.1 (MW calc. 226.23); Rₜ=2.41 min.

Step 4:

To a solution of 4,6,N-trimethoxy-N-methyl-nicotinamide (5.92 g, 26.19 mmol) in THF (150 mL) was added drop-wise ethyl magnesium bromide (10.48 ml, 31.43 mmol, 3M in Et₂O) at −10° C. and the resulting RM was stirred at −10° C. for 2 h. The RM was quenched with sat.NH₄Cl solution and extracted with EtOAc. The organic layer was dried and concentrated under reduced pressure to give the crude product which was purified by CC (SiO₂; 6% EtOAc/Hex) to afford 1-(4,6-dimethoxy-pyridin-3-yl)-propan-1-one (3.5 g, 68%).

LC-MS (Method 4): m/z [M+H]⁺=196.3 (MW calc. 195.22); Rₜ=3.04 min.

Step 5:

To a solution of 1-(4,6-dimethoxy-pyridin-3-yl)-propan-1-one (3.75 g, 19.23 mmol) in THF (160 mL) was added drop-wise LHMDS (48.07 mL, 48.07 mmol) at −20° C. and the resulting RM was stirred at −20° C. for 3 h. A solution of Imidazol-1-yl-oxo-acetic acid ethyl ester (6.46 g, 38.46 mmol) in THF (30 mL) was added to the RM at −20° C. and the resulting RM was stirred at RT for 16 h. The RM was diluted with EtOAc and sat.NH₄Cl and the layers were separated. The organic layer was dried and concentrated under reduced pressure to give 4-(4,6-dimethoxy-pyridin-3-yl)-3-methyl-2,4-dioxo-butyric acid (5.1 g) which was used in the next step without purification.

LC-MS (Method 4): m/z [M+H]⁺=268.2 (MW calc. 267.23); Rₜ=1.56 min.

Step 6:

To a crude solution of 4-(4,6-dimethoxy-pyridin-3-yl)-3-methyl-2,4-dioxo-butyric acid (5.1 g, 19.1 mmol) in EtOH (190 mL) was added methyl hydrazine (1.0 mL, 19.1 mmol) at RT and the resulting RM was heated at reflux for 16 h. The RM was concentrated under reduced pressure, diluted with water and washed with CH₂Cl₂. The aqueous layer was acidified with sat.NaHSO₄ solution up to pH˜2-3 and extracted with CH₂Cl₂. The organic layer was dried and concentrated under reduced pressure to a mixture of desired 5-(4,6-dimethoxy-pyridin-3-yl)-1,4-dimethyl-1H-pyrazole-3-carboxylic acid and undesired 5-(4,6-dimethoxy-pyridin-3-yl)-2,4-dimethyl-2H-pyrazole-3-carboxylic acid (2.15 g) which was used in the next step without purification.

LC-MS (Method 4): m/z [M+H]⁺=278.3 (MW calc. 277.28); Rₜ=1.61 min and 1.46 min.

Step 7:

To the intermediate from step 6 (2.15 g, 7.76 mmol) in acetone (80 mL) were added K₂CO₃ (5.35 g, 38.8 mmol) and ethyl iodide (1.88 mL, 23.28 mmol) at 0° C. and the resulting RM was stirred at RT for 16 h. The RM was concentrated under reduced pressure, diluted with water and extracted with EtOAc. The organic layer was washed with sat. Na₂S₂O₃ solution. The organic layer was dried and concentrated under reduced pressure to give the crude product which was purified by CC (SiO₂; 8% Acetone/Hex) to yield 5-(4,6-dimethoxy-pyridin-3-yl)-1,4-dimethyl-1H-pyrazole-3-carboxylic acid ethyl ester (800 mg, 13%).

LC-MS (Method 4): m/z [M+H]⁺=306.1 (MW calc. 305.33); Rₜ=3.13 min.

Step 8:

To a mixture of the intermediate from step 7 (50 mg, 0.16 mmol) and 2,6-difluoro-phenylamine (27 μL, 0.24 mmol) in toluene (3 mL) was added trimethyl aluminium (0.4 mL, 0.8 mmol, 2M in toluene) at 0° C. drop-wise and the resulting RM was heated at reflux for 30 min. The RM was quenched with sat. NH₄Cl solution and extracted with EtOAc. The organic layer was dried and concentrated under reduced pressure to give the crude product which was purified by CC (SiO₂; 20% EtOAc/Hex) to yield the title compound.

LC-MS (Method 2): m/z [M+H]⁺=389.1 (MW calc. 388.37); Rₜ=0.72 min.

¹H NMR (DMSO-d₆, 400 MHz), δ (ppm)=9.69 (s, 1H), 7.97 (s, 1H), 7.4-7.35 (m, 1H), 7.16 (t, 2H, J=8.02 Hz), 6.60 (s, 1H), 3.91 (s, 3H), 3.85 (s, 3H), 3.67 (s, 3H), 2.04 (s, 3H).

### Example 65

### N-(3,5-Difluoro-pyridin-4-yl)-5-(4,6-dimethoxy-pyridin-3-yl)-1,4-dimethyl-1H-pyrazole-3-carboxylic acid amide

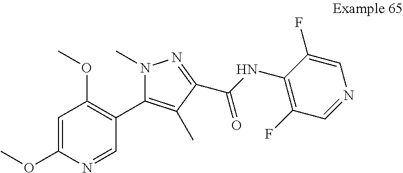 [SMILES: COc1cc(OC)c(-c2c(C)c(C(=O)Nc3c(F)cncc3F)nn2C)cn1]

The title compound was prepared in analogy to example 64 (80 mg, 62%).

LC-MS (Method 2): m/z [M+H]⁺=390.1 (MW calc. 381.43); Rₜ 0.65 min.

¹H NMR (DMSO-d₆, 400 MHz), δ (ppm)=10.19 (s, 1H), 8.57 (s, 2H), 7.98 (s, 1H), 6.60 (s, 1H), 3.92 (s, 3H), 3.85 (s, 3H), 3.69 (s, 3H), 2.05 (s, 3H).

### Example 66

### 5-(4,6-Dimethoxy-pyridin-3-yl)-N-(4,6-dimethyl-pyridin-3-yl)-1,4-dimethyl-1H-pyrazole-3-carboxylic acid amide

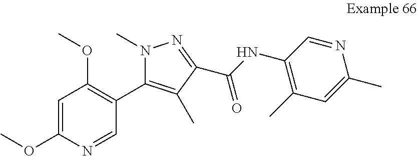

The title compound was prepared in analogy to example 64 (120 mg, 48%).

LC-MS (Method 2): m/z [M+H]⁺=382.2 (MW calc. 381.43); Rₜ=0.47 min.

¹H NMR (DMSO-d₆, 400 MHz), δ (ppm)=9.61 (s, 1H), 8.38 (s, 1H), 7.97 (s, 1H), 7.14 (s, 1H), 6.60 (s, 1H), 3.91 (s, 3H), 3.85 (s, 3H), 3.67 (s, 3H), 2.41 (s, 3H), 2.20 (s, 3H), 2.05 (s, 3H).

### Example 67

### 5-(4,6-Dimethoxy-pyridin-3-yl)-N-(5-fluoro-4-methyl-pyridin-3-yl)-1,4-dimethyl-1H-pyrazole-3-carboxylic acid amide

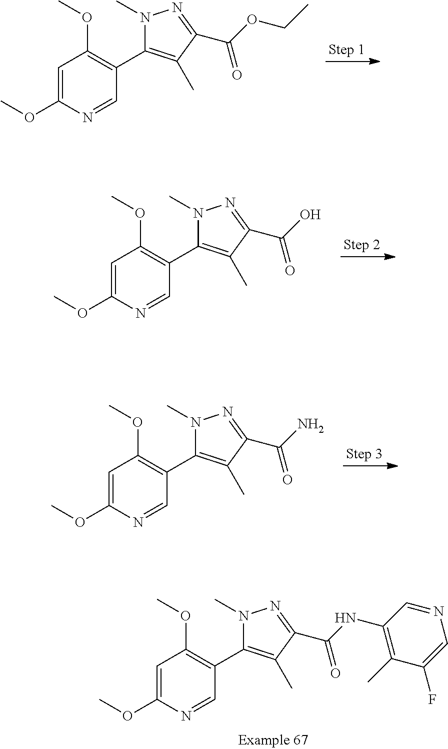

Step 1:

To a solution of 5-(4,6-dimethoxy-pyridin-3-yl)-1,4-dimethyl-1H-pyrazole-3-carboxylic acid ethyl ester (350 mg, 1.15 mmol) in a mixture of MeOH:THF:H₂O (1:1:1, 12 mL) was added LiOH.H₂O (145 mg, 3.44 mmol) at 0° C. and the resulting RM was stirred at RT for 4 h. The RM was concentrated under reduced pressure, diluted with water and washed with CH₂Cl₂. The aqueous layer was acidified with sat.NaHSO₄ solution up to pH˜3 and extracted with CH₂Cl₂. The organic layer was dried and concentrated under reduced pressure to give 5-(4,6-dimethoxy-pyridin-3-yl)-1,4-dimethyl-1H-pyrazole-3-carboxylic acid (270 mg, 84%).

LC-MS (Method 4): m/z [M+H]⁺=278.3 (MW calc. 277.28); Rₜ=1.66 min.

Step 2:

To a cooled solution of the intermediate from step 1 (270 mg, 0.97 mmol) in CH₂Cl₂ (10 mL) were added oxalyl chloride (0.25 ml, 2.92 mmol) and DMF (2 drops). The resulting RM was stirred at RT for 2 h. The RM was concentrated under reduced pressure in inert atmosphere and the residue was diluted with with CH₂Cl₂ (10 mL). NH₃ gas was passed through the RM for 5 min at 0° C. and the resulting RM was stirred at RT for 1 h. The RM was concentrated and diluted with water and extracted with CH₂Cl₂. The combined organic layers were dried and concentrated under reduced pressure to give the desired compound (240 mg, 89%) which was used in the next step without purification.

LC-MS (Method 4): m/z [M+H]⁺=277.1 (MW calc. 276.29); Rₜ=2.50 min.

Step 3:

A mixture of the intermediate from step 2 (150 mg, 0.54 mmol) and K₂CO₃ (150 mg, 1.09 mmol,) in toluene (5 mL) was degassed with Ar for 30 min. 3-bromo-5-fluoro-4-methyl-pyridine (97 μL, 0.815 mmol), CuI (5 mg, 0.027 mmol) and N,N′-dimethylethylenediamine (10 μL, 0.092 mmol) were added to the RM and heated at 100° C. in a sealed tube for 16 h. The RM was concentrated under reduced pressure, diluted with water and extracted with EtOAc. The organic layer was dried and concentrated under reduced pressure to give the crude product which was purified by CC (SiO₂; 1% MeOH in CH₂Cl₂) to yield the title compound (115 mg, 55%).

LC-MS (Method 2): m/z [M+H]⁺=386.2 (MW calc. 385.39); Rₜ=0.66 min.

¹H-NMR (DMSO-d₆, 400 MHz), δ (ppm)=9.94 (s, 1H), 8.48 (s, 1H), 8.36 (s, 1H), 7.98 (s, 1H), 6.60 (s, 1H), 3.91 (s, 3H), 3.85 (s, 3H), 3.69 (s, 3H), 2.19 (d, 3H, J=0.88 Hz), 2.06 (s, 3H).

### Example 68

### N-(2,6-Difluoro-phenyl)-5-(6-ethoxy-4-methoxy-pyridin-3-yl)-1H-pyrazole-3-carboxylic acid amide hydrochloride

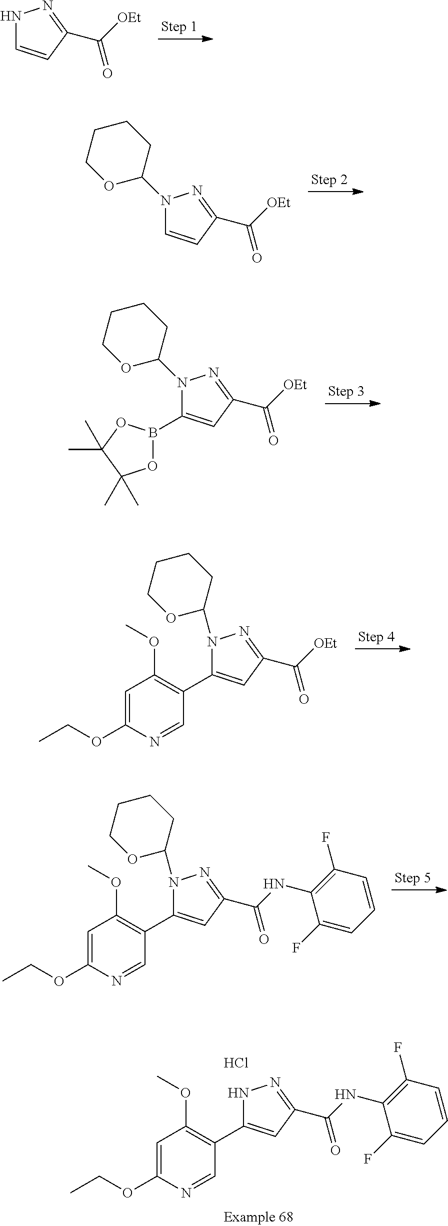

Step 1:

To a solution of ethyl 1H-pyrazole-3-carboxylate (1.80 g, 12.8 mmol) in toluene (12 mL) were added 3,4-dihydro-2H-pyran (1.13 mL, 13.5 mmol) and TFA (14 μL, 128 μmol) and the mixture was stirred at 80° C. for 2 h. The volatiles were removed under reduced pressure and the residue was dissolved in EtOAc and was washed with aqueous Na₂CO₃ and brine. The organic layer was dried and the volatiles were removed under reduced pressure to give the desired compound which was used in the next step without further purification (2.6 g, 90%).

Step 2: 4,4′-Di-tert-butyl-2,2′-dipyridyl (40 mg) was added to a solution of (1,5-Cycloocta-diene)(methoxy)iridium(1) dimer (57 mg) and pinacolborane (1.14 g) in pentane (3 mL) and the mixture was stirred for 20 min at RT. Then a solution of step 1 intermediate (1.00 g, 4.49 mmol) in pentane (0.7 mL) and THF (1.4 mL) was added and the solution was stirred at RT for 3 d. The volatiles were removed under reduced pressure and the residue was purified by CC (SiO₂, CH₂Cl₂/CH₃OH) to yield the desired product (1.4 g, 90%).

¹H-NMR (600 DMSO-d₆): δ=7.04 (s, 1H), 5.76 (dd, J=10.0, 2.5 Hz, 1H), 4.28 (qd, J=7.1, 2.8 Hz, 2H), 3.97-3.90 (m, 1H), 3.60 (ddd, J=14.0, 7.3, 5.0 Hz, 1H), 2.25 (tdd, J=12.7, 9.9, 4.1 Hz, 1H), 2.00 (dtd, J=13.1, 4.0, 1.7 Hz, 1H), 1.98-1.91 (m, 1H), 1.70-1.60 (m, 1H), 1.55 (tq, J=8.4, 4.0 Hz, 2H), 1.34-1.26 (m, 15H) ppm.

Step 3:

A mixture of BB-32 (500 mg, 2.15 mmol), step 2 intermediate (1.21 g, 4.31 mmol), LiOH (48 mg, 2.40 mmol) and bis(tri-tert-butylphosphine)palladium(0) (110 mg, 215 μmol) in dry DMF was stirred under N₂ at 80° C. for 1 h. The volatiles were removed under reduced pressure and the residue was purified by chromatography (Interchim® cartridge50SiHP/25 g, Cy/EtOAc) to yield the desired compound.

Step 4:

A solution of 2,6-difluroaniline (413 mg, 3.20 mmol) in dry toluene (3 mL) was treated with AlMe₃ (2M in tolene, 1.28 mL, 3.20 mmol) and the mixture was stirred at 0° C. for 30 min. A solution of step 3 intermediate (399 mg, 1.07 mmol) in toluene (7 mL) was added and the mixture was stirred at 120° C. for 1 h. Aqueous NaOH (1M) was carefully added and the mixture was extracted with EtOAc. The combined organic layers were dried and the volatiles were removed under reduced pressure to give a crude which was used in the next step without further purification (260 mg, 53%).

Step 5:

The intermediate of step 4 (260 mg, 567 mmol) was treated with HCl (1.25M in MeOH, 6.8 mL, 8.5 1 mmol) and the mixture was stirred for 2 at rt. tert-butyl methyl ether was added and the precipitating solid was isolated by filtration. The solid was washed with tert-butyl methyl ether and was dried under reduced pressure to give the desired compound (102 mg, 44%).

LC-MS (Method 2): m/z [M+H]+=375.1 (MW [M−HCl]⁺ calc.=374.34); Rt=0.64 min.

¹H-NMR (600 MHz, DMSO-d6, 400 MHz), δ (ppm)=9.86 (s, 1H), 8.42 (s, 1H), 7.45-7.37 (m, 1H), 7.21 (t, J=8.0 Hz, 2H), 7.14 (s, 1H), 6.55 (s, 1H), 4.40-4.32 (m, 2H), 3.95 (s, 3H), 1.34 (t, J=7.1 Hz, 3H).

### Example 69

### N-(2,6-Difluoro-phenyl)-5-(4,6-dimethoxy-pyridin-3-yl)-1H-pyrazole-3-carboxylic acid amide hydrochloride

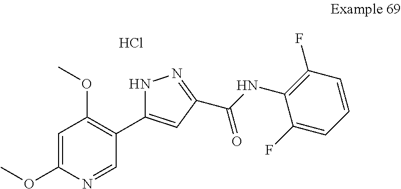 [SMILES: COc1cc(OC)c(-c2cc(C(=O)Nc3c(F)cccc3F)n[nH]2)cn1 ; Cl]

The title compound was prepared in analogy to example 68.

LC-MS (Method 2): m/z [M+H]+=361.1 (MW [M−HCl]⁺ calc.=360.31); Rt=0.60 min.

¹H-NMR (DMSO-d₆, 400 MHz), δ (ppm)=9.87 (s, 1H), 8.44 (s, 1H), 7.48-7.35 (m, 1H), 7.20 (t, J=8.1 Hz, 2H), 7.15 (s, 1H), 6.57 (s, 1H), 3.96 (s, 3H), 3.91 (s, 3H) ppm.

### Example 70

### N-(2,6-Difluoro-phenyl)-5-(6-hydroxy-4-methoxy-pyridin-3-yl)-1-methyl-1H-pyrazole-3-carboxylic acid amide

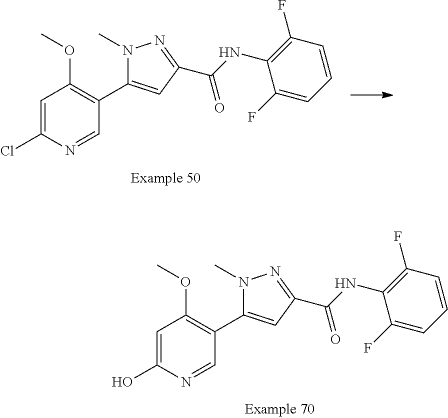 [SMILES: COc1cc(Cl)ncc1-c1cc(C(=O)Nc2c(F)cccc2F)nn1C ; COc1cc(O)ncc1-c1cc(C(=O)Nc2c(F)cccc2F)nn1C]

To a mixture of the step 1 intermediate of example 50 (30 mg, 79 μmol), KOH (12 mg, 238 μmol), 2-di-tert-butylphosphino-2′,4′,6′-triisopropylbiphenyl (1 mg) and water (28 μL) under N₂ was added a solution of chloro[2-(di-tert-butylphosphino)-2′,4′,6′-triisopropyl-1,1′-biphenyl][2-(2-aminoethyl)phenyl)]palladium(II) (1 mg) in dioxane (168 μL) and the mixture was stirred at 80° C. overnight. Aqueous HCl (1M) was added, the mixture was neutralized through addition of aqueous NaHCO₃ and was extracted with EtOAc. The combined organic layers were dried and the volatiles were removed under reduced pressure. The residue was purified through CC (SiO₂) to yield the desired compound (10 mg, 35%).

LC-MS (Method 2): m/z [M+H]+=361.1 (MW [M−HCl]⁺ calc.=360.31); Rt=0.47 min.

¹H-NMR (DMSO-d₆, 400 MHz), δ (ppm)=9.84 (m, 1H), 7.44 (s, 1H), 7.39 (tt, J=8.5, 6.3 Hz, 1H), 7.21-7.13 (m, 2H), 6.73 (s, 1H), 5.89 (s, 1H), 3.78 (s, 3H), 3.74 (s, 3H) ppm.

### Example 71

### N-(2,6-Difluoro-phenyl)-5-(6-ethoxy-4-hydroxy-pyridin-3-yl)-1-methyl-1H-pyrazole-3-carboxylic acid amide

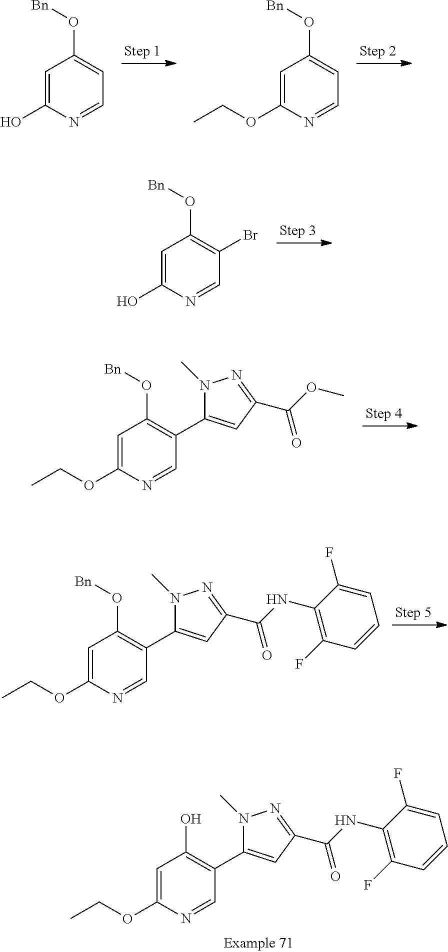

Step 1:

A mixture of 4-benzyloxy-pyridin-2-ol (5.0 g, 24.87 mmol), ethyl iodide (10.05 mL, 124.35 mmol) and Ag₂CO₃ (8.91 g, 32.33 mmol) in CHCl₃ (170 mL) was stirred at RT for 16 h. The RM was filtered and filtrate was concentrated under reduced pressure to give the crude product which was purified by CC (SiO₂; 10% EtOAc/Hex) to yield 4-benzyloxy-2-ethoxy-pyridine (2.2 g, 38%).

LC-MS (Method 4): m/z [M+H]⁺=229.9 (MW calc. 229.27); Rₜ=3.57 min.

Step 2:

A mixture of 4-benzyloxy-2-ethoxy-pyridine (500 mg, 2.18 mmol) and NBS (389 mg, 2.18 mmol) in CH₃CN (110 mL) was stirred in the dark at 80° C. for 16 h. The RM was concentrated and diluted with EtOAc and washed with water. The organic layer was dried and concentrated under reduced pressure to give the crude product which was purified by CC (SiO₂; 2% EtOAc/Hex) to yield 4-benzyloxy-5-bromo-2-ethoxy-pyridine (350 mg, 52%).

LC-MS (Method 4): m/z [M+H]⁺=308.1 & 310.1 (MW calc. 308.17); Rₜ=3.92 min.

Step 3:

A mixture of 4-benzyloxy-5-bromo-2-ethoxy-pyridine (500 mg, 1.63 mmol) and BB-4a (563 mg, 2.12 mmol) in dioxane (20 mL) was degassed with Ar for 10 min. KF (284 mg, 4.89 mmol) and bis(tri-tert butyl phosphine) Pd(0) (417 mg, 0.815 mmol) were added to the RM and heated at 100° C. for 4 h. The RM was concentrated to give the crude product which was purified by CC (SiO₂; 25% EtOAc/Hex) to yield the desired product (300 mg, 50%).

LC-MS (Method 4): m/z [M+H]⁺=368.0 (MW calc. 367.40); Rₜ=3.43 min.

Step 4:

The desired compound was prepared in analogy to previous examples (185 mg, 45%).

LC-MS (Method 4): m/z [M+H]⁺=465.0 (MW calc. 464.46); Rₜ=4.60 min.

Step 5:

A solution of the intermediate from step 4 (175 mg, 0.377 mmol) in MeOH (14 mL) was degassed with Ar for 30 min. Subsequently Pd—C(87 mg, 50% w/w) was added to the RM and the resulting RM was stirred under H₂ balloon pressure at RT for 2 h. The catalyst was filtered off and washed with methanol (25 mL). The filtrate was concentrated under reduced pressure to give the crude product which was triturated with 20% CH₂Cl₂ in Hex to yield the title compound (90 mg, 63%).

LC-MS (Method 2): m/z [M+H]⁺=375.1 (MW calc. 374.34); Rₜ=0.49 min.

¹H-NMR (DMSO-d₆, 400 MHz), δ (ppm)=11.25 (bs, 1H), 9.83 (s, 1H), 7.97 (s, 1H), 7.42-7.36 (m, 1H), 7.17 (t, 2H, J=8.0 Hz), 6.75 (s, 1H), 6.29 (s, 1H), 4.30 (q, 2H, J=6.8 Hz), 3.77 (s, 3H), 1.31 (t, 3H, J=7.0 Hz).

### Example 72

### 5-[3-Cyano-6-(trifluoromethyl)-pyridin-2-yl]-N-(2,6-difluoro-phenyl)-1-methyl-1H-pyrazole-3-carboxylic acid amide

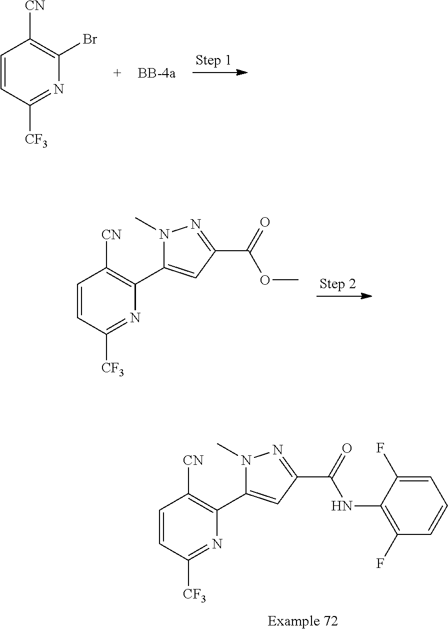 [SMILES: N#Cc1ccc(C(F)(F)F)nc1Br ; COC(=O)c1cc(-c2nc(C(F)(F)F)ccc2C#N)n(C)n1 ; Cn1nc(C(=O)Nc2c(F)cccc2F)cc1-c1nc(C(F)(F)F)ccc1C#N]

The title compound was synthesized in analogy to example 42 in 2 steps starting from commercially available 2-bromo-6-frifluoromethylnicotinnitrile (200 mg, 797 μmol) and BB-4a (446 mg, 1.59 mmol) (32 mg, 17% over 2 steps).

LC-MS (Method 2): m/z [M+H]⁺=408.1 (MW calc.=407.3); Rt=0.74 min.

¹H-NMR (600 MHz, CDCl₃) δ=10.10 (s, 1H), 8.87 (d, J=8.1 Hz, 1H), 8.24 (d, J=8.2 Hz, 1H), 7.53 (s, 1H), 7.42 (tt, J=8.5, 6.3 Hz, 1H), 7.21 (t, J=8.1 Hz, 2H), 4.16 (s, 3H) ppm.

### Example 73

### 5-(6-tert-Butyl-3-cyano-pyridin-2-yl)-N-(2,6-difluoro-phenyl)-1-methyl-1H-pyrazole-3-carboxylic acid amide

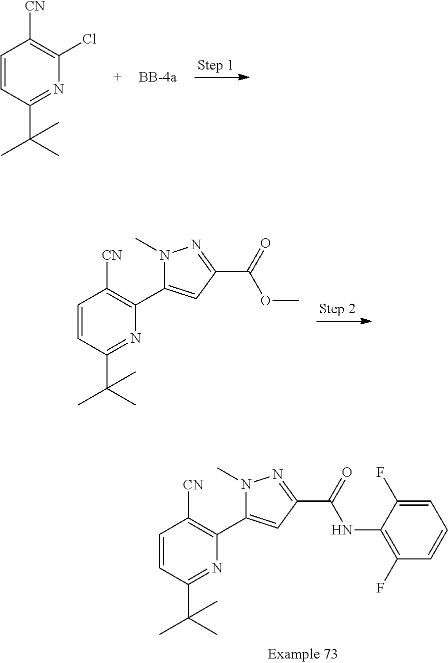 [SMILES: CC(C)(C)c1ccc(C#N)c(Cl)n1 ; COC(=O)c1cc(-c2nc(C(C)(C)C)ccc2C#N)n(C)n1 ; Cn1nc(C(=O)Nc2c(F)cccc2F)cc1-c1nc(C(C)(C)C)ccc1C#N]

The title compound was synthesized in analogy to example 42 in 2 steps starting from commercially available 6-tert-butyl-2-chloronicotinonitrile (100 mg, 514 μmol) and BB-4a (215 mg, 770 μmol) (35 mg, 17% over 2 steps).

LC-MS (Method 2): m/z [M+H]⁺=396.2 (MW calc.=395.4); Rt=0.83 min.

¹H-NMR (600 MHz, CDCl₃) δ=10.04 (s, 1H), 8.44 (d, J=8.4 Hz, 1H), 7.75 (d, J=8.3 Hz, 1H), 7.44-7.36 (m, 2H), 7.20 (t, J=8.1 Hz, 2H), 4.18 (s, 3H), 1.40 (s, 9H).

### Example 74

### N-(2,6-Difluoro-phenyl)-5-[2-methoxy-5-(4-methyl-5-oxo-4H-[1,3,4]oxadiazol-2-yl)-pyridin-3-yl]-1-methyl-1H-pyrazole-3-carboxylic acid amide

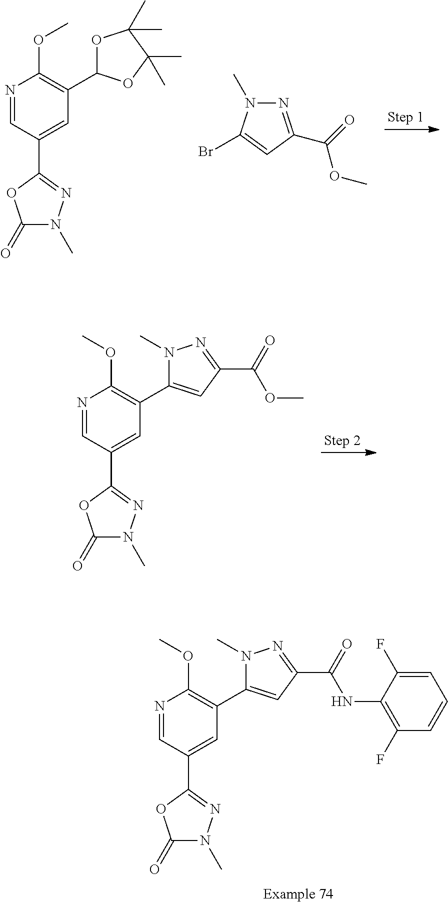 [SMILES: COc1ncc(-c2nn(C)c(=O)o2)cc1C1OC(C)(C)C(C)(C)O1 ; COC(=O)c1cc(Br)n(C)n1 ; COC(=O)c1cc(-c2cc(-c3nn(C)c(=O)o3)cnc2OC)n(C)n1 ; COc1ncc(-c2nn(C)c(=O)o2)cc1-c1cc(C(=O)Nc2c(F)cccc2F)nn1C]

Step 1:

A mixture of 5-(6-methoxy-5-(4,4,5,5-tetramethyl-1,3-dioxolan-2-yl)pyridin-3-yl)-3-methyl-1,3,4-oxadiazol-2(3H)-one (prepared in analogy to WO 2013164769, 240 mg, 720 μmol), methyl 5-bromo-1-methyl-1H-pyrazole-3-carboxylate (473 mg, 2.16 mmol), LiOH (25 mg, 1.08 mmol) and bis(tri-tert-butylphosphine)palladium(0) (73 mg, 144 μmol) in a mixture of DMF (0.4 mL) and THF (3.3 mL) was stirred under N₂ at 85° C. for 2 h. The volatiles were removed under reduced pressure and the residue was purified by chromatography (Interchim® cartridge50SiHP/25 g, Cy/EtOAc) to yield the desired compound (84 mg, 34%).

Step 2:

A solution of 2,6-difluroaniline (299 mg, 2.32 mmol) in dry toluene (0.3 mL) was treated with AlMe₃ (2M in tolene, 0.28 mL, 695 μmol) and the mixture was stirred at 0° C. for 30 min. A solution of step 2 intermediate (80 mg, 232 μmol) in toluene (0.3 mL) was added and the mixture was stirred at 120° C. for 1 h. Aqueous NaOH (1M) was carefully added and the mixture was extracted with EtOAc. The combined organic layers were dried and the volatiles were removed under reduced pressure. The residue was purified by chromatography (Interchim® cartridge50SiHP/12 g, Cy/EtOAc) to yield the desired compound (25 mg, 24%).

LC-MS (Method 2): m/z [M+H]⁺=443.1 (MW calc.=442.4); Rt=0.67 min.

¹H-NMR (600 MHz, CDCl₃) δ=9.91 (s, 1H), 8.74 (dd, J=2.4, 0.7 Hz, 1H), 8.17-8.12 (m, 1H), 7.40 (tt, J=8.5, 6.2 Hz, 1H), 7.23-7.15 (m, 2H), 6.95 (s, 1H), 4.03 (s, 3H), 3.83 (s, 3H), 3.43 (s, 3H).

### Example 75

### N-(2,6-Difluoro-phenyl)-5-(4-dimethylamino-6-methoxy-pyridin-3-yl)-1-methyl-1H-pyrazole-3-carboxylic acid amide

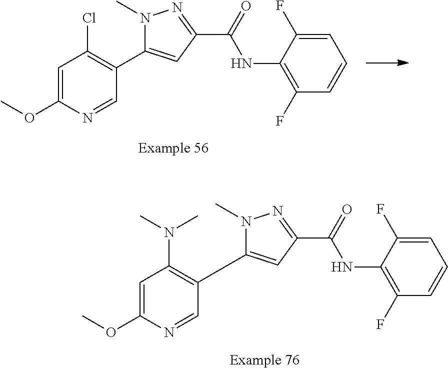 [SMILES: COc1cc(Cl)c(-c2cc(C(=O)Nc3c(F)cccc3F)nn2C)cn1 ; COc1cc(N(C)C)c(-c2cc(C(=O)Nc3c(F)cccc3F)nn2C)cn1]

A mixture of example 56 (153 mg, 406 μmol), dimethyl amine hydrochloride (231 mg, 2.84 mmol) and DIPEA (440 μL, 3.41 mmol) in 2-propanol (3.2 mL) was heated in a microwave to 120° C. for 24 h. The volatiels were removed under reduced pressure and the residue was purified by chromatography (Interchim® cartridge50SiHP/12 g, Cy/EtOAc) to yield the desired compound (42 mg, 27%).

LC-MS (Method 2): m/z [M+H]⁺=388.2 (MW calc.=387.39); Rt=0.46 min.

¹H-NMR (600 MHz, CDCl₃) δ=9.88 (s, 1H), 7.82 (d, J=1.2 Hz, 1H), 7.39 (tt, J=8.4, 6.2 Hz, 1H), 7.18 (t, J=8.0 Hz, 2H), 6.81 (d, J=1.0 Hz, 1H), 6.21 (s, 1H), 3.86 (s, 3H), 3.71 (s, 3H), 2.65 (s, 6H).

The following compounds were synthesized by analogous procedures:

| Ex. | | |
|---|---|---|
| No | Structure | Name |
| 76 | 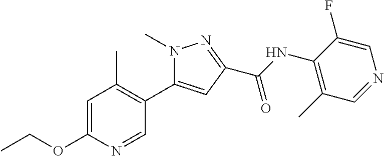 [SMILES: CCOc1cc(C)c(-c2cc(C(=O)Nc3c(C)cncc3F)nn2C)cn1] | 5-(6-Ethoxy-4-methyl-pyridin-3-yl)-N-(3-fluoro- 5-methyl-pyridin-4-yl)-1-methyl-1H-pyrazole- 3-carboxylic acid amide |
| 77 | 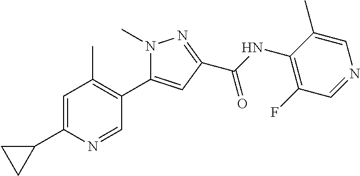 [SMILES: Cc1cc(C2CC2)ncc1-c1cc(C(=O)Nc2c(C)cncc2F)nn1C] | 5-(6-Cyclopropyl-4-methyl-pyridin-3-yl)-N-(3- fluoro-5-methyl-pyridin-4-yl)-1-methyl-1H- pyrazole-3-carboxylic acid amide |
| 78 | 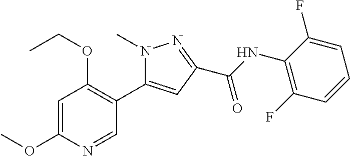 [SMILES: CCOc1cc(OC)ncc1-c1cc(C(=O)Nc2c(F)cccc2F)nn1C] | N-(2,6-Difluoro-phenyl)-5-(4-ethoxy-6- methoxy-pyridin-3-yl)-1-methyl-1H-pyrazole- 3-carboxylic acid amide |
| 79 | 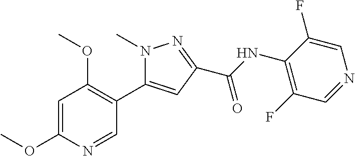 [SMILES: COc1cc(OC)c(-c2cc(C(=O)Nc3c(F)cncc3F)nn2C)cn1] | N-(3,5-Difluoro-pyridin-4-yl)-5-(4,6-dimethoxy- pyridin-3-yl)-1-methyl-1H-pyrazole-3- carboxylic acid amide |
| 80 | 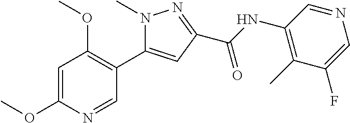 [SMILES: COc1cc(OC)c(-c2cc(C(=O)Nc3cncc(F)c3C)nn2C)cn1] | 5-(4,6-Dimethoxy-pyridin-3-yl)-N-(5-fluoro-4- methyl-pyridin-3-yl)-1-methyl-1H-pyrazole-3- carboxylic acid amide |
| 81 | 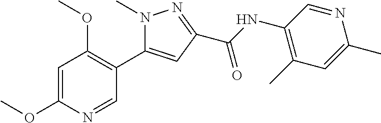 | 5-(4,6-Dimethoxy-pyridin-3-yl)-N-(4,6- dimethyl-pyridin-3-yl)-1-methyl-1H-pyrazole- 3-carboxylic acid amide |
| 82 | 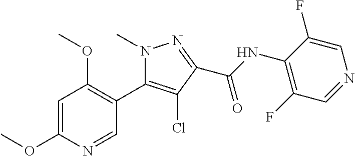 [SMILES: COc1cc(OC)c(-c2c(Cl)c(C(=O)Nc3c(F)cncc3F)nn2C)cn1] | 4-Chloro-N-(3,5-difluoro-pyridin-4-yl)-5-(4,6- dimethoxy-pyridin-3-yl)-1-methyl-1H-pyrazole- 3-carboxylic acid amide |
| 83 | 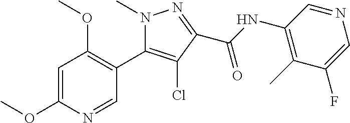 [SMILES: COc1cc(OC)c(-c2c(Cl)c(C(=O)Nc3cncc(F)c3C)nn2C)cn1] | 4-Chloro-5-(4,6-dimethoxy-pyridin-3-yl)-N-(5- fluoro-4-methyl-pyridin-3-yl)-1-methyl-1H- pyrazole-3-carboxylic acid amide |
| 84 | 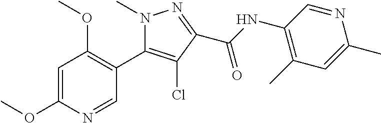 | 4-Chloro-5-(4,6-dimethoxy-pyridin-3-yl)-N- (4,6-dimethyl-pyridin-3-yl)-1-methyl-1H- pyrazole-3-carboxylic acid amide |
| 85 | 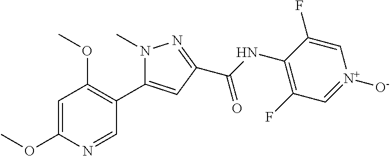 [SMILES: COc1cc(OC)c(-c2cc(C(=O)Nc3c(F)c[n+]([O-])cc3F)nn2C)cn1] | N-(3,5-Difluoro-1-oxo-pyridin-4-yl)-5-(4,6- dimethoxy-pyridin-3-yl)-1-methyl-1H-pyrazole- 3-carboxylic acid amide |
| 86 | 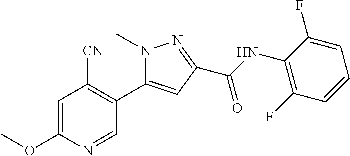 [SMILES: COc1cc(C#N)c(-c2cc(C(=O)Nc3c(F)cccc3F)nn2C)cn1] | 5-(4-Cyano-6-methoxy-pyridin-3-yl)-N-(2,6- difluoro-phenyl)-1-methyl-1H-pyrazole-3- carboxylic acid amide |
| 87 | 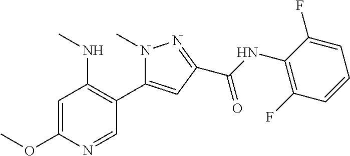 [SMILES: CNc1cc(OC)ncc1-c1cc(C(=O)Nc2c(F)cccc2F)nn1C] | N-(2,6-Difluoro-phenyl)-5-(6-methoxy-4- methylamino-pyridin-3-yl)-1-methyl-1H- pyrazole-3-carboxylic acid amide |
| 88 | 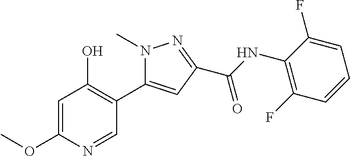 [SMILES: COc1cc(O)c(-c2cc(C(=O)Nc3c(F)cccc3F)nn2C)cn1] | N-(2,6-Difluoro-phenyl)-5-(4-hydroxy-6- methoxy-pyridin-3-yl)-1-methyl-1H-pyrazole- 3-carboxylic acid amide |

### Pharmacological Methods

Compounds of the invention have been tested for their effects on CRAC channels according to the following or similar procedures.

### HEK Calcium Influx Assay

The effect of compounds of the invention on intracellular [Ca²⁺] was tested in the HEK293 cell line (ECACC). HEK293 cells were cultured in DMEM/F12/Glutamax (Gibco) containing 10% FCS (Gibco), and maintained at 37° C., 5% CO₂. Cell were split twice a week [3*10⁶ (Mon-Thu) and 1*10⁶ (Thu-Mon) cells/50 ml medium in T-175 ZK culture flasks, respectively]. Twenty four hours pre-experiment, cells were seeded on 96 well plates (Poly-D-Lysine 96well Black/Clear Plate, BD Biocoat REF 356640) at a density of 40,000 cells/well in DMEM/F12 (Gibco) containing 10% FCS (Gibco), and maintained at 37° C., 5% CO₂. Prior to store-depletion, cell culture medium was removed and cells were loaded with the a Calcium-sensitive fluorescent dye comprised within the Calcium-4-assay kit (Molecular Devices) in nominally Ca²⁺-free HBS buffer (140 mM NaCl, 4 mM KCl, 0.2 mM MgCl₂, 11 mM D-glucose, and 10 mM HEPES, pH 7.4) according to manufacturer's instruction for 60 min at 37° C., 5% CO₂. Passive depletion of intracellular Ca²⁺-stores was then triggered by employing the SERCA inhibitor thapsigargin (2 μM final) for 10 min in the dark (RT). To prevent immediate Ca²⁺-entry via the activated Store-operated channels (SOCs), cells were maintained in Ca²⁺-free HBS buffer comprising 100 μM EGTA during store-depletion. Intracellular changes in [Ca²⁺] were subsequently monitored with the FLIPR device (Molecular Devices). In brief, baseline imaging post-store depletion was allowed for 1 min before adjusting the extracellular buffer to 3 mM CaCl₂. Increases in intracellular [Ca²⁺] due to pre-activated SOC channels were monitored for 15 min until intracellular Ca²⁺ levels had declined into a steady-state. Finally, compounds were administered and Ca²⁺ signals were recorded for additional 10 min. Inhibition of endogenous SOC in HEK293 cells was quantified employing the average Ca²⁺ signal measured from 9.5-10 min post-administration. Zero percent inhibition (MAX) was defined as the Ca²⁺ signal recorded from wells to which DMSO-only had been added instead of compound. Hundred percent inhibition (MIN) was defined as the signal obtained from wells in which cells haven't been treated with TG prior to Ca²⁺ addition and to which DMSO-only had been added instead of compound. For routine IC50 determinations of compounds, 8 concentrations of a serial dilution (1:3.16) were tested, starting off from 10 μM. Reliable IC50's could consequently be determined only, if they were at least sub 2.5-3 μM.

### Jurkat IL-2 Production Assay

The effect of compounds of the invention on Interleukin-2 (IL-2) production/release was tested in the Jurkat T cell line (ECACC) clone E6-1. Jurkat T cells were cultured in DMEM/F12/Glutamax (Gibco) containing 10% FCS (Gibco), and maintained at 37° C., 5% CO₂. Cell were split twice a week [5*10⁶ (Mon-Thu) and 1*10⁷ (Thu-Mon) cells/50 ml medium in T-175 ZK culture flasks, respectively]. Prior to experiment, cells were seeded on 96 well plates (Cellstar 96 Well; Cat No. 655180, Greiner bio-one) at a density of 5*10⁵ cells/well in DMEM/F12/Glutamax (Gibco) containing 10% FCS (Gibco), and incubated for 60 min at 37° C., 5% CO₂. Subsequently, compounds were added and cells were allowed to incubate for 30 min at 37° C., 5% CO₂. Cells were then stimulated with 15 μg/ml Phytohemagglutinin (PHA; Sigma) for 22 hours at 37° C., 5% CO₂. Before sampling of the supernatants, cells were spun down (200*g/5 min/RT). The amount of IL-2 released into the supernatant was quantified with the human IL-2 AlphaLisa kit (Perkin Elmer) according to manufacturer's instructions. Luminescence proximity measurements were carried out in the Synergy H4 reader (BioTek) employing the fluorescence setting of the reader (ex: 680/30 nm; em: 620/40 nm). Inhibition of IL-production/release in/from Jurkat T cells cells was quantified as follows: Zero percent inhibition (MAX) was defined as the [IL-2] determined in supernatants derived from cells to which PHA & DMSO-only had been added instead of compound. Hundred percent inhibition (MIN) was defined as the [IL-2] determined in supernatants derived from cells that had been pre-treated with 1 μM CyclosporineA (Sigma) before the addition of 15 μg/ml PHA. For routine IC50 determinations of compounds, 8 concentrations of a serial dilution (1:3.16) were tested, starting off from 10 μM.

Exemplary compounds of the invention exhibit inhibition of the CRAC channel and inhibition of the IL-2 production in these assays within the following ranges: IC₅₀ values from <0.5 μM (A); 0.5 1.0 μM (B); 1.0 5.0 μM (C) and full IC₅₀ not determined (n.d.). or % inhibition @ 10 μM<50 (C), 50 70 (B), >70 (A) (table 1)

| | % inhib. | |
|---|---|---|
| Example | [@ 10 μM] | IC₅₀ [μM] |
| No. | FLIPR | IL-2 |
| 2 | A | B |
| 3 | A | A |
| 5 | A | C |
| 6 | A | C |
| 7 | B | C |
| 8 | A | C |
| 9 | A | B |
| 10 | A | B |
| 11 | A | A |
| 12 | A | A |
| 13 | A | B |
| 14 | A | A |
| 15 | A | A |
| 16 | A | A |
| 17 | A | A |
| 18 | — | C |
| 19 | A | n.d. |
| 20 | A | A |
| 23 | A | C |
| 28 | A | C |
| 31 | A | C |
| 32 | A | B |
| 33 | A | C |
| 34 | A | C |
| 35 | C | C |
| 36 | B | C |
| 37 | A | C |
| 38 | A | A |
| 41 | C | n.d. |
| 42 | — | B |
| 43 | A | A |
| 44 | A | A |
| 45 | A | A |
| 46 | A | A |
| 47 | A | A |
| 48 | A | B |
| 49 | B | C |
| 51 | B | C |
| 52 | — | B |
| 53 | — | B |
| 54 | A | A |
| 55 | A | A |
| 56 | A | B |
| 57 | A | C |
| 58 | — | C |
| 59 | B | n.d. |
| 60 | A | B |
| 61 | A | A |
| 62 | B | C |
| 63 | A | C |
| 64 | A | C |
| 65 | A | C |
| 66 | B | C |
| 67 | A | C |
| 68 | A | C |
| 69 | A | C |
| 70 | C | n.d. |
| 71 | C | n.d. |
| 72 | C | n.d. |
| 73 | C | n.d. |
| 74 | C | C |
| 75 | — | C |

It was surprisingly found that the specific substitution pattern at the pyridinyl-moiety according to the present invention has a significant influence on the pharmacological activity as demonstrated by structural analogs according to general formula (Ib) in table 2 when compared to pyridine compounds which do not possess a substituent in ortho-position to nitrogen of the pyridine moiety as known from WO 2014/108336 (comparative examples #1 and #2):

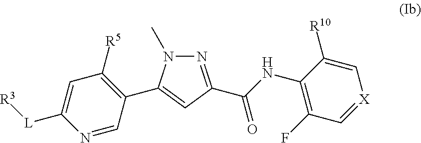

TABLE 2
(each with R⁵ = CH₃; R¹⁰ = F)

| Example | | | inhib. [@ 10 |
|---|---|---|---|
| No. | R³L | X | μM] FLIPR |
| Comparative | H | CH | 32% |
| example #1 | | | |
| 3 | C₂H₅O | CH | 75% |
| 6 | C₂H₅ | CH | 67% |
| 8 | 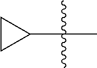 [SMILES: *C1CC1] | CH | 81% |
| 10 | 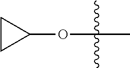 [SMILES: *OC1CC1] | CH | 83% |
| 12 | CF₃O | CH | 92% |
| 13 | 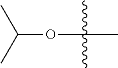 [SMILES: *OC(C)C] | CH | 100% |
| 19 | 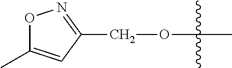 [SMILES: *OCc1cc(C)on1] | CH | 113% |
| 23 | 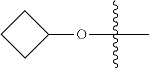 [SMILES: *OC1CCC1] | CH | 94% |
| 28 | CH₃OC₂H₅O | CH | 87% |
| 37 | (CH₃)₂N | CH | 98% |
| 59 | CH₃OCH₂ | CH | 67% |
| 60 | CF₃ | CH | 100% |
| Comparative | H | N | 34% |
| example #2 | | | |
| 2 | C₂H₅O | N | 95% |
| 7 | C₂H₅ | N | 61% |
| 9 | 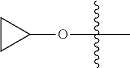 [SMILES: *OC1CC1] | N | 76% |
| 11 | CF₃O | N | 94% |
| 20 | 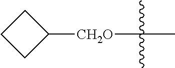 | N | 121% |

Comparative examples #1 and #2 have been prepared in analogy to procedures according to WO 2014/108336.

Furthermore, the compounds according to the invention proved to be beneficial with respect to their physicochemical properties. In table 3, exemplary nephelometric solubility data were provided in comparison with analogous compounds known from the prior art US 2006/0173006 (comparative example #3 and #4) and WO 2013/164769 (comparative example #5).

### Nephelometric Solubility Measurement

BMG Labtech Nephelostar (S/N 504-0167), 37° C.; Gain: 100; Laser Beam Focus 1.5 mm; Laser intensity 80%; measurement time/well 0.1 s; positioning delay 0.1 s; data analysis: BMG Labtech MARS software

Measurements with BMG Labtech Nephelostar performed though addition of DMSO stock solutions of compound (2, 5, 10, 20, 30, 40, 50, 60, 80, 100 [μg/mL]) into phosphate buffered saline (pH 7.4, purchased from Invitrogen, catalogue number 10010-056); incubation time: 2 minutes [blank measurement contains DMSO (2.5 μL) and buffered saline (47.5 μL)]; Detection of insoluble particles in liquid samples was performed by measuring forward scattered light.

TABLE 3

| | | Nephelometric |
|---|---|---|
| Example No. | Compound | solubility [μM] |
| Comparative example #3 | 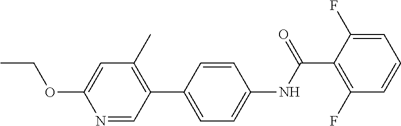 [SMILES: CCOc1cc(C)c(-c2ccc(NC(=O)c3c(F)cccc3F)cc2)cn1] | <14 |
| 3 | 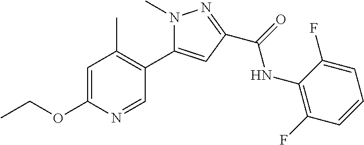 [SMILES: CCOc1cc(C)c(-c2cc(C(=O)Nc3c(F)cccc3F)nn2C)cn1] | 149.2 |
| Comparative example #4 | 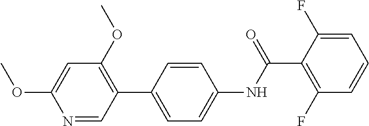 [SMILES: COc1cc(OC)c(-c2ccc(NC(=O)c3c(F)cccc3F)cc2)cn1] | 38.8 |
| 38 | 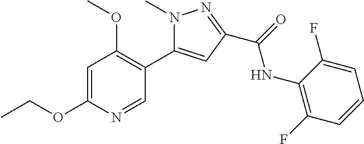 [SMILES: CCOc1cc(OC)c(-c2cc(C(=O)Nc3c(F)cccc3F)nn2C)cn1] | >267 |
| Comparative example #5 (WO 2013/164769, example 6) | 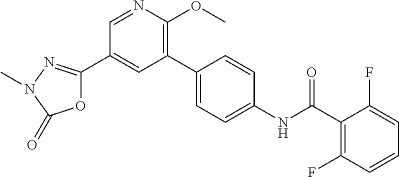 [SMILES: COc1ncc(-c2nn(C)c(=O)o2)cc1-c1ccc(NC(=O)c2c(F)cccc2F)cc1] | <11 |
| 74 | 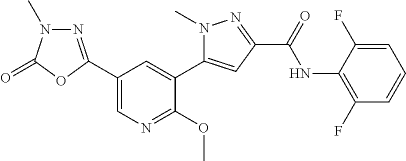 [SMILES: COc1ncc(-c2nn(C)c(=O)o2)cc1-c1cc(C(=O)Nc2c(F)cccc2F)nn1C] | 130.7 |

1. A compound of formula (I): 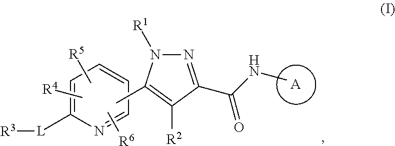
wherein
R¹ denotes H, C₁₋₄-alkyl or C₃₋₆-cycloalkyl;
R² denotes H; F; Cl; Br; CN; CF₃; CF₂H; CFH₂; C₁₋₄-alkyl; OH; O—C₁₋₄-alkyl; NH₂; N(H)C₁₋₄-alkyl; or N(C₁₋₄-alkyl)₂;
L represents bond, O, C₁₋₄-alkylene, C₁₋₄-alkylene-O or O—C₁₋₄-alkylene;
R³ is selected from the group consisting of Cl, OH, CN; CF₃; CF₂H; CFH₂; C₁₋₄-alkyl; C₃₋₆-cycloalkyl; 3 to 7 membered heterocycloalkyl; 5- to 6-membered heteroaryl; C(═O)OH; C(═O)O—C₁₋₄-alkyl; C(═O)NH₂; C(═O)N(H)C₁₋₄-alkyl; C(═O)N(C₁₋₄-alkyl)₂; O—C₁₋₄-alkyl; OCF₃; OCF₂H; OCFH₂; NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl); N(C₁₋₄-alkyl)S(═O)₂(C₁₋₄-alkyl); S(═O)₂C₁₋₄-alkyl; S(═O)C₁₋₄-alkyl; S(═O)₂NH₂; S(═O)₂N(H)C₁₋₄-alkyl; and S(═O)₂N(C₁₋₄-alkyl)₂;
R⁴, R⁵ and R⁶ are independently selected from the group consisting H; F; Cl; Br; CN; CF₃; CF₂H; CFH₂; C₁₋₄-alkyl; C₃₋₆-cycloalkyl; 3 to 7 membered heterocycloalkyl; OH; O—C₁₋₄-alkyl; OCF₃; OCF₂H; OCFH₂; OC₃₋₆-cycloalkyl; O-(3 to 7 membered heterocycloalkyl); NH₂; N(H)C₁₋₄-alkyl; N(C₁₋₄-alkyl)₂; and NH(C═O)(C₁₋₄-alkyl);
and
A represents phenyl or 5- to 6-membered heteroaryl,
wherein
said heteroaryl, cycloalkyl and heterocycloalkyl each independently are unsubstituted or mono- or polysubstituted; and
wherein
said C₁₋₄-alkyl and C₁₋₄-alkylene each independently are linear or branched, and each independently are unsubstituted or mono- or polysubstituted;
optionally in the form of a single stereoisomer or a mixture of stereoisomers, in the form of the free compound and/or a physiologically acceptable salt thereof and/or a physiologically acceptable solvate thereof.

2. The compound according to claim 1, which has the formula (Ia): 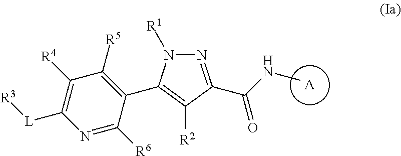
wherein R¹, R², R³, R⁴, R⁵, R⁶, L and A are defined as in claim 1.

3. The compound according to claim 1, wherein R² is selected from the group consisting of H; F; Cl; Br; CN; CF₃; CF₂H; CFH₂; CH₃; CH₂CH₃; CH(CH₃)₂; cyclopropyl; OH; OCH₃; OCF₃; OCF₂H; OCFH₂; NH₂; N(H)CH₃; and N(CH₃)₂.

4. The compound according to claim 1, wherein R¹ is selected from the group consisting of unsubstituted C₁₋₄-alkyl and unsubstituted cyclopropyl.

5. The compound according to claim 1, wherein A is selected from the group consisting of
phenyl, pyridinyl, pyrazinyl, pyrimidinyl, pyridazinyl; triazinyl and the N-oxide of said pyridinyl, pyrazinyl, pyrimidinyl, pyridazinyl or triazinyl,
each unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; and NH(C═O)(C₁₋₄-alkyl).

6. The compound according to claim 1, wherein A has substructure (II): 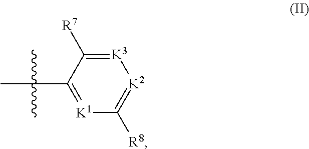
wherein
K¹ stands for N or CR⁷; K² stands for N or CR⁸ or N⁺—O⁻; and K³ stands for N or CR⁸;
each R⁷ independently is selected from H; F; Cl; Br; CN; CF₃; CF₂H; CFH₂; C₁₋₄-alkyl; and C₃₋₆-cycloalkyl;
and each R⁸ is independently selected from the group consisting of H; F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; and NH(C═O)(C₁₋₄-alkyl).

7. The compound according to claim 1, wherein A is selected from the group consisting of
2,6-difluorophenyl, 5-fluoro-4-methyl-pyridin-3-yl, 3-fluoro-pyridin-4-yl, 2,4-dimethyl-pyridin-5-yl, 3,5-difluoro-pyridin-4-yl, 3,5-difluoro-pyridin-2-yl, 3,5-dichloro-pyridin-4-yl; 3-chloro-5-fluoro-pyridin-4-yl; 3-fluoro-pyridin-2-yl, 4-fluoro-5-methyl-pyridin-3-yl, 2,6-difluoro-4-methoxyphenyl,2-chlorophenyl, 2-chloro-4-fluorophenyl, 2-chloro-6-fluorophenyl and 2,4-difluorophenyl.

8. The compound according to claim 1, wherein
R⁵ is selected from the group consisting F; Cl; CN; CF₃; CF₂H; CFH₂; C₁₋₄-alkyl; C₃₋₆-cycloalkyl; 3 to 7 membered heterocycloalkyl; O—C₁₋₄-alkyl; OCF₃; OCF₂H; OCFH₂; O—C₃₋₆-cycloalkyl; and —O-(3 to 7 membered heterocycloalkyl),
wherein said C₁₋₄-alkyl is linear or branched, and is unsubstituted or mono- or polysubstituted;
and wherein said C₃₋₆-cycloalkyl and 3 to 7 membered heterocycloalkyl is unsubstituted or mono- or polysubstituted.

9. The compound according to claim 1, wherein
R⁴ and R⁶ are independently selected from the group consisting H; F; Cl; CN; CF₃; CF₂H; CFH₂; C₁₋₄-alkyl; C₃₋₆-cycloalkyl; O—C₁₋₄-alkyl; OCF₃; OCF₂H; and OCFH₂.

10. The compound according to claim 2,
wherein R⁴ and R⁶ are independently selected from the group consisting of H; F and CH₃; or R⁴ is selected from the group consisting of H; F and CH₃ and R⁶ is H.

11. The compound according to claim 1, wherein
L-R³ represents:
C₁₋₄-alkyl; C₃₋₆-cycloalkyl; (3 to 7 membered heterocycloalkyl); O—C₁₋₄-alkyl; OCF₃; OCF₂H; OCFH₂; O—C₃₋₆-cycloalkyl; O-(3 to 7 membered heterocycloalkyl); O—C₁₋₄-alkylene-C₃₋₆-cycloalkyl; O—C₁₋₄-alkylene-(3 to 7 membered heterocycloalkyl); O—C₁₋₄-alkylene-(5- to 6-membered heteroaryl); NH₂; N(H)C₁₋₄-alkyl; N(C₁₋₄-alkyl)₂; NH(C═O)(C₁₋₄-alkyl); N(C₁₋₄-alkyl)(C═O)(C₁₋₄-alkyl); N(H)S(═O)₂(C₁₋₄-alkyl) and N(C₁₋₄-alkyl)S(═O)₂(C₁₋₄-alkyl),
wherein said C₁₋₄-alkyl and C₁₋₄-alkylene is linear or branched, and is unsubstituted or mono- or polysubstituted;
and wherein said 5 to 6-membered heteroaryl, C₃₋₆-cycloalkyl and 3 to 7 membered heterocycloalkyl is unsubstituted or mono- or polysubstituted.

12. The compound according to claim 2, wherein
R¹ denotes CH₃;
R² denotes H;
R⁵ is selected from the group consisting of CF₃; CH₃; OCH₃; OCH₂CH₃; O(CH₂)₂CH₃; OCH(CH₃)₂; O(CH₂)₃CH₃; OCH₂CH(CH₃)₂; OCH(CH₃)CH₂CH₃; OC(CH₃)₃; OCF₃; OCH₂CF₃; OCF₂H; OCFH₂; O-cyclopropyl; O-cyclobutyl; O-cyclopentyl; O-cyclohexyl and O-oxetanyl,
R⁴ and R⁶ denote H;
L-R³ is selected from the group consisting of CH₃; CF₃; cyclopropyl; OCH₃; N(CH₃)₂; OCH₂CH₃; O(CH₂)₂CH₃; OCH(CH₃)₂; OCH₂CH(CH₃)₂; OC(CH₃)₃; OCF₃; OCF₂H; OCFH₂; OCH₂CF₃; O-cyclopropyl; O-cyclobutyl; OCH₂-cyclopropyl; OCH₂-cyclobutyl; O-(3-oxetanyl) and OCH₂-(3-oxetanyl);
and
A is selected from the group consisting of phenyl and pyridinyl,
each unsubstituted or mono- or polysubstituted with substituent(s) independently selected from the group consisting of F; Cl; Br; CN; CF₃; CF₂H; CFH₂; C₁₋₄-alkyl; C₃₋₆-cycloalkyl; OH; O—C₁₋₄-alkyl; OCF₃; OCF₂H; OCFH₂; NH₂; N(H)C₁₋₄-alkyl; N(C₁₋₄-alkyl)₂; and NH(C═O)(C₁₋₄-alkyl).

13. The compound according to claim 1, which is selected from the group consisting of:
1 5-(6-Ethoxy-4-methyl-pyridin-3-yl)-N-(3-fluoro-pyridin-4-yl)-1-methyl-1H-pyrazole-3-carboxylic acid amide;
2 N-(3,5-Difluoro-pyridin-4-yl)-5-(6-ethoxy-4-methyl-pyridin-3-yl)-1-methyl-1H-pyrazole-3-carboxylic acid amide;
3 N-(2,6-Difluoro-phenyl)-5-(6-ethoxy-4-methyl-pyridin-3-yl)-1-methyl-1H-pyrazole-3-carboxylic acid amide;
4 N-(3,5-Difluoro-pyridin-4-yl)-5-(6-ethoxy-2-methyl-pyridin-3-yl)-1-methyl-1H-pyrazole-3-carb oxylic acid amide;
5 N-(2,6-Difluoro-phenyl)-5-(6-ethoxy-2-methyl-pyridin-3-yl)-1-methyl-1H-pyrazole-3-carboxylic acid amide;
6 N-(2,6-Difluoro-phenyl)-5-(6-ethyl-4-methyl-pyridin-3-yl)-1-methyl-1H-pyrazole-3-carb oxylic acid amide;
7 N-(3,5-Difluoro-pyridin-4-yl)-5-(6-ethyl-4-methyl-pyridin-3-yl)-1-methyl-1H-pyrazole-3-carboxylic acid amide;
8 5-(6-Cyclopropyl-4-methyl-pyridin-3-yl)-N-(2,6-difluoro-phenyl)-1-methyl-1H-pyrazole-3-carb oxylic acid amide;
9 5-(6-Cyclopropyloxy-4-methyl-pyridin-3-yl)-N-(3,5-difluoro-pyridin-4-yl)-1-methyl-1H-pyrazole-3-carboxylic acid amide;
10 5-(6-Cyclopropyloxy-4-methyl-pyridin-3-yl)-N-(2,6-difluoro-phenyl)-1-methyl-1H-pyrazole-3-carboxylic acid amide;
11 N-(3,5-Difluoro-pyridin-4-yl)-1-methyl-5-[4-methyl-6-(trifluoromethyloxy)-pyridin-3-yl]-1H-pyrazole-3-carboxylic acid amide;
12 N-(2,6-Difluoro-phenyl)-1-methyl-5-[4-methyl-6-(trifluoromethyloxy)-pyridin-3-yl]-1H-pyrazole-3-carboxylic acid amide;
13 N-(2,6-Difluoro-phenyl)-5-(6-isopropoxy-4-methyl-pyridin-3-yl)-1-methyl-1H-pyrazole-3-carboxylic acid amide;
14 N-(3,5-Difluoro-pyridin-4-yl)-5-(6-ethoxy-4-methoxy-pyridin-3-yl)-1-methyl-1H-pyrazole-3-carboxylic acid amide;
15 5-(4,6-Diethoxy-pyridin-3-yl)-N-(3,5-difluoro-pyridin-4-yl)-1-methyl-1H-pyrazole-3-carboxylic acid amide;
16 N-(2,6-Difluoro-phenyl)-5-(6-ethoxy-4-methoxy-pyridin-3-yl)-1-methyl-1H-pyrazole-3-carboxylic acid amide;
17 5-(4, 6-Diethoxy-pyridin-3-yl)-N-(2,6-difluoro-phenyl)-1-methyl-1H-pyrazole-3-carboxylic acid amide;
18 5-(6-Cyclopropyloxy-4-methyl-pyridin-3-yl)-N-(3,5-difluoro-pyridin-4-yl)-1-methyl-1H-pyrazole-3-carboxylic acid amide;
19-(2,6-Difluoro-phenyl)-1-methyl-5-[4-methyl-6-[(5-methyl-isoxazol-3-yl)-methoxy]-pyridin-3-yl]-1H-pyrazole-3-carboxylic acid amide;
20 5-[6-(Cyclobutyl-methoxy)-4-methyl-pyridin-3-yl]-N-(3,5-difluoro-pyridin-4-yl)-1-methyl-1H-pyrazole-3-carboxylic acid amide;
21 N-(2,6-Difluoro-phenyl)-5-[6-[2-(dimethylamino)ethyloxy]-4-methyl-pyridin-3-yl]-1-methyl-1H-pyrazole-3-carboxylic acid amide;
22 5-(6-Chloro-4-methyl-pyridin-3-yl)-N-(2,6-difluoro-phenyl)-1-methyl-1H-pyrazole-3-carboxylic acid amide;
23 N-(2,6-Difluoro-phenyl)-1-methyl-5-[4-methyl-6-(oxetan-3-yloxy)-pyridin-3-yl]-1H-pyrazole-3-carboxylic acid amide;
24 N-(2,6-Difluoro-phenyl)-5-(6-hydroxy-4-methyl-pyridin-3-yl)-1-methyl-1H-pyrazole-3-carboxylic acid amide;
25 N-(3,5-Difluoro-pyridin-4-yl)-5-(6-hydroxy-4-methyl-pyridin-3-yl)-1-methyl-1H-pyrazole-3-carboxylic acid amide;
26 2-[5-[5-[(2,6-Difluoro-phenyl)-carbamoyl]-2-methyl-2H-pyrazol-3-yl]-4-methyl-pyridin-2-yl]oxy-acetic acid ethyl ester;
27 2-[5-[5-[(2,6-Difluoro-phenyl)-carbamoyl]-2-methyl-2H-pyrazol-3-yl]-4-methyl-pyridin-2-yl]oxy-acetic acid;
28 N-(2,6-Difluoro-phenyl)-5-[6-(2-methoxy-ethoxy)-4-methyl-pyridin-3-yl]-1-methyl-1H-pyrazole-3-carboxylic acid amide;
29 -(2,6-Difluoro-phenyl)-5-[6-[(dimethyl-carbamoylymethoxy]-4-methyl-pyridin-3-yl]-1-methyl-1H-pyrazole-3-carboxylic acid amide;
30 5-[6-(Carbamoyl-methoxy)-4-methyl-pyridin-3-yl]-N-(2,6-difluoro-phenyl)-1-methyl-1H-pyrazole-3-carboxylic acid amide;
31 N-(3,5-Difluoro-pyridin-4-yl)-5-[6-ethoxy-4-(trifluoromethyl)-pyridin-3-yl]-1-methyl-1H-pyrazole-3-carboxylic acid amide;
32 N-(2,6-Difluoro-phenyl)-5-[6-ethoxy-4-(trifluoromethyl)-pyridin-3-yl]-1-methyl-1H-pyrazole-3-carboxylic acid amide;
33 5-(6-Cyclopropyl-5-fluoro-4-methyl-pyridin-3-yl)-N-(2,6-difluoro-phenyl)-1-methyl-1H-pyrazole-3-carboxylic acid amide;
34 5-(6-Cyclopropyl-5-fluoro-4-methyl-pyridin-3-yl)-N-(3,5-difluoro-pyridin-4-yl)-1-methyl-1H-pyrazole-3-carboxylic acid amide;
35 N-(2,6-Difluoro-phenyl)-5-(6-ethoxy-5-fluoro-4-methyl-pyridin-3-yl)-1-methyl-1H-pyrazole-3-carboxylic acid amide;
36 N-(3,5-Difluoro-pyridin-4-yl)-5-(6-ethoxy-5-fluoro-4-methyl-pyridin-3-yl)-1-methyl-1H-pyrazole-3-carboxylic acid amide;
37 N-(2,6-Difluoro-phenyl)-5-(6-dimethylamino-4-methyl-pyridin-3-yl)-1-methyl-1H-pyrazole-3-carboxylic acid amide;
38 N-(2,6-Difluoro-phenyl)-5-(4,6-dimethoxy-pyridin-3-yl)-1-methyl-1H-pyrazole-3-carboxylic acid amide;
39 5-(5-Chloro-2-methyl-pyridin-3-yl)-N-(3-fluoro-pyridin-4-yl)-1-methyl-1H-pyrazole-3-carboxylic acid amide;
40 5-(5-Chloro-2-methyl-pyridin-3-yl)-N-(2,6-difluoro-phenyl)-1-methyl-1H-pyrazole-3-carboxylic acid amide;
41 5-(6-Cyano-4-methyl-pyridin-3-yl)-N-(2,6-difluoro-phenyl)-1-methyl-1H-pyrazole-3-carboxylic acid amide;
42 N-(2,6-Difluoro-phenyl)-5-[4-methoxy-6-(trifluoromethyl)-pyridin-3-yl]-1-methyl-1H-pyrazole-3-carboxylic acid amide;
43 5-[6-(Difluoro-methoxy)-4-methoxy-pyridin-3-yl]-N-(2,6-difluoro-phenyl)-1-methyl-1H-pyrazole-3-carboxylic acid amide;
44 5-[6-(Difluoro-methoxy)-4-methoxy-pyridin-3-yl]-N-(3,5-difluoro-pyridin-4-yl)-1-methyl-1H-pyrazole-3-carboxylic acid amide;
45 5-[6-(Cyclopropyl-methoxy)-4-methoxy-pyridin-3-yl]-N-(2,6-difluoro-phenyl)-1-methyl-1H-pyrazole-3-carboxylic acid amide;
46 N-(2,6-Difluoro-phenyl)-5-[4-methoxy-6-(2,2,2-trifluoro-ethoxy)-pyridin-3-yl]-1-methyl-1H-pyrazole-3-carboxylic acid amide;
47 5-[4-(Difluoro-methoxy)-6-ethoxy-pyridin-3-yl]-N-(2,6-difluoro-phenyl)-1-methyl-1H-pyrazole-3-carboxylic acid amide;
48 5-[6-Cyclopropyl-4-(trifluoromethyl)-pyridin-3-yl]-N-(2,6-difluoro-phenyl)-1-methyl-1H-pyrazole-3-carboxylic acid amide;
49 N-(2,6-Difluoro-phenyl)-5-[6-ethoxy-4-(oxetan-3-yloxy)-pyridin-3-yl]-1-methyl-1H-pyrazole-3-carboxylic acid amide;
50 5-(6-Chloro-4-methoxy-pyridin-3-yl)-N-(2,6-difluoro-phenyl)-1-methyl-1H-pyrazole-3-carboxylic acid amide;
51 N-(2,6-Difluoro-phenyl)-5-[4-methoxy-6-(oxetan-3-yl-methoxy)-pyridin-3-yl]-1-methyl-1H-pyrazole-3-carboxylic acid amide;
52 N-(3,5-Difluoro-pyridin-4-yl)-5-(6-isopropoxy-4-methyl-pyridin-3-yl)-1-methyl-1H-pyrazole-3-carboxylic acid amide;
53 5-(6-Cyclopropyl-4-methoxy-pyridin-3-yl)-N-(3,5-difluoro-pyridin-4-yl)-1-methyl-1H-pyrazole-3-carboxylic acid amide;
54 5-(6-Cyclopropyl-4-methoxy-pyridin-3-yl)-N-(2,6-difluoro-phenyl)-1-methyl-1H-pyrazole-3-carboxylic acid amide;
55 5-[6-(Cyclobutyl-methoxy)-4-methoxy-pyridin-3-yl]-N-(2,6-difluoro-phenyl)-1-methyl-1H-pyrazole-3-carboxylic acid amide;
56 5-(4-Chloro-6-methoxy-pyridin-3-yl)-N-(2,6-difluoro-phenyl)-1-methyl-1H-pyrazole-3-carboxylic acid amide;
57 4-Chloro-N-(2,6-difluoro-phenyl)-5-(4,6-dimethoxy-pyridin-3-yl)-1-methyl-1H-pyrazole-3-carboxylic acid amide;
58 N-(2,6-Difluoro-phenyl)-5-(6-methoxy-4-methyl-pyridin-3-yl)-1-methyl-1H-pyrazole-3-carboxylic acid amide;
59 N-(2,6-Difluoro-phenyl)-5-[6-(methoxymethyl)-4-methyl-pyridin-3-yl]-1-methyl-1H-pyrazole-3-carboxylic acid amide;
60 N-(2,6-Difluoro-phenyl)-1-methyl-5-[4-methyl-6-(trifluoromethyl)-pyridin-3-yl]-1H-pyrazole-3-carboxylic acid amide;
61 5-(6-Cyclopropyloxy-4-methoxy-pyridin-3-yl)-N-(2,6-difluoro-phenyl)-1-methyl-1H-pyrazole-3-carboxylic acid amide;
62 N-(2,6-Difluoro-phenyl)-5-[4-methoxy-6-(oxetan-3-yloxy)-pyridin-3-yl]-1-methyl-1H-pyrazole-3-carboxylic acid amide;
63 5-[6-Cyclopropyl-4-(trifluoromethyl)-pyridin-3-yl]-N-(3,5-difluoro-pyridin-4-yl)-1-methyl-1H-pyrazole-3-carboxylic acid amide;
64 N-(2,6-Difluoro-phenyl)-5-(4,6-dimethoxy-pyridin-3-yl)-1,4-dimethyl-1H-pyrazole-3-carboxylic acid amide;
65 N-(3,5-Difluoro-pyridin-4-yl)-5-(4,6-dimethoxy-pyridin-3-yl)-1,4-dimethyl-1H-pyrazole-3-carboxylic acid amide;
66 5-(4,6-Dimethoxy-pyridin-3-yl)-N-(4,6-dimethyl-pyridin-3-yl)-1,4-dimethyl-1H-pyrazole-3-carboxylic acid amide;
67 5-(4,6-Dimethoxy-pyridin-3-yl)-N-(5-fluoro-4-methyl-pyridin-3-yl)-1,4-dimethyl-1H-pyrazole-3-carboxylic acid amide;
68 N-(2,6-Difluoro-phenyl)-5-(6-ethoxy-4-methoxy-pyridin-3-yl)-1H-pyrazole-3-carboxylic acid amide;
69 N-(2,6-Difluoro-phenyl)-5-(4,6-dimethoxy-pyridin-3-yl)-1H-pyrazole-3-carboxylic acid amide;
70 N-(2,6-Difluoro-phenyl)-5-(6-hydroxy-4-methoxy-pyridin-3-yl)-1-methyl-1H-pyrazole-3-carb oxylic acid amide;
71 N-(2,6-Difluoro-phenyl)-5-(6-ethoxy-4-hydroxy-pyridin-3-yl)-1-methyl-1H-pyrazole-3-carboxylic acid amide
72 5-(3-Cyano-6-trifluoromethyl-pyridin-2-yl)-N-(2,6-difluoro-phenyl)-1-methyl-1H-pyrazole-3-carboxylic acid amide;
73 5-(6-tert-Butyl-3-cyano-pyridin-2-yl)-N-(2,6-difluoro-phenyl)-1-methyl-1H-pyrazole-3-carboxylic acid amide;
74 N-(2,6-Difluoro-phenyl)-5-[2-methoxy-5-(4-methyl-5-oxo-4H-[1,3,4]oxadiazol-2-yl)-pyridin-3-yl]-1-methyl-1H-pyrazole-3-carboxylic acid amide;
75 N-(2,6-Difluoro-phenyl)-5-(4-dimethylamino-6-methoxy-pyridin-3-yl)-1-methyl-1H-pyrazole-3-carboxylic acid amide;
optionally in the form of the free compound and/or a physiologically acceptable salt thereof and/or a physiologically acceptable solvate thereof.

14. A pharmaceutical composition comprising at least one compound according to claim 1 and optionally one or more suitable, pharmaceutically compatible auxiliaries and/or, if appropriate, one or more further pharmacologically active compounds.

15. A method for the treatment and/or prophylaxis in a patient in need thereof of one or more disorders selected from the group consisting of inflammatory disorders, autoimmune diseases, allergic disorders, psoriasis, psoriatic arthritis, rheumatoid arthritis, inflammatory bowel disease, asthma and allergic rhinitis, said method comprising administering to said patient an effective amount therefor of at least one compound according to claim 1.
